(19) United States
(12) Patent Application Publication
(10) Pub. No.: US 2023/0181712 A1
(43) Pub. Date: Jun. 15, 2023

(54) COMBINATION THERAPY WITH MODIFIED PBMCS AND AN IMMUNOCONJUGATE

(71) Applicant: Hoffmann-La Roche Inc., Little Falls, NJ (US)

(72) Inventors: Pablo UMAÑA, Wollerau (CH); Christian KLEIN, Bonstetten (CH); Christine TRUMPFHELLER, Zurich (CH); Valeria G. NICOLINI, Erlenbach (CH); Laura CODARRI DEAK, Au (CH); Scott LOUGHHEAD, Durham, NC (US); Matthew BOOTY, Cambridge, MA (US)

(21) Appl. No.: 17/924,645

(22) PCT Filed: May 10, 2021
(86) PCT No.: PCT/US2021/031542

§ 371 Date: Nov. 10, 2022

Related U.S. Application Data
(60) Provisional application No. 63/023,193, filed on May 11, 2020.
(60) Provisional application No. 63/105,135, filed on Oct. 23, 2020.

(51) Int. Cl. A61K 39/12; A61K 47/68; A61K 35/17; A61K 39/39; C12N 15/117

(52) U.S. Cl. CPC A61K 39/12; A61K 47/6851; A61K 47/6813; A61K 35/17; A61K 39/39; C12N 15/117; A61K 2039/6006

(57) ABSTRACT

The present application provides methods for stimulating an immune response in an individual comprising administering a composition of nucleated cells (e.g., PBMCs) comprising an intracellular exogenous antigen in conjunction with administering an immunoconjugate comprising a variant IL-2 polypeptide and a second polypeptide. The variant IL-2 polypeptide exhibits reduced affinity to the α-subunit of the IL-2 receptor. The second polypeptide targets a tumor cell or a T cell.

### CROSS-REFERENCE TO RELATED APPLICATIONS

This application claims the priority benefit of U.S. Provisional Application No. 63/023,193, filed May 11, 2020, and 63/105,135, filed Oct. 23, 2020, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 202902000140SEQLIST.TXT, date recorded: May 6, 2021, size: 72.6 KB).

### BACKGROUND OF THE INVENTION

Immunotherapy can be divided into two main types of interventions, either passive or active. Passive protocols include administration of pre-activated and/or engineered cells, disease-specific therapeutic antibodies, and/or cytokines. Active immunotherapy strategies are directed at stimulating immune system effector functions in vivo. Several current active protocols include vaccination strategies with disease-associated peptides, lysates, or allogeneic whole cells, infusion of autologous DCs as vehicles for tumor antigen delivery, and infusion of immune checkpoint modulators. See Papaioannou, Nikos E., et al. Annals of translational medicine 4.14 (2016).

CD8+ cytotoxic T lymphocytes (CTL) and CD4+ helper T (Th) cells stimulated by disease-associated antigens have the potential to target and destroy diseased cells; however, current methods for inducing endogenous T cell responses have faced challenges. An approach using peripheral blood mononuclear cells (PBMCs) is described in PCT/US2020/020194.

Interleukin-2 (IL-2), also known as T cell growth factor (TCGF), is a 15.5 kDa globular glycoprotein playing a central role in lymphocyte generation, survival and homeostasis. IL-2 is synthesized mainly by activated T-cells, in particular CD4+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells (reviewed e.g. in Waldmann, Nat Rev Immunol 6, 595-601 (2009); Olejniczak and Kasprzak, Med Sci Monit 14, RA179-89 (2008); Malek, Annu Rev Immunol 26, 453-79 (2008)).

Its ability to expand lymphocyte populations in vivo and to increase the effector functions of these cells confers antitumor effects to IL-2, making IL-2 immunotherapy an attractive treatment option for certain metastatic cancers. Consequently, high-dose IL-2 treatment has been approved for use in patients with metastatic renal-cell carcinoma and malignant melanoma.

On the other hand, IL-2 has a dual function in the immune response in that it not only mediates expansion and activity of effector cells, but also is crucially involved in maintaining peripheral immune tolerance. Therefore, IL-2 is not optimal for inhibiting tumor growth, because in the presence of IL-2 either the CTLs generated might recognize the tumor as self and undergo AICD or the immune response might be inhibited by IL-2 dependent T_{reg} cells. A further concern in relation to IL-2 immunotherapy are the side effects produced by recombinant human IL-2 treatment. Patients receiving high-dose IL-2 treatment frequently experience severe cardiovascular, pulmonary, renal, hepatic, gastrointestinal, neurological, cutaneous, hematological and systemic adverse events, which require intensive monitoring and in-patient management. The majority of these side effects can be explained by the development of so-called vascular (or capillary) leak syndrome (VLS), a pathological increase in vascular permeability leading to fluid extravasation in multiple organs (causing e.g. pulmonary and cutaneous edema and liver cell damage) and intravascular fluid depletion (causing a drop in blood pressure and compensatory increase in heart rate).

A particular mutant IL-2 polypeptide, designed to overcome the above-mentioned problems associated with IL-2 immunotherapy (toxicity caused by the induction of VLS, tumor tolerance caused by the induction of AICD, and immunosuppression caused by activation of Treg cells), is described in WO 2012/107417. Substitution of the phenylalanine residue at position 42 by alanine, the tyrosine residue at position 45 by alanine and the leucine residue at position 72 of IL-2 by glycine essentially abolishes binding of this mutant IL-2 polypeptide to the α-subunit of the IL-2 receptor (CD25).

Further to the above-mentioned approaches, IL-2 immunotherapy may be improved by selectively targeting IL-2 to tumors, e.g. in the form of immunoconjugates comprising an antibody that binds to an antigen expressed on tumor cells. Several such immunoconjugates have been described (see e.g. Ko et al., J Immunother (2004) 27, 232-239; Klein et al., Oncoimmunology (2017) 6(3), e1277306).

Tumors may be able, however, to escape such targeting by shedding, mutating or downregulating the target antigen of the antibody. Moreover, tumor-targeted IL-2 may not come into optimal contact with effector cells such as cytotoxic T lymphocytes (CTLs), in tumor microenvironments that actively exclude lymphocytes. Thus, there remains a need to further improve IL-2 immunotherapy. An approach, which may circumvent the problems of tumor-targeting, is to target IL-2 directly to effector cells, in particular CTLs. A fusion protein of IL-2 and a PD-1 antigen-binding protein is described in WO 2018/184964 A1.

There remains a need for improved methods for inducing an effective endogenous T cell response.

All references cited herein, including patent applications and publications, are incorporated by reference in their entirety.

### BRIEF SUMMARY OF THE INVENTION

In some aspects, the invention provides a method for stimulating an immune response in an individual, the method comprising a) administering an effective amount of a composition comprising nucleated cells to an individual, wherein the nucleated cells comprise an exogenous antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

In some aspects, the invention provides a method for stimulating an immune response to a tumor antigen in an individual, the method comprising a) administering an effective amount of a composition comprising nucleated cells to an individual, wherein the nucleated cells comprise an exogenous tumor antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

In some aspects, the invention provides a method for enhancing a nucleated cell-based immunotherapy, the method comprising administering an effective amount of an immunoconjugate in conjunction with the nucleated cell-based immunotherapy, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment.

In some aspects, the invention provides a method for treating a disease in an individual, the method comprising a) administering an effective amount of a composition comprising nucleated cells to an individual, wherein the nucleated cells comprise an exogenous antigen associated with the disease; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment. In some embodiments, the disease is cancer, an infectious disease, or a viral-associated disease.

In some aspects, the invention provides a method of vaccinating an individual in need thereof, the method comprising a) administering an effective amount of a composition comprising nucleated cells to an individual, wherein the nucleated cells comprise an exogenous antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment. In some embodiments, the individual has a disease responsive to vaccination. In some embodiments, the disease is cancer, an infectious disease, or a viral-associated disease.

In some aspects, the invention provides a method for reducing tumor growth in an individual, the method comprising a) administering an effective amount of a composition comprising nucleated cells to an individual, wherein the nucleated cells comprise an exogenous tumor antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

In some embodiments of the invention, the second polypeptide binds a T cell. In some embodiments, the second polypeptide binds PD-1 expressed on the T cell. In some embodiments, the second polypeptide is an antigen-binding moiety that specifically binds PD-1. In some embodiments, the anti-PD-1 antigen-binding moiety comprises (a) a heavy chain variable region (VH) comprising a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6; or (b) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO: 13. In some embodiments, the anti-PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO:14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO:17, and SEQ ID NO:18. In some embodiments, the anti-PD-1 antigen-binding moiety comprises (a) a heavy chain variable region (VH) comprising an amino acid sequence of SEQ ID NO:14, and (b) a light chain variable region (VL) comprising an amino acid sequence of SEQ ID NO:15, SEQ ID NO: 16, SEQ ID NO: 17, and SEQ ID NO:18. In some embodiments, the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO:15. In some embodiments, the immunoconjugate comprises a polypeptide comprising an amino acid sequence that is at least about 80%, 85%, 90%, 95%, 96%, 97%, 98%, 99% or 100% identical to the sequence of SEQ ID NO:22, a polypeptide comprising an amino acid sequence that is at least about 80%, 85%, 90%, 95%, 96%, 97%, 98%, 99% or 100% identical to the sequence of SEQ ID NO:24, and a polypeptide comprising an amino acid sequence that is at least about 80%, 85%, 90%, 95%, 96%, 97%, 98%, 99% or 100% identical to the sequence of SEQ ID NO:25. In some embodiments, the immunoconjugate comprises a polypeptide sequence of SEQ ID NO:22, a polypeptide sequence of SEQ ID NO:24, and two polypeptide sequences of SEQ ID NO:25.

In some embodiments of the invention, the second polypeptide specifically binds a target antigen presented on a tumor cell or in a tumor cell environment. In some embodiments, the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP). In some embodiments, the second polypeptide binds FAP. In some embodiments, the second polypeptide is an antigen-binding moiety that specifically binds FAP. In some embodiments, the antigen-binding moiety the specifically bind FAP comprising (i) the heavy chain variable region sequence of SEQ ID NO:29 and the light chain variable region sequence of SEQ ID NO: 28; (ii) the heavy chain variable region sequence of SEQ ID NO:31 and the light chain variable region sequence of SEQ ID NO:30; (iii) the heavy chain variable region sequence of SEQ ID NO: 33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36. In some embodiments, the antigen-binding moiety the specifically bind FAP comprising (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO: 231; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41. In some embodiments, antigen-binding moiety the specifically bind FAP comprises the heavy chain variable region sequence of SEQ ID NO:33 and the light chain variable region sequence of SEQ ID NO:32. In some embodiments, the immunoconjugate comprises a polypeptide sequence of SEQ ID NO:38, a polypeptide sequence of SEQ ID NO:39, and a polypeptide sequence of SEQ ID NO:40.

In some embodiments of the invention, the mutant IL-2 polypeptide further comprises the amino acid substitution T3A and/or the amino acid substitution C125A. In some embodiments, the mutant IL-2 polypeptide comprises an amino acid sequence that is at least about 95%, 96%, 97%, 98%, 99% or 100% identical to the amino acid sequence of SEQ ID NO:20, wherein the mutant IL-2 polypeptide shows reduced affinity to the high-affinity IL-2 receptor and substantially similar affinity to the intermediate-affinity IL-2 receptor, each compared to a wild-type IL-2 polypeptide. In some embodiments, the mutant IL-2 polypeptide comprises the amino acid sequence of SEQ ID NO:20.

In some embodiments of the invention, the nucleated cells are immune cells. In some embodiments, the nucleated cells are a plurality of peripheral blood mononuclear cells (PBMCs). In some embodiments, the plurality of PBMCs comprise two or more of T cell, B cell, NK cell, monocytes, dendritic cells or NK-T cells. In some embodiments, the nucleated cells are one or more of T cells, B cells, NK cells, monocytes, dendritic cells and/or NK-T cells.

In some embodiments of the invention, the exogenous antigen is delivered to the nucleated cells intracellularly. In some embodiments, the exogenous antigen is a disease-associated antigen. In some embodiments, the disease-associated antigen is a cancer antigen, an infectious disease antigen, or a viral-associated disease antigen. In some embodiments, the exogenous antigen is a human papillomavirus (HPV) antigen.

In some embodiments of the invention, the composition further comprises an adjuvant. In some embodiments, the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

In some embodiments of the invention, the nucleated cells comprising the exogenous antigen are prepared by a process comprising the steps of: a) passing a cell suspension comprising input nucleated cells through a cell-deforming constriction, wherein a diameter of the constriction is a function of a diameter of the input nucleated cells in the suspension, thereby causing perturbations of the input nucleated cells large enough for the exogenous antigen to pass through to form perturbed input nucleated cells; b) incubating the perturbed input nucleated cells with the exogenous antigen for a sufficient time to allow the exogenous antigen to enter the perturbed input nucleated cells, thereby generating nucleated cells comprising the exogenous antigen. In some embodiments, the width of the constriction is about 10% to about 99% of the mean diameter of the input nucleated cells. In some embodiments, the width of the constriction is about 4.2 μm to about 6 μm. In some embodiments, the width of the constriction is about 4.2 μm to about 4.8 μm. In some embodiments, the width of the constriction is about 4.5 μm. In some embodiments, the cell suspension comprising the plurality of input nucleated cells are passed through multiple constrictions wherein the multiple constrictions are arranged in series and/or in parallel. In some embodiments, the exogenous antigen is present in at least about 70% of the nucleated cells following incubation of the perturbed input nucleated cells with the exogenous antigen.

In some embodiments of the invention, the nucleated cells are conditioned with an adjuvant to form conditioned cells. In some embodiments, the nucleated cells are incubated with the adjuvant for about 1 hour to about 24 hours, about 2 hours to about 10 hours, about 3 hours to about 6 hours, or about 4 hours for the cells to condition. In some embodiments, the nucleated cells are conditioned before or after introducing the exogenous antigen into the nucleated cells. In some embodiments, the adjuvant is a CpG oligodeoxynucleotide (ODN), LPS, IFN-α, STING agonists, RIG-I agonists, poly I.C, R837, R848, a TLR3 agonist, a TLR4 agonist or a TLR 9 agonist. In some embodiments, the adjuvant is a CpG oligodeoxynucleotide (ODN). In some embodiments, the adjuvant is CpG 7909. In some embodiments, the conditioned cells are a conditioned plurality of modified PBMCs. In some embodiments, the plurality of modified PBMCs are further modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112. In some embodiments, the plurality of modified PBMCs are further modified to increase expression of one or more cytokines. In some embodiments, the cytokine is IL-15, IL-12, IL-2, IFN-α, or IL-21. In some embodiments, one or more co-stimulatory molecules is upregulated in the B cells of the conditioned plurality of modified PBMCs compared to the B cells in the plurality of nonmodified PBMCs, wherein the co-stimulatory molecule is CD80 and/or CD86. In some embodiments, the plurality of modified PBMCs have increased expression of one or more of IFN-γ, IL-6, MCP-1, MIP-1β, IP-10, or TNF-α compared to a plurality of unconditioned PBMCs. In some embodiments, the expression of one or more of IFN-γ, IL-6, MCP-1, MIP-1β, IP-10, or TNF-α is increased by more than about 1.2-fold, 1.5-fold, 1.8-fold, 2-fold, 3-fold, 4-fold, 5-fold, 8-fold, or more than 10-fold compared to the plurality of unconditioned PBMCs.

In some embodiments of the invention, the immunoconjugate is administered before, at the same time, or after administration of the composition comprising nucleated cells. In some embodiments, the composition comprising nucleated cells is administered a plurality of times. In some embodiments, the immunoconjugate is administered a plurality of times after administration of the composition comprising nucleated cells. In some embodiments, the composition and/or the immunoconjugate is administered intravenously. In some embodiments, the immunoconjugate is administered subcutaneously or intratumorally. In some embodiments, the immunoconjugate is administered subcutaneously in combination with hyaluronidase.

In some embodiments of the invention, the individual is a human. In some embodiments, the individual has cancer, an infectious disease, or a viral associated disease. In some embodiments, the composition of nucleated cells and/or the immunoconjugate is administered prior to, concurrently with, or following administration of another therapy. In some embodiments, the another therapy is a chemotherapy or a radiation therapy.

In some aspects, the invention provides a composition comprising nucleated cells comprising an exogenous antigen for use in a method for treating a disease in an individual, wherein the composition is used in combination with an immunoconjugate comprising a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19). In some embodiments, the disease is cancer, an infectious disease, or a viral-associated disease. In some embodiments, the composition comprising nucleated cells is administered before, at the same time, or after the immunoconjugate.

In some aspects, the invention provides an immunoconjugate comprising a mutant IL-2 polypeptide and a second polypeptide capable of specific binding to a T cell, a tumor cell, or the tumor cell environment for use in a method for treating a disease in an individual, wherein the immunoconjugate is used in combination with a composition comprising nucleated cells comprising an exogenous antigen. In some embodiments, the disease is cancer, an infectious disease, or a viral-associated disease. In some embodiments, the composition comprising the nucleated cells is administered before, at the same time, or after the immunoconjugate.

In some aspects, the invention provides the use of an effective amount of an immunoconjugate in the manufacture of a medicament for stimulating an immune response in an individual, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19); wherein the immunoconjugate is formulated for administration in conjunction with a composition comprising nucleated cells, wherein the nucleated cells comprise an exogenous antigen. In some embodiments, the immunoconjugate is administered before, at the same time, or after the composition comprising nucleated cells.

In some aspects, the invention provides the use of an effective amount of a composition in the manufacture of a medicament for stimulating an immune response in an individual, wherein the composition comprises nucleated cells comprising an exogenous antigen; wherein the composition is formulated for administration in conjunction with an immunoconjugate comprising a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19). In some embodiments, the composition comprising the nucleated cells is administered before, at the same time, or after the immunoconjugate.

In some aspects, the invention provides a kit for use in any of the methods described herein. In some embodiments, the invention provides a kit comprising a composition of nucleated cells comprising an exogenous antigen and an immunoconjugate, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19); wherein the composition and the immunoconjugate are for use in combination for stimulating an immune response to the exogenous antigen in an individual. In some embodiments, the invention provides a kit comprising a composition of nucleated cells comprising an exogenous antigen, wherein the composition is for use in conjunction with an immunoconjugate for stimulating an immune response to the exogenous antigen in an individual; wherein the immunoconjugate comprises a mutant IL-2 polypeptide and a second polypeptide capable of specific binding to a T cell, a tumor cell, or the tumor cell environment; and wherein the mutant IL-2 polypeptide is a human IL-2 molecule comprising the amino acid substitutions F42A, Y45A and L72G (numbering relative to the human IL-2 sequence SEQ ID NO: 19). In some embodiments, the invention provides a kit comprising an immunoconjugate, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19); wherein the immunoconjugate is for use in conjunction with a composition of nucleated cells comprising an exogenous antigen for stimulating an immune response to the exogenous antigen in an individual.

In some aspects, the invention provides a method for producing an immunoconjugate for use in conjunction with a composition comprising nucleated cells for stimulating an immune response in an individual, the method comprising expressing a nucleic acid encoding the immunoconjugate in a cell under conditions to produce the immunoconjugate, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19); wherein the immunoconjugate is for use in conjunction with administering a composition comprising nucleated cells, wherein the nucleated cells comprise an exogenous antigen. In some embodiments, the immunoconjugate is a fusion protein.

In some aspects, the invention provides a method for producing a composition comprising nucleated cells for use in conjunction with an immunoconjugate for stimulating an immune response in an individual, the method comprising introducing an exogenous antigen intracellularly to a population of nucleated cells; wherein the composition is for use in conjunction with administration of an immunoconjugate; wherein the immunoconjugate comprises a mutant IL-2 polypeptide and a second polypeptide capable of specific binding to a T cell, a tumor cell, or the tumor cell environment; and wherein the mutant IL-2 polypeptide is a human IL-2 molecule comprising the amino acid substitutions F42A, Y45A and L72G (numbering relative to the human IL-2 sequence SEQ ID NO: 19).

### BRIEF DESCRIPTION OF THE DRAWINGS

FIG. 1 is a graph showing tumor volume over time in a TC1 mouse tumor model. Mice were treated with PD1-IL2v alone, with SQZ-PBMC (SQZ-PBMC comprising an HPV antigen delivered intracellularly) once with at three doses of cells or PD1-IL2v was given three times with a single dose range. Administration of SQZ-PBMC is indicated as “SQZ Prime.” Period of administration of PD1-IL2v is indicated by the gray bar.

FIGS. 2A-2F are graphs showing tumor volume in the TC1 mouse model treated with SQZ-PBMC alone compared to untreated (FIGS. 2A-2C) or in combination with a fixed dose of PD1-IL2v compared to untreated (FIGS. 2D-2F). Doses of SQZ-PBMC were 0.25×10⁶ (FIGS. 2A and 2D), 1.0×10⁶ (FIGS. 2B and 2E), or 4.0×10⁶ (FIGS. 2C and 2F). Administration of SQZ-PBMC is indicated by the dashed vertical line. Period of administration of PD1-IL2v is indicated by the gray bar.

FIGS. 3A and 3B are graphs showing tumor volume in a TC1 mouse tumor model in mice treated with SQZ-PBMC in combination with FAP-IL-2v (FIG. 3A) and PD-1-IL-2v (FIG. 3B). SQZ-PBMC and immunoconjugate administration is indicated by the dashed vertical lines.

FIGS. 4A-4E are spider plot graphs showing tumor volume in the TC1 mouse model treated with SQZ-PBMC alone compared to untreated (FIG. 4A), FAP-IL2v alone compared to untreated (FIG. 4B), PD-1-IL-2v alone compared to untreated (FIG. 4C) FAP-IL2v in combination with SQZ-PBMC compared to untreated (FIG. 4D), or PD-1-IL-2v in combination with SQZ-PBMC compared to untreated (FIG. 4E). Administration of SQZ-PBMC and immunoconjugates is indicated by the dashed vertical lines.

FIG. 5 is a graph showing tumor volume in a TC1 mouse tumor model in mice treated with PD-1-IL-2v, SQZ-PBMC, or a combination of PD-1-IL-2v and SQZ-PBMC. SQZ-PBMC and immunoconjugate administration is indicated by the dashed vertical lines.

FIG. 6 is a graph showing re-challenge following treatment of an original tumor. TC1 tumors established in the right flank of mice were treated with 0.25×10⁶, 1×10⁶ or 4×10⁶ SQZ-PBMC and with PD1-IL2v alone. Mice were rechallenged with a TC1 tumor in the left flank.

FIGS. 7A-7D are graphs showing the analysis of Tumor-infiltrating leukocytes (TIL) in tumors following treatment with SQZ-PBMC and PD-1-IL2v. FIG. 7A shows proliferation of T cells in tumors as measured by % Ki-67+ cells. FIG. 7B shows cytotoxicity as measured by Granzyme B mean fluorescent intensity (MFI). FIG. 7C shows IFNγ and FIG. 7D shows TNFα production in E7-stimulated samples and unstimulated samples. Untreated indicated as Untr, PD-1-IL2v alone indicates as PD-1-IL2v, SQZ-PBMC alone indicated as SQZ, and the combination of SQZ-PBMC and PD-1-IL2v is indicated as Combo. For all graphs, statistical significance is indicated by ** is p<0.01, *** is p<0.001.

FIGS. 8A-8H are a series of graphs showing immune infiltration of tumors at days 24 (FIGS. 8A-8D) and 28 days (FIGS. 8E-8H) following treatment in a TC1 mouse tumor model. Parameters presented include tumor volume (FIGS. 8A and 8E), number of CD45+ cells (FIGS. 8B and 8F), number of CD8+ T cells (FIGS. 8C and 8G), and E7-specific CD8+ cells (FIGS. 8D and 8H). Untreated indicated as Untr, PD-1-IL2v alone indicates as PD-1-IL2v, SQZ-PBMC alone indicated as SQZ, and the combination of SQZ-PBMC and PD-1-IL2v is indicated as Combo. For all graphs, statistical significance is indicated by * is p<0.05, ** is p<0.01, *** is p<0.001.

FIGS. 9A-9D show the number of immune cells within tumors and spleens of mice following treatment. FIG. 9A shows the number of CD8+ T cells in tumors. FIG. 9B shows the number of E7-specific CD8+ T cells in tumors. FIG. 9C shows the number of CD8+ T cells in spleen. FIG. 9D shows the number of E7-specific CD8+ T cells in spleen. Untreated indicated as Untr, PD-1-IL2v alone indicates as PD-1-IL2v, SQZ-PBMC alone indicated as SQZ, and the combination of SQZ-PBMC and PD-1-IL2v is indicated as Combo. For all graphs, statistical significance is indicated by * is p<0.05, ** is p<0.01, *** is p<0.001. The fold-difference between selected values is also indicated.

FIGS. 10A-10D show the effects of treatment on regulatory T cells (Tregs) and NK cells in tumors. FIG. 10A shows the number of Tregs as measured by the number of CD4+ FOXP3+CD25+ cells per mg of tumor. FIG. 10B shows the ratios of CD8+ cells to Treg cells. FIG. 10C shows the numbers of NK1.1+ cells in tumors and FIG. 10D shows the number of NK1.1+ cells in spleens. Untreated indicated as Untr, PD-1-IL2v alone indicates as PD-1-IL2v, SQZ-PBMC alone indicated as SQZ, and the combination of SQZ-PBMC and PD-1-IL2v is indicated as Combo. For all graphs, statistical significance is indicated by ** is p<0.01, *** is p<0.001, and **** p<0.0001.

FIGS. 11A-11C show the effects of a combination of a peptide vaccine and immunoconjugate treatment in a TC1 mouse tumor model. Mice were treated with the peptide vaccine alone (VAX, FIG. 11A), the vaccine with FAP-IL2v (FIG. 11B), or the vaccine with PD1-IL2v (FIG. 11C).

FIG. 12 showing that administration of PD1-IL2v after SQZ-PBMC immunization boosts antigen-specific CD8+ T cell responses.

### DETAILED DESCRIPTION OF THE INVENTION

In some aspects, the invention provides methods for stimulating an immune response in an individual, the method comprising a) administering an effective amount of a composition comprising nucleated cells to an individual, wherein the nucleated cells comprise an exogenous antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment. In some aspects, the invention provides methods for stimulating an immune response to a tumor antigen in an individual, the method comprising a) administering an effective amount of a composition comprising nucleated cells to an individual, wherein the nucleated cells comprise an exogenous tumor antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment. In some aspects, the invention provides methods for enhancing a nucleated cell-based immunotherapy, the method comprising administering an effective amount of an immunoconjugate in conjunction with the nucleated cell-based immunotherapy, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment. In some aspects, the invention provides methods for treating a disease in an individual, the method comprising a) administering an effective amount of a composition comprising nucleated cells to an individual, wherein the nucleated cells comprise an exogenous antigen associated with the disease; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment. In some aspects, the invention provides methods of vaccinating an individual in need thereof, the method comprising a) administering an effective amount of a composition comprising nucleated cells to an individual, wherein the nucleated cells comprise an exogenous antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment. In some aspects, the invention provides methods for reducing tumor growth in an individual, the method comprising a) administering an effective amount of a composition comprising nucleated cells to an individual, wherein the nucleated cells comprise an exogenous tumor antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment. In some embodiments or the above aspects, the second polypeptide is capable of specific binding to PD-1. In some embodiments of the above aspects, the second polypeptide is capable of specific binding to FAP.

In some embodiments, the invention provides compositions of nucleated cells comprising an exogenous antigen for use in combination with an immunoconjugate comprising a mutant IL-2 polypeptide and a second polypeptide capable of specific binding to a T cell, a tumor cell, or the tumor cell environment. In some embodiments, the invention provides an immunoconjugate comprising a mutant IL-2 polypeptide and a second polypeptide capable of specific binding to a T cell, a tumor cell, or the tumor cell environment, for use in combination with compositions of nucleated cells comprising an exogenous antigen. Uses and kits of the compositions and immunoconjugate described above are also contemplated.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al., 4th ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6th ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011).

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 19. Unprocessed human IL-2 additionally comprises an N-terminal 20 amino acid signal peptide having the sequence of SEQ ID NO:59, which is absent in the mature IL-2 molecule.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO:19). The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as a mutant IL-2 peptide sequence, a mutant IL-2 polypeptide, a mutant IL-2 protein or a mutant IL-2 analog.

Designation of various forms of IL-2 is herein made with respect to the sequence shown in SEQ ID NO: 19. Various designations may be used herein to indicate the same mutation. For example a mutation from phenylalanine at position 42 to alanine can be indicated as 42A, A42, A42, F42A, or Phe42Ala.

By a “human IL-2 molecule” as used herein is meant an IL-2 molecule comprising an amino acid sequence that is at least about 90%, at least about 91%, at least about 92%, at least about 93%, at least about 94%, at least about 95% or at least about 96% identical to the human IL-2 sequence of SEQ ID NO: 19. Particularly, the sequence identity is at least about 95%, more particularly at least about 96%. In particular embodiments, the human IL-2 molecule is a full-length IL-2 molecule.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g. reduced binding to CD25. Amino acid sequence deletions and insertions include amino- and/or carboxy-terminal deletions and insertions of amino acids. An example of a terminal deletion is the deletion of the alanine residue in position 1 of full-length human IL-2. Preferred amino acid mutations are amino acid substitutions. For the purpose of altering e.g. the binding characteristics of an IL-2 polypeptide, non-conservative amino acid substitutions, i.e. replacing one amino acid with another amino acid having different structural and/or chemical properties, are particularly preferred. Preferred amino acid substitutions include replacing a hydrophobic by a hydrophilic amino acid. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

As used herein, a “wild-type” form of IL-2 is a form of IL-2 that is otherwise the same as the mutant IL-2 polypeptide except that the wild-type form has a wild-type amino acid at each amino acid position of the mutant IL-2 polypeptide. For example, if the IL-2 mutant is the full-length IL-2 (i.e. IL-2 not fused or conjugated to any other molecule), the wild-type form of this mutant is full-length native IL-2. If the IL-2 mutant is a fusion between IL-2 and another polypeptide encoded downstream of IL-2 (e.g. an antibody chain) the wild-type form of this IL-2 mutant is IL-2 with a wild-type amino acid sequence, fused to the same downstream polypeptide. Furthermore, if the IL-2 mutant is a truncated form of IL-2 (the mutated or modified sequence within the non-truncated portion of IL-2) then the wild-type form of this IL-2 mutant is a similarly truncated IL-2 that has a wild-type sequence. For the purpose of comparing IL-2 receptor binding affinity or biological activity of various forms of IL-2 mutants to the corresponding wild-type form of IL-2, the term wild-type encompasses forms of IL-2 comprising one or more amino acid mutation that does not affect IL-2 receptor binding compared to the naturally occurring, native IL-2, such as e.g. a substitution of cysteine at a position corresponding to residue 125 of human IL-2 to alanine. In some embodiments wild-type IL-2 for the purpose of the present invention comprises the amino acid substitution C125A (see SEQ ID NO:26). In certain embodiments according to the invention the wild-type IL-2 polypeptide to which the mutant IL-2 polypeptide is compared comprises the amino acid sequence of SEQ ID NO: 19. In other embodiments, the wild-type IL-2 polypeptide to which the mutant IL-2 polypeptide is compared comprises the amino acid sequence of SEQ ID NO:26.

The term “CD25” or “α-subunit of the IL-2 receptor” as used herein, refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments, CD25 is human CD25. The amino acid sequence of human CD25 is found e.g. in UniProt entry no. P01589 (version 185).

The term “high-affinity IL-2 receptor” as used herein refers to the heterotrimeric form of the IL-2 receptor, consisting of the receptor γ-subunit (also known as common cytokine receptor γ-subunit, γc, or CD132, see UniProt entry no. P14784 (version 192)), the receptor β-subunit (also known as CD122 or p70, see UniProt entry no. P31785 (version 197)) and the receptor α-subunit (also known as CD25 or p55, see UniProt entry no. P01589 (version 185)). The term “intermediate-affinity IL-2 receptor” by contrast refers to the IL-2 receptor including only the γ-subunit and the β-subunit, without the α-subunit (for a review see e.g. Olejniczak and Kasprzak, Med Sci Monit 14, RA179-189 (2008)).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen-binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

The affinity of the mutant or wild-type IL-2 polypeptide for various forms of the IL-2 receptor can be determined in accordance with the method set forth in the WO 2012/107417 by surface plasmon resonance (SPR), using standard instrumentation such as a BIAcore instrument (GE Healthcare) and receptor subunits such as may be obtained by recombinant expression (see e.g. Shanafelt et al., Nature Biotechnol 18, 1197-1202 (2000)). Alternatively, binding affinity of IL-2 mutants for different forms of the IL-2 receptor may be evaluated using cell lines known to express one or the other such form of the receptor. Specific illustrative and exemplary embodiments for measuring binding affinity are described hereinafter.

By “regulatory T cell” or “Treg cell” is meant a specialized type of CD4+ T cell that can suppress the responses of other T cells. Treg cells are characterized by expression of the α-subunit of the IL-2 receptor (CD25) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. Treg cells require IL-2 for their function and development and induction of their suppressive characteristics.

As used herein, the term “effector cells” refers to a population of lymphocytes that mediate the cytotoxic effects of IL-2. Effector cells include effector T cells such as CD8+ cytotoxic T cells, NK cells, lymphokine-activated killer (LAK) cells and macrophages/monocytes.

As used herein, the term “PD1”, “human PD1”, “PD-1” or “human PD-1” (also known as Programmed cell death protein 1, or Programmed Death 1) refers to the human protein PD1. See also UniProt entry no. Q15116 (version 156). As used herein, an antibody “binding to PD-1”, “specifically binding to PD-1”, “that binds to PD-1” or “anti-PD-1 antibody” refers to an antibody that is capable of binding PD-1, especially a PD-1 polypeptide expressed on a cell surface, with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, e.g., by radioimmunoassay (RIA) or flow cytometry (FACS) or by a Surface Plasmon Resonance assay using a biosensor system such as a Biacore® system. In certain embodiments, an antibody that binds to PD-1 has a KD value of the binding affinity for binding to human PD-1 of ≤1 μM, ≤100 nM, ≤10 nM, 1 nM, ≤0.1 nM, ≤0.01 nM, or ≤0.001 nM (e.g. 10-8 M or less, e.g. from 10⁻⁸M to 10⁻¹³ M, e.g., from 10⁻⁹ M to 10⁻¹³ M). In one embodiment, the K_{D} value of the binding affinity is determined in a Surface Plasmon Resonance assay using the Extracellular domain (ECD) of human PD-1 (PD-1-ECD) as antigen.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. In one embodiment, the antigen-binding molecule of the invention is capable of specific binding to human, mouse and/or cynomolgus FAP. The amino acid sequence of human FAP is shown in UniProt (world wide web.uniprot.org) accession no. Q12884 (version 149), or NCBI (world wide web.ncbi.nlm.nih.gov/) RefSeq NP_004451.2. The extracellular domain (ECD) of human FAP extends from amino acid position 26 to 760. The amino acid sequence of mouse FAP is shown in UniProt accession no. P97321 or NCBI RefSeq NP_032012.1. The extracellular domain (ECD) of mouse FAP extends from amino acid position 26 to 761. In some embodiments, an anti-FAP binding molecule of the invention binds to the extracellular domain of FAP. Exemplary anti-FAP binding molecules are described in International Patent Application No. WO 2012/020006 A2. In one embodiment, the extent of binding of an anti-FAP antibody to an unrelated, non-FAP protein is less than about 10% of the binding of the antibody to FAP as measured, e.g., by radioimmunoassay (RIA) or flow cytometry (FACS) or by a Surface Plasmon Resonance assay using a biosensor system such as a Biacore® system. In certain embodiments, an antibody that binds to FAP has a K_{D} value of the binding affinity for binding to human FAP of ≤1 μM, ≤100 nM, ≤10 nM, 1 nM, ≤0.1 nM, ≤0.01 nM, or ≤0.001 nM (e.g., 10⁻⁸ M or less, e.g. from 10⁻⁸ M to 10⁻¹³ M, e.g., from 10⁻⁹ M to 10⁻¹³ M). In one embodiment, the K_{D} value of the binding affinity is determined in a Surface Plasmon Resonance assay using the Extracellular domain (ECD) of human FAP as antigen.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The term “monoclonal antibody” as used herein refers to an antibody obtained from a population of substantially homogeneous antibodies, i.e. the individual antibodies comprised in the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment, i.e. that is not in its natural milieu. No particular level of purification is required. For example, an isolated antibody can be removed from its native or natural environment. Recombinantly produced antibodies expressed in host cells are considered isolated for the purpose of the invention, as are native or recombinant antibodies which have been separated, fractionated, or partially or substantially purified by any suitable technique. As such, the immunoconjugates of the present invention are isolated. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007).

The terms “full-length antibody”, “intact antibody”, and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and single-domain antibodies. For a review of certain antibody fragments, see Holliger and Hudson, Nature Biotechnology 23:1126-1136 (2005).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called a (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen-binding domain” refers to the part of an antibody that comprises the area which specifically binds to and is complementary to part or all of an antigen. An antigen-binding domain may be provided by, for example, one or more antibody variable domains (also called antibody variable regions). Particularly, an antigen-binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, vMD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, VMD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs; three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. Such variable domains are referred to herein as “humanized variable region”. A humanized antibody optionally may comprise at least a portion of an antibody constant region derived from a human antibody. In some embodiments, some FR residues in a humanized antibody are substituted with corresponding residues from a non-human antibody (e.g., the antibody from which the HVR residues are derived), e.g., to restore or improve antibody specificity or affinity. A “humanized form” of an antibody, e.g. of a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In certain embodiments, a human antibody is derived from a non-human transgenic mammal, for example a mouse, a rat, or a rabbit. In certain embodiments, a human antibody is derived from a hybridoma cell line. Antibodies or antibody fragments isolated from human antibody libraries are also considered human antibodies or human antibody fragments herein.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprised in an immunoconjugate according to the invention, comprises an additional C-terminal glycine-lysine dipeptide (G446 and K447, numbering according to EU index of Kabat). In one embodiment of the invention, a heavy chain including a subunit of an Fc domain as specified herein, comprised in an immunoconjugate according to the invention, comprises an additional C-terminal glycine residue (G446, numbering according to EU index of Kabat). Compositions of the invention, such as the pharmaceutical compositions described herein, comprise a population of immunoconjugates of the invention. The population of immunoconjugates may comprise molecules having a full-length heavy chain and molecules having a cleaved variant heavy chain. The population of immunoconjugates may consist of a mixture of molecules having a full-length heavy chain and molecules having a cleaved variant heavy chain, wherein at least 50%, at least 60%, at least 70%, at least 80% or at least 90% of the immunoconjugates have a cleaved variant heavy chain. In one embodiment of the invention, a composition comprising a population of immunoconjugates of the invention comprises an immunoconjugate comprising a heavy chain including a subunit of an Fc domain as specified herein with an additional C-terminal glycine-lysine dipeptide (G446 and K447, numbering according to EU index of Kabat). In one embodiment of the invention, a composition comprising a population of immunoconjugates of the invention comprises an immunoconjugate comprising a heavy chain including a subunit of an Fc domain as specified herein with an additional C-terminal glycine residue (G446, numbering according to EU index of Kabat). In one embodiment of the invention, such a composition comprises a population of immunoconjugates comprised of molecules comprising a heavy chain including a subunit of an Fc domain as specified herein; molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen-binding moieties) are not the same. In some embodiments, the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” when used in reference to antibodies refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

“Reduced binding”, for example reduced binding to an Fc receptor or CD25, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

As used herein, the term “immunoconjugate” refers to a polypeptide molecule that includes at least one IL-2 molecule and at least one antibody. The IL-2 molecule can be joined to the antibody by a variety of interactions and in a variety of configurations as described herein. In particular embodiments, the IL-2 molecule is fused to the antibody via a peptide linker. Particular immunoconjugates according to the invention essentially consist of one IL-2 molecule and an antibody joined by one or more linker sequences.

By “fused” is meant that the components (e.g. an antibody and an IL-2 molecule) are linked by peptide bonds, either directly or via one or more peptide linkers.

As used herein, the terms “first” and “second” with respect to Fc domain subunits etc., are used for convenience of distinguishing when there is more than one of each type of moiety. Use of these terms is not intended to confer a specific order or orientation of the immunoconjugate unless explicitly so stated.

By “specific binding” is meant that the binding is selective for the antigen and can be discriminated from unwanted or non-specific interactions. The ability of an antibody to bind to a specific antigen (e.g. PD-1 or FAP) can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antibody to an unrelated protein is less than about 10% of the binding of the antibody to the antigen as measured, e.g., by SPR.

As used herein, a “peripheral blood mononuclear cells” or “PBMCs” refers to a heterogeneous population of blood cells having a round nucleus. Examples of cells that may be found in a population of PBMCs include lymphocytes such as T cells, B cells, NK cells (including NKT cells and CIK cells) and monocytes such as macrophages and dendritic cells. A “plurality of PBMCs” as used herein refers to a preparation of PBMCs comprising cells of at least two types of blood cells. In some embodiments, a plurality of PBMCs comprises two or more of T cells, B cells, NK cells, macrophages or dendritic cells. In some embodiments, a plurality of PBMCs comprises three or more of T cells, B cells, NK cells, macrophages or dendritic cells. In some embodiments, a plurality of PBMCs comprises four or more of T cells, B cells, NK cells, macrophages or dendritic cells. In some embodiments, a plurality of PBMCs comprises T cells, B cells, NK cells, macrophages and dendritic cells.

PBMCs can be isolated by means known in the art. For example, PBMCs can be derived from peripheral blood of an individual based on density of PBMCs compared to other blood cells. In some embodiments, PBMCs are derived from peripheral blood using a cell size difference and/or a cell density difference based fractionation technique. In some embodiments, PBMCs are derived from peripheral blood of an individual using Ficoll (e.g., a ficoll gradient). In some embodiments, PBMCs are derived from peripheral blood of an individual using ELUTRA® cell separation system.

In some embodiments, a population of PBMCs is isolated from an individual. In some embodiments, a plurality of PBMCs is an autologous population of PBMCs where the population is derived from a particular individual, manipulated by any of the methods described herein, and returned to the particular individual. In some embodiments, a plurality of PBMCs is an allogeneic population of PBMCs where the population is derived from one individual, manipulated by any of the methods described herein, and administered to a second individual.

In some embodiments, a plurality of PBMCs is a reconstituted preparation of PBMCs. In some embodiments, the plurality of PBMCs may be generated by mixing cells typically found in a population of PBMCs; for example, by mixing populations of two or more of T cells, B cells, NK cells, or monocytes. In some embodiments, ratios of cells in a population of splenocytes are adjusted (e.g, crafted) to better reflect the population profile of human PBMCs. For example, B cells may be depleted from a population of splenocytes to better reflect a population of human PBMCs.

The term “pore” as used herein refers to an opening, including without limitation, a hole, tear, cavity, aperture, break, gap, or perforation within a material. In some examples, (where indicated) the term refers to a pore within a surface of the present disclosure. In other examples, (where indicated) a pore can refer to a pore in a cell membrane.

The term “membrane” as used herein refers to a selective barrier or sheet containing pores. The term includes a pliable sheet-like structure that acts as a boundary or lining. In some examples, the term refers to a surface or filter containing pores. This term is distinct from the term “cell membrane”.

The term “filter” as used herein refers to a porous article that allows selective passage through the pores. In some examples, the term refers to a surface or membrane containing pores.

The term “heterogeneous” as used herein refers to something which is mixed or not uniform in structure or composition. In some examples, the term refers to pores having varied sizes, shapes or distributions within a given surface.

The term “homogeneous” as used herein refers to something which is consistent or uniform in structure or composition throughout. In some examples, the term refers to pores having consistent sizes, shapes, or distribution within a given surface.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

The term “heterologous” as it relates to amino acid sequences such as peptide sequences and polypeptide sequences, denotes sequences that are not normally joined together, and/or are not normally associated with a particular cell. Thus, a “heterologous” region of a peptide sequence is a segment of amino acids within or attached to another amino acid molecule that is not found in association with the other molecule in nature. For example, a heterologous region of a peptide construct could include the amino acid sequence of the peptide flanked by sequences not found in association with the amino acid sequence of the peptide in nature. Another example of a heterologous peptide sequence is a construct where the peptide sequence itself is not found in nature (e.g., synthetic sequences having amino acids different as coded from the native gene). Similarly, a cell transformed with a vector that expresses an amino acid construct which is not normally present in the cell would be considered heterologous for purposes of this invention. Allelic variation or naturally occurring mutational events do not give rise to heterologous peptides, as used herein.

The term “exogenous” when used in reference to an agent, such as an antigen or an adjuvant, with relation to a cell refers to an agent delivered into the cell from outside the cell. The cell may or may not have the agent already present, and may or may not produce the agent after the exogenous agent has been delivered.

As used herein, the term “inhibit” may refer to the act of blocking, reducing, eliminating, or otherwise antagonizing the presence, or an activity of, a particular target. Inhibition may refer to partial inhibition or complete inhibition. For example, inhibiting an immune response may refer to any act leading to a blockade, reduction, elimination, or any other antagonism of an immune response. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “suppress” may refer to the act of decreasing, reducing, prohibiting, limiting, lessening, or otherwise diminishing the presence, or an activity of, a particular target. Suppression may refer to partial suppression or complete suppression. For example, suppressing an immune response may refer to any act leading to decreasing, reducing, prohibiting, limiting, lessening, or otherwise diminishing an immune response. In other examples, suppression of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “enhance” may refer to the act of improving, boosting, heightening, or otherwise increasing the presence, or an activity of, a particular target. For example, enhancing an immune response may refer to any act leading to improving, boosting, heightening, or otherwise increasing an immune response. In one exemplary example, enhancing an immune response may refer to employing an antigen and/or adjuvant to improve, boost, heighten, or otherwise increase an immune response. In other examples, enhancing the expression of a nucleic acid may include, but not limited to increase in the transcription of a nucleic acid, increase in mRNA abundance (e.g., increasing mRNA transcription), decrease in degradation of mRNA, increase in mRNA translation, and so forth.

As used herein, the term “modulate” may refer to the act of changing, altering, varying, or otherwise modifying the presence, or an activity of, a particular target. For example, modulating an immune response may refer to any act leading to changing, altering, varying, or otherwise modifying an immune response. In other examples, modulating the expression of a nucleic acid may include, but not limited to a change in the transcription of a nucleic acid, a change in mRNA abundance (e.g., increasing mRNA transcription), a corresponding change in degradation of mRNA, a change in mRNA translation, and so forth.

As used herein, the term “induce” may refer to the act of initiating, prompting, stimulating, establishing, or otherwise producing a result. For example, inducing an immune response may refer to any act leading to initiating, prompting, stimulating, establishing, or otherwise producing a desired immune response. In other examples, inducing the expression of a nucleic acid may include, but not limited to initiation of the transcription of a nucleic acid, initiation of mRNA translation, and so forth.

The term “homologous” as used herein refers to a molecule which is derived from the same organism. In some examples, the term refers to a nucleic acid or protein which is normally found or expressed within the given organism.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and phosphorothioates, and thus can be an oligodeoxynucleoside phosphoramidate (P-NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

As used herein, the term “adjuvant” refers to a substance which either directly or indirectly modulates and/or engenders an immune response. Generally, the adjuvant is administered in conjunction with an antigen to effect enhancement of an immune response to the antigen as compared to antigen alone. In some embodiments, an adjuvant is used to condition a plurality of PBMCs (e.g., as demonstrated in the Examples). Various adjuvants are described herein.

The terms “CpG oligodeoxynucleotide” and “CpG ODN” refer to DNA molecules containing a dinucleotide of cytosine and guanine separated by a phosphate (also referred to herein as a “CpG” dinucleotide, or “CpG”). The CpG ODNs of the present disclosure contain at least one unmethylated CpG dinucleotide. That is, the cytosine in the CpG dinucleotide is not methylated (i.e., is not 5-methylcytosine). CpG ODNs may have a partial or complete phosphorothioate (PS) backbone.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. The methods of the invention contemplate any one or more of these aspects of treatment.

As used herein, by “combination therapy” is meant that a first agent be administered in conjunction with another agent. “In conjunction with” refers to administration of one treatment modality in addition to another treatment modality, such as administration of a composition of nucleated cells as described herein in addition to administration of an immunoconjugate as described herein to the same individual. As such, “in conjunction with” refers to administration of one treatment modality before, during, or after delivery of the other treatment modality to the individual.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in the cancer. In some embodiments, an effective amount is an amount sufficient to delay development of cancer. In some embodiments, an effective amount is an amount sufficient to prevent or delay recurrence. An effective amount can be administered in one or more administrations. In the case of cancer, the effective amount of the composition or immunoconjugate may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) relieve to some extent one or more of the symptoms associated with cancer; and/or (viii) disrupting (such as destroying) cancer stroma.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, the term “prophylactic treatment” refers to treatment, wherein an individual is known or suspected to have or be at risk for having a disorder but has displayed no symptoms or minimal symptoms of the disorder. An individual undergoing prophylactic treatment may be treated prior to onset of symptoms.

A “therapeutically effective amount” of an agent, e.g. a pharmaceutical composition, refers to an amount effective, at dosages and for periods of time necessary, to achieve the desired therapeutic result. A therapeutically effective amount of an agent for example eliminates, decreases, delays, minimizes or prevents adverse effects of a disease.

A “prophylactically effective amount” of an agent, e.g. a pharmaceutical composition, refers to an amount effective, at dosages and for periods of time necessary, to achieve the desired prophylactic result. A prophylactically effective amount of an agent, for example, may prevent the disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the composition would be administered.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

For any of the structural and functional characteristics described herein, methods of determining these characteristics are known in the art.

### Mutant IL-2 Polypeptide

The immunoconjugates according to the present invention comprise a mutant IL-2 polypeptide having advantageous properties for immunotherapy. In particular, pharmacological properties of IL-2 that contribute to toxicity but are not essential for efficacy of IL-2 are eliminated in the mutant IL-2 polypeptide. Such mutant IL-2 polypeptides are described in detail in WO 2012/107417, which is incorporated herein by reference in its entirety. Different forms of the IL-2 receptor consist of different subunits and exhibit different affinities for IL-2. The intermediate-affinity IL-2 receptor, consisting of the β and γ receptor subunits, is expressed on resting effector cells and is sufficient for IL-2 signaling. The high-affinity IL-2 receptor, additionally comprising the α-subunit of the receptor (CD25), is mainly expressed on regulatory T (Treg) cells as well as on activated effector cells where its engagement by IL-2 can promote Treg cell-mediated immunosuppression or activation-induced cell death (AICD), respectively. Thus, without wishing to be bound by theory, reducing or abolishing the affinity of IL-2 to the α-subunit of the IL-2 receptor should reduce IL-2 induced downregulation of effector cell function by regulatory T cells and development of tumor tolerance by the process of AICD. On the other hand, maintaining the affinity to the intermediate-affinity IL-2 receptor should preserve the induction of proliferation and activation of effector cells like NK and T cells by IL-2. In some embodiments, the IL-2 polypeptide of the immunoconjugate comprises the amino acid substitutions F42A, Y45A and L72G (numbering relative to the human IL-2 sequence SEQ ID NO: 19).

Additional amino acid substitutions of human IL-2 (hIL-2) that may decrease affinity to CD25 may for example be generated at amino acid position 35, 38, or 43 or combinations thereof (numbering relative to the human IL-2 sequence SEQ ID NO: 19) and combinations with F42A, Y45A and L72G. Exemplary amino acid substitutions include K35E, K35A, R38A, R38E, R38N, R38F, R38S, R38L, R38G, R38Y, R38W, and K43E. These mutants exhibit substantially similar binding affinity to the intermediate-affinity IL-2 receptor, and have substantially reduced affinity to the α-subunit of the IL-2 receptor and the high-affinity IL-2 receptor compared to a wild-type form of the IL-2 mutant.

Other characteristics of useful mutants may include the ability to induce proliferation of IL-2 receptor-bearing T and/or NK cells, the ability to induce IL-2 signaling in IL-2 receptor-bearing T and/or NK cells, the ability to generate interferon (IFN)-γ as a secondary cytokine by NK cells, a reduced ability to induce elaboration of secondary cytokines—particularly IL-10 and TNF-α—by peripheral blood mononuclear cells (PBMCs), a reduced ability to activate regulatory T cells, a reduced ability to induce apoptosis in T cells, and a reduced toxicity profile in vivo.

In certain embodiments said amino acid mutation reduces the affinity of the mutant IL-2 polypeptide to the α-subunit of the IL-2 receptor by at least 5-fold, specifically at least 10-fold, more specifically at least 25-fold. In embodiments where there is more than one amino acid mutation that reduces the affinity of the mutant IL-2 polypeptide to the α-subunit of the IL-2 receptor, the combination of these amino acid mutations may reduce the affinity of the mutant IL-2 polypeptide to the α-subunit of the IL-2 receptor by at least 30-fold, at least 50-fold, or even at least 100-fold. In one embodiment said amino acid mutation or combination of amino acid mutations abolishes the affinity of the mutant IL-2 polypeptide to the α-subunit of the IL-2 receptor so that no binding is detectable by surface plasmon resonance.

Substantially similar binding to the intermediate-affinity receptor, i.e. preservation of the affinity of the mutant IL-2 polypeptide to said receptor, is achieved when the IL-2 mutant exhibits greater than about 70% of the affinity of a wild-type form of the IL-2 mutant to the intermediate-affinity IL-2 receptor. IL-2 mutants of the invention may exhibit greater than about 80% and even greater than about 90% of such affinity.

Reduction of the affinity of IL-2 for the α-subunit of the IL-2 receptor in combination with elimination of the O-glycosylation of IL-2 results in an IL-2 protein with improved properties. For example, elimination of the O-glycosylation site results in a more homogenous product when the mutant IL-2 polypeptide is expressed in mammalian cells such as CHO or HEK cells.

Thus, in certain embodiments the mutant IL-2 polypeptide comprises an additional amino acid mutation which eliminates the O-glycosylation site of IL-2 at a position corresponding to residue 3 of human IL-2. In one embodiment said additional amino acid mutation which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P. In a specific embodiment, said additional amino acid mutation is the amino acid substitution T3A.

In certain embodiments, the mutant IL-2 polypeptide is essentially a full-length IL-2 molecule and comprises the amino acid substitutions F42A, Y45A and L72G (numbering relative to the human IL-2 sequence SEQ ID NO:19). In certain embodiments, the mutant IL-2 polypeptide is a human IL-2 molecule. In a specific embodiment, the mutant IL-2 polypeptide comprising the amino acid substitutions F42A, Y45A and L72G (numbering relative to the human IL-2 sequence SEQ ID NO:19) can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In one embodiment the mutant IL-2 polypeptide comprising the amino acid substitutions F42A, Y45A and L72G (numbering relative to the human IL-2 sequence SEQ ID NO: 19) has a reduced ability to induce IL-2 signaling in regulatory T cells, compared to a wild-type IL-2 polypeptide. In one embodiment the mutant IL-2 polypeptide induces less activation-induced cell death (AICD) in T cells, compared to a wild-type IL-2 polypeptide. In one embodiment, the mutant IL-2 polypeptide has a reduced toxicity profile in vivo, compared to a wild-type IL-2 polypeptide. In one embodiment, the mutant IL-2 polypeptide has a prolonged serum half-life, compared to a wild-type IL-2 polypeptide.

A particular mutant IL-2 polypeptide useful in the invention comprises four amino acid substitutions at positions corresponding to residues 3, 42, 45 and 72 of human IL-2. Specific amino acid substitutions are T3A, F42A, Y45A and L72G. As demonstrated in WO 2012/107417, said quadruple mutant IL-2 polypeptide exhibits no detectable binding to CD25, reduced ability to induce apoptosis in T cells, reduced ability to induce IL-2 signaling in Treg cells, and a reduced toxicity profile in vivo. However, it retains ability to activate IL-2 signaling in effector cells, to induce proliferation of effector cells, and to generate IFN-γ as a secondary cytokine by NK cells.

Moreover, said mutant IL-2 polypeptide has further advantageous properties, such as reduced surface hydrophobicity, good stability, and good expression yield, as described in WO 2012/107417. Unexpectedly, said mutant IL-2 polypeptide also provides a prolonged serum half-life, compared to wild-type IL-2.

IL-2 mutants useful in the invention, in addition to having mutations in the region of IL-2 that forms the interface of IL-2 with CD25 or the glycosylation site, also may have one or more mutations in the amino acid sequence outside these regions. Such additional mutations in human IL-2 may provide additional advantages such as increased expression or stability. For example, the cysteine at position 125 may be replaced with a neutral amino acid such as serine, alanine, threonine or valine, yielding C125S IL-2, C125A IL-2, C125T IL-2 or C125V IL-2 respectively, as described in U.S. Pat. No. 4,518,584. As described therein, one may also delete the N-terminal alanine residue of IL-2 yielding such mutants as des-A1 C125S or des-A1 C125A. Alternatively or conjunctively, the IL-2 mutant may include a mutation whereby methionine normally occurring at position 104 of wild-type human IL-2 is replaced by a neutral amino acid such as alanine (see U.S. Pat. No. 5,206,344). The resulting mutants, e. g., des-A1 M104A IL-2, des-A1 M104A C125S IL-2, M104A IL-2, M104A C125A IL-2, des-A1 M104A C125A IL-2, or M104A C125S IL-2 (these and other mutants may be found in U.S. Pat. No. 5,116,943 and in Weiger et al., Eur J Biochem 180, 295-300 (1989)) may be used in conjunction with the particular IL-2 mutations of the invention.

Thus, in certain embodiments the mutant IL-2 polypeptide comprises an additional amino acid mutation at a position corresponding to residue 125 of human IL-2. In one embodiment, said additional amino acid mutation is the amino acid substitution C125A.

The skilled person will be able to determine which additional mutations may provide additional advantages for the purpose of the invention. For example, he will appreciate that amino acid mutations in the IL-2 sequence that reduce or abolish the affinity of IL-2 to the intermediate-affinity IL-2 receptor, such as D20T, N88R or Q126D (see e.g. US 2007/0036752), may not be suitable to include in the mutant IL-2 polypeptide according to the invention.

In one embodiment, the mutant IL-2 polypeptide comprises no more than 12, no more than 11, no more than 10, no more than 9, no more than 8, no more than 7, no more than 6, or no more than 5 amino acid mutations as compared to the corresponding wild-type IL-2 sequence, e.g. the human IL-2 sequence of SEQ ID NO:19. In a particular embodiment, the mutant IL-2 polypeptide comprises no more than 5 amino acid mutations as compared to the corresponding wild-type IL-2 sequence, e.g. the human IL-2 sequence of SEQ ID NO: 19.

In one embodiment, the mutant IL-2 polypeptide comprises the sequence of SEQ ID NO:20. In one embodiment, the mutant IL-2 polypeptide consists of the sequence of SEQ ID NO:20.

In some embodiments, the invention provides an immunoconjugate comprising a mutant interleukin-2 (IL-2) polypeptide comprises an amino acid sequence that is at least about 95%, 96%, 97%, 98%, 99% or 100% identical to the amino acid sequence of SEQ ID NO:20. In some embodiments, the invention provides an immunoconjugate comprising a mutant interleukin-2 (IL-2) polypeptide comprises an amino acid sequence that is at least about 95%, 96%, 97%, 98%, 99% or 100% identical to the amino acid sequence of SEQ ID NO:20, wherein the IL-2 polypeptide displays reduced affinity of the mutant IL-2 polypeptide to the high-affinity IL-2 receptor and preserves affinity of the mutant IL-2 polypeptide to the intermediate-affinity IL-2 receptor, each compared to a wild-type IL-2 polypeptide. In some embodiments, the invention provides an immunoconjugate comprising a mutant interleukin-2 (IL-2) polypeptide comprising at least one amino acid mutation that abolishes or reduces affinity of the mutant IL-2 polypeptide to the high-affinity IL-2 receptor and preserves affinity of the mutant IL-2 polypeptide to the intermediate-affinity IL-2 receptor, each compared to a wild-type IL-2 polypeptide. In some embodiments, the invention provides an immunoconjugate comprising a mutant interleukin-2 (IL-2) polypeptide comprising at least one amino acid mutation, wherein the mutant IL-2 has a lower (or abolished) affinity to the high-affinity IL-2 receptor and a higher (or equal) affinity to the intermediate affinity IL-2 receptor, each compared to a mutant IL-2 polypeptide consisting of the sequence of SEQ ID NO:20.

### Immunoconjugates

Immunoconjugates as described herein comprise an IL-molecule and a second polypeptide, wherein the second polypeptide is capable of specific binding to a T cell, a tumor cell, or the tumor cell environment. Such immunoconjugates significantly increase the efficacy of IL-2 therapy by directly targeting IL-2 e.g. into a tumor microenvironment. In some embodiments, an antigen-binding moiety comprised in the immunoconjugate can be a whole antibody or immunoglobulin, or a portion or variant thereof that has a biological function such as antigen specific binding affinity.

In some embodiments, an antigen-binding moiety comprised in an immunoconjugate polypeptide is capable of specific binding to a T cell, a tumor cell, or the tumor cell environment and results in targeting of the immunoconjugate molecule to the tumor site. Therefore, high concentrations of IL-2 can be delivered into the tumor microenvironment, thereby resulting in activation and proliferation of a variety of immune effector cells mentioned herein using a much lower dose of the immunoconjugate than would be required for unconjugated IL-2. Moreover, since application of IL-2 in form of immunoconjugates allows lower doses of the cytokine itself, the potential for undesirable side effects of IL-2 is restricted, and targeting the IL-2 to a specific site in the body by means of an immunoconjugate may also result in a reduction of systemic exposure and thus less side effects than obtained with unconjugated IL-2. In addition, the increased circulating half-life of an immunoconjugate compared to unconjugated IL-2 contributes to the efficacy of the immunoconjugate. However, this characteristic of IL-2 immunoconjugates may again aggravate potential side effects of the IL-2 molecule: Because of the significantly longer circulating half-life of IL-2 immunoconjugate in the bloodstream relative to unconjugated IL-2, the probability for IL-2 or other portions of the fusion protein molecule to activate components generally present in the vasculature is increased. The same concern applies to other fusion proteins that contain IL-2 fused to another moiety such as Fc or albumin, resulting in an extended half-life of IL-2 in the circulation. Therefore an immunoconjugate comprising a mutant TL-2 polypeptide as described herein and in WO 2012/107417, with reduced toxicity compared to wild-type forms of IL-2, is particularly advantageous.

As described hereinabove, targeting IL-2 directly to immune effector cells rather than tumor cells may be advantageous for IL-2 immunotherapy.

Accordingly, in some embodiments, the invention provides a mutant IL-2 polypeptide as described hereinbefore, and an antigen-binding moiety that binds to PD-1. In one embodiment, the mutant IL-2 polypeptide and the PD-1 antigen-binding moiety form a fusion protein, i.e. the mutant IL-2 polypeptide shares a peptide bond with the PD-1 antigen-binding moiety. In some embodiments, the PD-1 antigen-binding moiety comprises an Fc domain composed of a first and a second subunit. In a specific embodiment, the mutant IL-2 polypeptide is fused at its amino-terminal amino acid to the carboxy-terminal amino acid of one of the subunits of the Fc domain, optionally through a linker peptide. In some embodiments, the PD-1 antigen-binding moiety is a full-length antibody. In some embodiments, the antibody is an immunoglobulin molecule, particularly an IgG class immunoglobulin molecule, more particularly an IgG1 subclass immunoglobulin molecule. In one such embodiment, the mutant IL-2 polypeptide shares an amino-terminal peptide bond with one of the immunoglobulin heavy chains. In certain embodiments, the PD-1 antigen-binding moiety is an antibody fragment. In some embodiments, the PD-1 antigen-binding moiety is a Fab molecule or a scFv molecule. In one embodiment, the PD-1 antigen-binding moiety is a Fab molecule. In another embodiment, the PD-1 antigen-binding moiety is a scFv molecule. The immunoconjugate may also comprise more than one antigen-binding moieties. Where more than one antigen-binding moiety is comprised in the immunoconjugate, e.g. a first and a second antibody, each antibody can be independently selected from various forms of antibodies and antibody fragments. For example, the first antibody can be a Fab molecule and the second antibody can be a scFv molecule. In a specific embodiment, each of said first and said second antibodies is a scFv molecule or each of said first and said second antibodies is a Fab molecule. In a particular embodiment, each of said first and said second antibodies is a Fab molecule. In one embodiment, each of said first and said second antibodies binds to PD-1.

In some embodiments, the invention provides a mutant IL-2 polypeptide as described hereinbefore, and an antigen-binding moiety that specifically binds a target antigen presented on a tumor cell or in a tumor cell environment. In some embodiments, the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP).

In some embodiments, the invention provides a mutant IL-2 polypeptide as described hereinbefore, and an antigen-binding moiety that binds to FAP. In one embodiment, the mutant IL-2 polypeptide and the FAP antigen-binding moiety form a fusion protein, i.e. the mutant IL-2 polypeptide shares a peptide bond with the FAP antigen-binding moiety. In some embodiments, the FAP antigen-binding moiety comprises an Fc domain composed of a first and a second subunit. In a specific embodiment, the mutant IL-2 polypeptide is fused at its amino-terminal amino acid to the carboxy-terminal amino acid of one of the subunits of the Fc domain, optionally through a linker peptide. In some embodiments, the FAP antigen-binding moiety is a full-length antibody. In some embodiments, the antibody is an immunoglobulin molecule, particularly an IgG class immunoglobulin molecule, more particularly an IgG1 subclass immunoglobulin molecule. In one such embodiment, the mutant IL-2 polypeptide shares an amino-terminal peptide bond with one of the immunoglobulin heavy chains. In certain embodiments, the FAP antigen-binding moiety is an antibody fragment. In some embodiments, the FAP antigen-binding moiety is a Fab molecule or a scFv molecule. In one embodiment, the FAP antigen-binding moiety is a Fab molecule. In another embodiment, the FAP antigen-binding moiety is a scFv molecule. The immunoconjugate may also comprise more than one antigen-binding moieties. Where more than one antigen-binding moiety is comprised in the immunoconjugate, e.g. a first and a second antibody, each antibody can be independently selected from various forms of antibodies and antibody fragments. For example, the first antibody can be a Fab molecule and the second antibody can be a scFv molecule. In a specific embodiment, each of said first and said second antibodies is a scFv molecule or each of said first and said second antibodies is a Fab molecule. In a particular embodiment, each of said first and said second antibodies is a Fab molecule. In one embodiment, each of said first and said second antibodies binds to FAP.

### Immunoconjugate Formats

An exemplary immunoconjugate format is described in PCT Publication No. WO 2018/184964, which is incorporated herein by reference in its entirety. In particular embodiments, the immunoconjugate comprises a mutant IL-2 polypeptide as described herein and a second polypeptide. In some embodiments, the second polypeptide is capable of specific binding to a T cell, a tumor cell, or the tumor cell environment. In some embodiments, the second polypeptide binds a T cell a tumor cell or binds an antigen in the tumor cell environment.

In some embodiments, the second polypeptide binds PD-1 expressed on a T cell. In some embodiments, the second polypeptide is an antigen-binding moiety that specifically binds PD-1.

In some embodiments, wherein the second polypeptide specifically binds a target antigen presented on a tumor cell or in a tumor cell environment. In some embodiments, the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP). In some embodiments, the tumor antigen is a FAP. In some embodiments, the second polypeptide binds FAP. In some embodiments, the second polypeptide is an antigen-binding moiety that specifically binds FAP. In some embodiments, the antigen-binding moiety is an immunoglobulin, particularly an IgG molecule, more particularly an IgG1 molecule. In one embodiment, the immunoconjugate comprises not more than one mutant IL-2 polypeptide. In one embodiment, the immunoglobulin molecule is human. In one embodiment, the immunoglobulin molecule comprises a human constant region, e.g. a human CH1, CH2, CH3 and/or CL domain. In one embodiment, the immunoglobulin comprises a human Fc domain, particularly a human IgG1 Fc domain. In one embodiment, the mutant IL-2 polypeptide shares an amino- or carboxy-terminal peptide bond with the immunoglobulin molecule. In one embodiment, the immunoconjugate essentially consists of a mutant IL-2 polypeptide and an immunoglobulin molecule, particularly an IgG molecule, more particularly an IgG1 molecule, joined by one or more linker sequences. In a specific embodiment, the mutant IL-2 polypeptide is fused at its amino-terminal amino acid to the carboxy-terminal amino acid of one of the immunoglobulin heavy chains, optionally through a linker peptide.

The mutant IL-2 polypeptide may be fused to the antibody directly or through a linker peptide, comprising one or more amino acids, typically about 2-20 amino acids. Linker peptides are known in the art and are described herein. Suitable, non-immunogenic linker peptides include, for example, (G₄S)ₙ, (SG₄)ₙ, (G₄S)ₙ or G₄(SG₄)ₙ linker peptides. “n” is generally an integer from 1 to 10, typically from 2 to 4. In one embodiment, the linker peptide has a length of at least 5 amino acids, in one embodiment a length of 5 to 100, in a further embodiment of 10 to 50 amino acids. In a particular embodiment, the linker peptide has a length of 15 amino acids. In one embodiment the linker peptide is (GₓS)ₙ or (GₓS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=3. In a particular embodiment, the linker peptide is (G₄S)3 (SEQ ID NO:21). In one embodiment, the linker peptide has (or consists of) the amino acid sequence of SEQ ID NO:21.

In a particular embodiment, the immunoconjugate comprises a mutant IL-2 molecule and an antigen-binding moiety, wherein the antigen-binding moiety is an immunoglobulin molecule, particularly an IgG1 subclass immunoglobulin molecule, that binds to PD-1, wherein the mutant IL-2 molecule is fused at its amino-terminal amino acid to the carboxy-terminal amino acid of one of the immunoglobulin heavy chains through the linker peptide of SEQ ID NO:21.

In a particular embodiment, the immunoconjugate comprises a mutant IL-2 molecule and an antigen-binding moiety that binds to PD-1, wherein the antigen-binding moiety comprises an Fc domain, particularly a human IgG1 Fc domain, composed of a first and a second subunit, and the mutant IL-2 molecule is fused at its amino-terminal amino acid to the carboxy-terminal amino acid of one of the subunits of the Fc domain through the linker peptide of SEQ ID NO:21.

In a particular embodiment, the immunoconjugate comprises a mutant IL-2 molecule and an antigen-binding moiety wherein the antigen-binding moiety is an immunoglobulin molecule, particularly an IgG1 subclass immunoglobulin molecule, that binds to FAP, wherein the mutant IL-2 molecule is fused at its amino-terminal amino acid to the carboxy-terminal amino acid of one of the immunoglobulin heavy chains through the linker peptide of SEQ ID NO:21.

In a particular embodiment, the immunoconjugate comprises a mutant IL-2 molecule and an antigen-binding moiety that binds to FAP, wherein the antigen-binding moiety comprises an Fc domain, particularly a human IgG1 Fc domain, composed of a first and a second subunit, and the mutant IL-2 molecule is fused at its amino-terminal amino acid to the carboxy-terminal amino acid of one of the subunits of the Fc domain through the linker peptide of SEQ ID NO:21. In some embodiments, the immunoconjugate is simlukafusp alfa.

### PD-1 Antigen-Binding Moieties

In some embodiments, the immunoconjugate comprises a mutant IL-2 polypeptide and PD-1 antigen-binding moiety. In some embodiments, the PD-1 antigen-binding moiety is an anti-PD-1 antibody.

The PD-1 antigen-binding moiety comprised in the immunoconjugate of the invention binds to PD-1, particularly human PD-1, and is able to direct the mutant IL-2 polypeptide to a target site where PD-1 is expressed, particularly to a T cell that expresses PD-1; for example, a T cell associated with a tumor or a T cell capable of binding to a tumor antigen.

Suitable PD-1 antibodies that may be used in the immunoconjugate of the invention are described in PCT patent application no. PCT/EP2016/073248, which is incorporated herein by reference in its entirety.

The immunoconjugate of the invention may comprise two or more PD-1 antigen-binding moieties, which may bind to the same or to different antigens. In one embodiment, the antigen-binding moiety comprised in the immunoconjugate of the invention is monospecific. In a particular embodiment, the immunoconjugate comprises a single, monospecific antibody, particularly a monospecific immunoglobulin molecule.

The antigen-binding moiety can be any type of antibody or fragment thereof that retains specific binding to PD-1, particularly human PD-1. Antibody fragments include, but are not limited to, Fv molecules, scFv molecule, Fab molecule, and F(ab′)2 molecules. In particular embodiments, however, the antibody is a full-length antibody. In some embodiments, the antibody comprises an Fc domain, composed of a first and a second subunit. In some embodiments, the antibody is an immunoglobulin, particularly an IgG class, more particularly an IgG1 subclass immunoglobulin.

In some embodiments, the antibody is a monoclonal antibody.

In some embodiments, the PD-1 antigen-binding moiety comprises a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, a FR-H3 comprising the amino acid sequence of SEQ ID NO:7 at positions 71-73 according to Kabat numbering, a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6.

In some embodiments, the PD-1 antigen-binding moiety comprises (a) a heavy chain variable region (VH) comprising a HVR-H1 comprising the amino acid sequence of SEQ ID NO:1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and (b) a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6. In some embodiments, the heavy and/or light chain variable region is a humanized variable region. In some embodiments, the heavy and/or light chain variable region comprises human framework regions (FR).

In some embodiments, the PD-1 antigen-binding moiety comprises a HVR-H1 comprising the amino acid sequence of SEQ ID NO:8, a HVR-H2 comprising the amino acid sequence of SEQ ID NO:9, a HVR-H3 comprising the amino acid sequence of SEQ ID NO: 10, a HVR-L1 comprising the amino acid sequence of SEQ ID NO:11, a HVR-L2 comprising the amino acid sequence of SEQ ID NO: 12, and a HVR-L3 comprising the amino acid sequence of SEQ ID NO:13.

In some embodiments, the PD-1 antigen-binding moiety comprises (a) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and (b) a light chain variable region (VL) comprising a HVR-L1 comprising the amino acid sequence of SEQ ID NO:11, a HVR-L2 comprising the amino acid sequence of SEQ ID NO: 12, and a HVR-L3 comprising the amino acid sequence of SEQ ID NO:13. In some embodiments, the heavy and/or light chain variable region is a humanized variable region. In some embodiments, the heavy and/or light chain variable region comprises human framework regions (FR).

In some embodiments, the PD-1 antigen-binding moiety comprises a heavy chain variable region (VH) comprising an amino acid sequence that is at least about 95%, 96%, 97%, 98%, 99% or 100% identical to the amino acid sequence of SEQ ID NO:14. In some embodiments, the PD-1 antigen-binding moiety comprises a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO: 17, and SEQ ID NO: 18. In some embodiments, the PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO: 14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO: 17, and SEQ ID NO:18. In some embodiments, the PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO: 14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO: 17, and SEQ ID NO: 18, wherein the PD-1 antigen-binding moiety specifically binds PD-1.

In a particular embodiment, the PD-1 antigen-binding moiety comprises (a) a heavy chain variable region (VH) comprising the amino acid sequence of SEQ ID NO: 14, and (b) a light chain variable region (VL) comprising the amino acid sequence of SEQ ID NO: 15.

In some embodiments, the PD-1 antigen-binding moiety is a humanized antibody. In one embodiment, the antibody is an immunoglobulin molecule comprising a human constant region, particularly an IgG class immunoglobulin molecule comprising a human CH1, CH2, CH3 and/or CL domain. Exemplary sequences of human constant domains human kappa and lambda CL domains and human IgG1 heavy chain constant domains CH1-CH2-CH3. Particularly, the heavy chain constant region may comprise amino acid mutations in the Fc domain as described herein.

### FAP Antigen-Binding Moieties

In some embodiments, the immunoconjugate comprises a mutant IL-2 polypeptide and an FAP antigen-binding moiety. The antibody comprised in the immunoconjugate of the invention binds to FAP, particularly human FAP, and is able to direct the mutant IL-2 polypeptide to a target site where FAP is expressed; for example, a tumor cell that expresses FAP. An immunoconjugate comprising a mutant IL-2 polypeptide and a FAP antigen-binding domain is described in EP3075745B1.

In some embodiments, the antigen-binding moiety that specifically bind FAP comprising (i) the heavy chain variable region sequence of SEQ ID NO:29 and the light chain variable region sequence of SEQ ID NO:28; (ii) the heavy chain variable region sequence of SEQ ID NO:31 and the light chain variable region sequence of SEQ ID NO:30; (iii) the heavy chain variable region sequence of SEQ ID NO:33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36.

In some embodiments, the antigen-binding moiety that specifically bind FAP comprising (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO:40; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41.

In some embodiments, the antigen-binding moiety that specifically bind FAP comprises the heavy chain variable region sequence of SEQ ID NO:33 and the light chain variable region sequence of SEQ ID NO:32. In some embodiments, the immunoconjugate comprises a polypeptide sequence of SEQ ID NO:38, a polypeptide sequence of SEQ ID NO:39, and a polypeptide sequence of SEQ ID NO:40.

In some embodiments, the immunoconjugate comprises a heavy chain of a PD-1 binding moiety fused to IL2v comprising an amino acid sequence of SEQ ID NO:22, a PD-1 binding moiety heavy chain comprising an amino acid sequence of SEQ ID NO:24 and two PD-1 binding moiety light chains comprising an amino acid sequence of SEQ ID NO:25. In some embodiments, the immunoconjugate comprises a heavy chain of a FAP binding moiety fused to IL2v comprising an amino acid sequence of SEQ ID NO:38, a FAP binding moiety heavy chain comprising an amino acid sequence of SEQ ID NO:39 and two FAP binding moiety light chains comprising an amino acid sequence of SEQ ID NO:40. In some embodiments, the immunoconjugate is simlukafusp alfa.

### Fc Domain

In particular embodiments, the antibody comprised in the immunoconjugates according to the invention comprises an Fc domain, composed of a first and a second subunit. The Fc domain of an antibody consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment, the immunoconjugate of the invention comprises not more than one Fc domain.

In one embodiment, the Fc domain of the antibody comprised in the immunoconjugate is an IgG Fc domain. In a particular embodiment, the Fc domain is an IgG1 Fc domain. In another embodiment, the Fc domain is an IgG4 Fc domain. In a more specific embodiment, the Fc domain is an IgG4 Fc domain comprising an amino acid substitution at position S228 (Kabat EU index numbering), particularly the amino acid substitution S228P. This amino acid substitution reduces in vivo Fab arm exchange of IgG4 antibodies (see Stubenrauch et al., Drug Metabolism and Disposition 38, 84-91 (2010)). In a further particular embodiment, the Fc domain is a human Fc domain. In an even more particular embodiment, the Fc domain is a human IgG1 Fc domain.

### Fc Domain Modifications Promoting Heterodimerization

Immunoconjugates according to the invention comprise a mutant IL-2 polypeptide, particularly a single (not more than one) mutant IL-2 polypeptide, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of the immunoconjugate in recombinant production, it will thus be advantageous to introduce in the Fc domain of the antibody a modification promoting the association of the desired polypeptides.

Accordingly, in particular embodiments, the Fc domain of the antibody comprised in the immunoconjugate according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed).

In a specific embodiment, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

### Compositions of Nucleated Cells

In some embodiments, the methods of the invention provide for the administration to an individual in need thereof an effective amount of compositions of nucleated cells comprising an exogenous antigen in conjunction with administration of an effective amount of an immunoconjugate comprising a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19). In some embodiments, the composition of nucleated cells is a composition of immune cells. In some embodiments, the composition of nucleated cells comprises a plurality of PBMCs. In some embodiments, the nucleated cells are one or more of T cells, B cells, NK cells, monocytes, dendritic cells and/or NK-T cells.

In a particular embodiment of the invention, the nucleated cells comprising an exogenous antigen of the composition are PBMCs. As used herein, PBMCs may be isolated by leukapheresis from whole blood obtained from an individual. Also provided are PBMC compositions reconstituted by mixing different pools of PBMCs from the same individual or different individuals. In other examples, PBMCs may also be reconstituted by mixing different populations of cells into a mixed cell composition with a generated profile. In some embodiments, the populations of cells used for reconstituting PBMCs are mixed populations of cells (such as a mixture of one or more of T cells, B cells, NK cells or monocytes). In some embodiments, the populations of cells used for reconstituting PBMCs are purified populations of cells (such as purified T cells, B cells, NK cells or monocytes). In additional examples, the different populations of cells used in reconstituting a PBMC composition can be isolated from the same individual (e.g. autologous) or isolated from different individuals (e.g. allogenic and/or heterologous).

Therefore, in some embodiments according to the methods described herein, the plurality of PBMCs comprises one or more of T cells, B cells, NK cells, monocytes, dendritic cells or NK-T cells. In some embodiments, the plurality of PBMCs comprises T cells, B cells, NK cells, monocytes, dendritic cells or NK-T cells. In some embodiments, the plurality of PBMCs comprises one or more of CD3+ T cells, CD20+ B cells, CD14+ monocytes, CD56+NK cells. In some embodiments, the plurality of PBMCs comprises T cells, B cells, NK cells and monocytes, and the ratio of T cells, B cells, NK cells and monocytes to the total number of PBMCs in the plurality of PBMCs is essentially the same as the ratio of T cells, B cells, NK cells and monocytes to the total number of PBMCs in whole blood. In some embodiments, the plurality of PBMCs comprises T cells, B cells, NK cells and monocytes, and the ratio of T cells, B cells, NK cells and monocytes to the total number of PBMCs in the plurality of PBMCs is essentially the same as the ratio of T cells, B cells, NK cells and monocytes to the total number of PBMCs in a leukapheresis product from whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 1%, 2%, 5%, 10% 15%, 20%, 25%, 30%, 40%, or 50% from the ratio of T cells, B cells, NK cells and monocytes to the total number of PBMCs in whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 10% from the ratio of T cells, B cells, NK cells and monocytes to the total number of PBMCs in whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 1%, 2%, 5%, 10% 15%, 20%, 25%, 30%, 40%, or 50% from the ratio of T cells, B cells, NK cells and monocytes to the total number of PBMCs in a leukapheresis product from whole blood. In some embodiments, the plurality of PBMCs comprises T cells, B cells, NK cells and monocytes, and the ratio of T cells, B cells, NK cells and monocytes to the total number of PBMCs in the plurality of PBMCs differs by not more than any one of 10% from the ratio of T cells, B cells, NK cells and monocytes to the total number of PBMCs in a leukapheresis product from whole blood.

In some embodiments according to the methods described herein, about 25% to about 70% of the modified PBMCs are T cells. In some embodiments, about 2.5% to about 14% of the modified PBMCs are B cells. In some embodiments, about 3.5% to about 35% of the modified PBMCs are NK cells. In some embodiments, about 4% to about 25% of the modified PBMCs are NK cells.

In some embodiments according to the methods described herein, at least about any one of 15%, 20%, 25%, 30%, 35%, 40%, 45%, 50%, 55%, 60%, 65%, 70%, or 75% of the PBMCs are T cells. In some embodiments, at least about 25% of the PBMCs are T cells. In some embodiments, at least about any one of 0.5%, 1%, 1.5%, 2%, 2.5%, 3%, 4%, 5%, 6%, 7%, 7.5%, 8%, 9%, 10%, 11%, 12%, 13%, 14%, 15%, 16%, 17%, 18%, 19%, 20%, 25%, or 30% of the PBMCs are B cells. In some embodiments, at least about 2.5% of the PBMCs are B cells. In some embodiments, at least about any one of 0.5%, 1%, 1.5%, 2%, 2.5%, 3%, 4%, 5%, 6%, 7%, 7.5%, 8%, 9%, 10%, 11%, 12%, 13%, 14%, 15%, 16%, 17%, 18%, 19%, 20%, 25%, or 30% of the PBMCs are NK cells. In some embodiments, at least about 3.5% of the PBMCs are NK cells. In some embodiments, at least about any one of 1%, 2%, 3%, 4%, 5%, 6%, 7%, 8%, 9%, 1, 12%, 14%, 16%, 18%, 20%, 25%, 30%, 35% or 40% of the PBMCs are monocytes. In some embodiments, at least about 4% of the PBMCs are monocytes. In some embodiments, at least about 25% of the PBMCs are T cells; at least about 2.5% of the PBMCs are B cells; at least about 3.5% of the PBMCs are NK cells; and at least about 4% of the PBMCs are monocytes.

In some embodiments according to the methods described herein, not more than about any one of 40%, 45%, 50%, 55%, 60%, 65%, 70%, 75%, 80%, 85%, or 90% of the PBMCs are T cells. In some embodiments, not more than about 70% of the PBMCs are T cells. In some embodiments, not more than about any one of 5%, 10%, 12%, 14%, 16%, 18%, 20%, 22%, 25%, 30%, 35%, 40%, or 50% of the PBMCs are B cells. In some embodiments, not more than about 14% of the PBMCs are B cells. In some embodiments, not more than about any one of 10%, 15%, 20%, 25%, 30%, 35%, 40%, 45%, 50% or 60% of the PBMCs are NK cells. In some embodiments, not more than about 35% of the PBMCs are NK cells. In some embodiments, not more than about any one of 5%, 10, 12%, 14%, 16%, 18%, 20%, 22%, 25%, 30%, 35%, 40%, or 50% of the PBMCs are monocytes. In some embodiments, not more than about 4% of the PBMCs are monocytes. In some embodiments, not more than about 25% of the PBMCs are T cells; not more than about 2.5% of the PBMCs are B cells; not more than about 3.5% of the PBMCs are NK cells; and not more than about 4% of the PBMCs are monocytes.

In some embodiments according to the methods described herein, about any one of 20% to 25%, 25% to 30%, 30% to 35%, 35% to 40%, 40% to 45%, 45% to 50%, 50% to 55%, 55% to 60%, 60% to 65%, 65% to 70%, or 70% to 75% of the modified PBMCs are T cells. In some embodiments, about 25% to about 70% of the modified PBMCs are T cells. In some embodiments, about any one of 1% to 2.5%, 2.5% to 4%, 4% to 6%, 6% to 8%, 8% to 10%, 10% to 12%, 12% to 14%, 14% to 16%, 16% to 20% or 20% to 25% of the modified PBMCs are B cells. In some embodiments, about 2.5% to about 14% of the modified PBMCs are B cells. In some embodiments, about any one of 1% to 2%, 2% to 3.5%, 3.5% to 5%, 5% to 8%, 8% to 10%, 10% to 12%, 12% to 14%, 14% to 16%, 16% to 20% or 20% to 25% of the modified PBMCs are B cells. In some embodiments, about 3.5% to about 35% of the modified PBMCs are NK cells. In some embodiments, about any one of 2% to 4%, 4% to 6%, 6% to 8%, 8% to 10%, 10% to 12%, 12% to 14%, 14% to 16%, 16% to 20%, 20% to 25%, 25% to 30%, 30% to 35%, or 35% to 40% of the modified PBMCs are monocytes. In some embodiments, about 4% to about 25% of the modified PBMCs are monocytes. In some embodiments, about 25% to about 70% of the modified PBMCs are T cells, about 2.5% to about 14% of the modified PBMCs are B cells, about 3.5% to about 35% of the modified PBMCs are NK cells, and about 4% to about 25% of the modified PBMCs are NK cells.

As used herein, PBMCs can also be generated after manipulating the composition of a mixed cell population of mononuclear blood cells (such as lymphocytes and monocytes). In some instances, the PBMCs are generated after reducing (such as depleting) certain subpopulations (such as B cells) within a mixed cell population of mononuclear blood cells. The composition in a mixed cell population of mononuclear blood cells in an individual can be manipulated to make the cell population more closely resemble a leukapheresis product from whole blood in the same individual. In other examples, the composition in a mixed cell population of mononuclear blood cells (for example, mouse splenocytes) can also be manipulated to make the cell population more closely resemble human PBMCs isolated from a leukapheresis product from human whole blood.

In some embodiments of the invention, the composition of nucleated cells comprising an exogenous antigen is a population of cells found in PBMCs. In some embodiments, the composition of nucleated cells comprising an exogenous antigen comprises one or more of T cells, B cells, NK cells, monocytes, dendritic cells or NK-T cells. In some embodiments, the composition of nucleated cells comprising an exogenous antigen comprises one or more of CD3+ T cells, CD20+ B cells, CD14+ monocytes, CD56+NK cells. In some embodiments, the composition of nucleated cells comprising an exogenous antigen comprises at least about any of 70%, 75%, 80%, 85%, 90%, 95%, or 99% T cells. In some embodiments, the composition of nucleated cells comprising an exogenous antigen comprises 100% T cells. In some embodiments, the composition of nucleated cells comprising an exogenous antigen comprises at least about any of 70%, 75%, 80%, 85%, 90%, 95%, or 99% B cells. In some embodiments, the composition of nucleated cells comprising an exogenous antigen comprises 100% B cells. In some embodiments, the composition of nucleated cells comprising an exogenous antigen comprises at least about any of 70%, 75%, 80%, 85%, 90%, 95%, or 99% NK cells. In some embodiments, the composition of nucleated cells comprising an exogenous antigen comprises 100% NK cells. In some embodiments, the composition of nucleated cells comprising an exogenous antigen comprises at least about any of 70%, 75%, 80%, 85%, 90%, 95%, or 99% monocytes. In some embodiments, the composition of nucleated cells comprising an exogenous antigen comprises 100% monocytes. In some embodiments, the composition of nucleated cells comprising an exogenous antigen comprises at least about any of 70%, 75%, 80%, 85%, 90%, 95%, or 99% dendritic cells. In some embodiments, the composition of nucleated cells comprising an exogenous antigen comprises 100% dendritic cells. In some embodiments, the composition of nucleated cells comprising an exogenous antigen comprises at least about any of 70%, 75%, 80%, 85%, 90%, 95%, or 99% NK-T cells. In some embodiments, the composition of nucleated cells comprising an exogenous antigen comprises 100% NK-T cells.

### Antigens

In some embodiments, the invention provides methods for stimulating an immune response in an individual to an exogenous antigen, the method comprising a) administering an effective amount of a composition comprising nucleated cells (e.g., PBMCs) to an individual, wherein the nucleated cells comprise an exogenous antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19). In some embodiments, the exogenous antigen is a disease-associated antigen. In some embodiments, the exogenous antigen is derived from peptides or mRNA isolated from a diseased cell. In some embodiments, the exogenous antigen is a non-self antigen. In some embodiments, the exogenous antigen is a tumor antigen, viral antigen, bacterial antigen, or fungal antigen. In some embodiments, the exogenous antigen is derived from a lysate, such as a lysate of disease cells. In some embodiments, the exogenous antigen is derived from a tumor lysate. In some embodiments, the exogenous antigen is a tumor antigen or a tumor associated antigen. In some embodiments, the exogenous antigen is associated with a cancer. In some embodiments, the cancer is head and neck cancer, cervical cancer, vulvar cancer, vaginal cancer, penile cancer, anal cancer, perianal cancer, anogenital cancer, oral cancer or salivary cancer. In some embodiments, the exogenous antigen is a head and neck cancer antigen, a cervical cancer antigen, a vulvar cancer antigen, a vaginal cancer antigen, a penile cancer antigen, an anal cancer antigen, a perianal cancer antigen, an anogenital cancer antigen, an oral cancer antigen, a salivary cancer antigen, a breast cancer antigen, a skin cancer antigen, a bladder cancer antigen, a colon cancer, a rectal cancer antigen, an endometrial cancer antigen, a kidney cancer antigen, a leukemia antigen, a lung cancer antigen, a melanoma antigen, a non-Hodgkin lymphoma antigen, a pancreatic cancer antigen, a prostate cancer antigen, or a thyroid cancer antigen, In some embodiments, the cancer is a solid cancer. In some embodiments, the cancer is a hematologic cancer. In some embodiments, the cancer is a virus-associated cancer.

In some embodiments, the exogenous antigen is a cancer antigen found in a HPV-associated cancer. In some embodiments, the cancer is a localized cancer. In some embodiments, the cancer is a metastatic cancer. In some embodiments, the exogenous antigen is associated with an infectious disease. In some embodiments, the infectious disease is associated with HIV, HPV, EBV, MCV, HBV or HCV.

In some embodiments according to the methods described herein, the exogenous antigen comprises one or more proteins. In some embodiments, the exogenous antigen is encoded by one or more nucleic acids and enters the nucleated cells in the form of one or more nucleic acids, such as but not limited to DNAs, cDNAs, mRNAs, and plasmids. In some embodiments, the exogenous antigen is encoded by one or more mRNAs and enters the nucleated cells in the form of one or more mRNAs.

In some embodiments according to the methods described herein, the exogenous antigen is a human papillomavirus (HPV) antigen. Papillomaviruses are small nonenveloped DNA viruses with a virion size of ˜55 nm in diameter. More than 100 HPV genotypes are completely characterized, and a higher number is presumed to exist. HPV is a known cause of cervical cancers, as well as some vulvar, vaginal, penile, oropharyngeal, anal, and rectal cancers. Although most HPV infections are asymptomatic and clear spontaneously, persistent infections with one of the oncogenic HPV types can progress to precancer or cancer. Other HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (SILs), cervical intraepithelial neoplasia (CIN), vulvar intraepithelial neoplasia (VIN) and vaginal intraepithelial neoplasia (VAIN). Many of the known human papillomavirus (HPV) types cause benign lesions with a subset being oncogenic. Based on epidemiologic and phylogenetic relationships, HPV types are classified into fifteen “high risk types” (HPV 16, 18, 31, 33, 35, 39, 45, 51, 52, 56, 58, 59, 68, 73, and 82) and three “probable high risk types” (HPV 26, 53, and 66), which together are known to manifest as low and high grade cervical changes and cancers, as well as other anogential cancers such as vulval, vaginal, penile, anal, and perianal cancer, as well as head and neck cancers. Recently, the association of high risk types HPV 16 and 18 with breast cancer was also described. Eleven HPV types classified as “low risk types” (HPV 6, 11, 40, 42, 43, 44, 54, 61, 70, 72, and 81) are known to manifest as benign low-grade cervical changes, genital warts and recurrent respiratory papillomatosis. Cutaneous HPV types 5, 8, and 92 are associated with skin cancer. In some HPV-associated cancers, the immune system is depressed and correspondingly, the antitumor response is significantly impaired. See Suresh and Burtness, Am J Hematol Oncol 13(6):20-27 (2017). In some embodiments, the exogenous antigen is a pool of multiple polypeptides that elicit a response against the same and or different antigens. In some embodiments, an antigen in the pool of multiple antigens does not decrease the immune response directed toward other antigens in the pool of multiple antigens. In some embodiments, the HPV antigen is a polypeptide comprising an antigenic HPV epitope and one or more heterologous peptide sequences. In some embodiments, the HPV antigen complexes with itself, with other antigens, or with the adjuvant. In some embodiments, the HPV is HPV-16 or HPV-18. In some embodiments, the HPV antigen is comprised of an HLA-A2-specific epitope. In some embodiments, the HPV antigen is an HPV E6 antigen or an HPV E7 antigen. In some embodiments, the antigen comprises a peptide derived from HPV E6 and/or E7. In some embodiments, the antigen comprises an HLA-A2-restricted peptide derived from HPV E6 and/or E7. In some embodiments, the HLA-A2-restricted peptide comprises the amino acid sequence of any one of SEQ ID NOs:50-57. In some embodiments, the HPV antigen comprises an amino acid sequence with at least 90% similarity to any one of SEQ ID NOs:50-57. In some embodiments, the HPV antigen comprises an amino acid sequence with at least 90% similarity to SEQ ID NO:50. In some embodiments, the HPV antigen comprises an amino acid sequence with at least 90% similarity to SEQ ID NO:51. In some embodiments, the HPV antigen comprises the amino acid sequence of SEQ ID NO: 52. In some embodiment, the HPV antigen consists of the amino acid sequence of SEQ ID NO: 53. In some embodiments, the HPV antigen comprises the amino acid sequence of SEQ ID NO:54. In some embodiments, the HPV antigen consists of the amino acid sequence of SEQ ID NO:55. In some embodiments, the HPV antigen consists of the amino acid sequence of SEQ ID NO:56. In some embodiments, the HPV antigen consists of the amino acid sequence of SEQ ID NO:57. In some embodiments, the exogenous antigen comprises the amino acid sequence of any one of SEQ ID NOs: x. In some embodiments, the exogenous antigen is a plurality of antigens comprising at least one of the amino acid sequences of any one of SEQ ID NOs:50-57. In some embodiments, the exogenous antigen is a plurality of antigens comprising 2, 3, 4, 5, 6, 7 or 8 of the amino acid sequences of any one of SEQ ID Nos:50-57. In some embodiments, the exogenous antigen is a plurality of antigens comprising an amino acid sequence with at least 90% similarity to SEQ ID NO:51 and an amino acid sequence with at least 90% similarity to SEQ ID NO:55. In a preferred embodiment, the exogenous antigen is a plurality of antigens comprising the amino acid sequence of SEQ ID NO:51 and the amino acid sequence of SEQ ID NO:55. In some embodiments, the plurality of antigens is contained within a pool of non-covalently linked peptides. In some embodiments, the plurality of antigens is contained within a pool of non-covalently linked peptides, wherein each peptide comprises no more than one antigen. In some embodiments, the plurality of antigens is contained within a pool of non-covalently linked peptides, wherein the amino acid sequence of SEQ ID NO: 51 and the amino acid sequence of SEQ ID NO:55 are contained within separate peptides.

In some embodiments according to the methods described herein, the nucleated cells (e.g., PBMCs) comprise a plurality of exogenous antigens that comprise a plurality of immunogenic epitopes. In further embodiments, following administration to an individual of the nucleated cells comprising the plurality of antigens that comprise the plurality of immunogenic epitopes, none of the plurality of immunogenic epitopes decreases an immune response in the individual to any of the other immunogenic epitopes. In some embodiments, the exogenous antigen is a polypeptide and the immunogenic epitope is an immunogenic peptide epitope. In some embodiments, the immunogenic peptide epitope is fused to an N-terminal flanking polypeptide and/or a C-terminal flanking polypeptide. In some embodiments, the exogenous antigen is a polypeptide comprising an immunogenic peptide epitope and one or more heterologous peptide sequences. In some embodiments, the exogenous antigen is a polypeptide comprising an immunogenic peptide epitope that is flanked on the N-terminus and/or the C-terminus by heterologous peptide sequences. In some embodiments, the flanking heterologous peptide sequences are derived from disease-associated immunogenic peptides. In some embodiments, the flanking heterologous peptide sequences are non-naturally occurring sequence. In some embodiments, the flanking heterologous peptide sequences are derived from an immunogenic synthetic long peptide (SLP). In some embodiments, the N-terminal flanking polypeptide comprises the amino acid sequence of any one of SEQ ID NOs: 60-65 and/or the C-terminal flanking polypeptide comprises the amino acid sequence of any one of SEQ ID NOs:66-72. In some embodiments, the exogenous antigen is capable of being processed into an MHC class I-restricted peptide and/or an MHC class II-restricted peptide.

### Adjuvants

As used herein, the term “adjuvant” can refer to a substance which either directly or indirectly modulates and/or engenders an immune response. In some embodiments of the invention, an adjuvant is used to condition a population of nucleated cells such as a population of PBMCs (i.e, the cells are incubated with an adjuvant prior to administration to an individual). In some instances, the adjuvant is administered in conjunction with an exogenous antigen to effect enhancement of an immune response to the exogenous antigen as compared to exogenous antigen alone. Therefore, adjuvants can be used to boost elicitation of an immune cell response (e.g. T cell response) to an exogenous antigen. Exemplary adjuvants include, without limitation, stimulator of interferon genes (STING) agonists, retinoic acid-inducible gene I (RIG-I) agonists, and agonists for TLR3, TLR4, TLR7, TLR8 and/or TLR9. Exemplary adjuvants include, without limitation, CpG ODN, interferon-α (IFN-α), polyinosinic:polycytidylic acid (polyL:C), imiquimod (R837), resiquimod (R848), or lipopolysaccharide (LPS). In some embodiments, the adjuvant is CpG ODN, LPS, IFN-α, STING agonists, RIG-I agonists, poly I:C, R837, R848, a TLR3 agonist, a TLR4 agonist or a TLR 9 agonist. In particular embodiments, the adjuvant is a CpG ODN. In some embodiments, the adjuvant is a CpG ODN. In some embodiments, the CpG ODN is a Class A CpG ODN, a Class B CpG ODN, or a Class C CpG ODN. In some embodiments, the CpG ODN adjuvant comprise of a selection from the group of CpG ODN 1018, CpG ODN 1585, CpG ODN 2216, CpG ODN 2336, CpG ODN 1668, CpG ODN 1826, CPG ODN 2006, CpG ODN 2007, CpG ODN BW006, CpG ODN D-SL01, CpG ODN 2395, CpG ODN M362, CpG ODN D-SL03. In some embodiments, the CpG ODN adjuvant is CpG ODN 1826 (TCCATGACGTTCCTGACGTT (SEQ ID NO:58)) or CpG ODN 2006 (also known as CpG 7909) (TCGTCGTTTTGTCGTTTTGTCGTT (SEQ ID NO:73)) oligonucleotide. In some embodiments, the adjuvant is CpG 7909. In some embodiments, the RIG-I agonist comprises polyinosinic:polycytidylic acid (polyL:C). Multiple adjuvants can also be used in conjunction with exogenous antigens to enhance the elicitation of immune response. In some embodiments, the modified PBMCs comprise more than one adjuvant. Multiple adjuvants can also be used in conjunction with exogenous antigens to enhance the elicitation of immune response. In some embodiments, the modified PBMCs comprise more than one adjuvant. In some embodiments, the modified PBMCs comprise any combination of the adjuvants CpG ODN, LPS, IFN-α, STING agonists, RIG-I agonists, poly I:C, R837, R848, a TLR3 agonist, a TLR4 agonist or a TLR 9 agonist.

### Compositions, Formulations, and Routes of Administration

In some aspects, the invention provides pharmaceutical compositions comprising an immunoconjugate and/or nucleated cells comprising an exogenous antigen as described herein, e.g., for use in any of the below therapeutic methods. In some embodiments, a pharmaceutical composition comprises any of the immunoconjugates provided herein and a pharmaceutically acceptable carrier. In some embodiments, a pharmaceutical composition comprises any of the nucleated cells comprising an exogenous antigen provided herein and a pharmaceutically acceptable carrier.

In some embodiments, the invention provides formulating the immunoconjugate and/or nucleated cells comprising an exogenous antigen with at least one pharmaceutically acceptable carrier, whereby a preparation of immunoconjugate is formulated for administration in vivo.

In some embodiments, pharmaceutical compositions of the present invention comprise a therapeutically effective amount of immunoconjugate dissolved or dispersed in a pharmaceutically acceptable carrier. In some embodiments, pharmaceutical compositions of the present invention comprise a therapeutically effective amount of nucleated cells comprising an exogenous antigen suspended in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains immunoconjugate and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

An immunoconjugate of the invention can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. In some embodiments, the immunoconjugate is administered intratumorally. A composition of nucleated cells comprising an exogenous antigen is typically delivered by intravascular administration.

Parenteral compositions include those designed for administration by injection, e.g. subcutaneous, intradermal, intralesional, intravenous, intraarterial intramuscular, intrathecal or intraperitoneal injection. For injection, the immunoconjugates of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the immunoconjugates may be in powder form for constitution with a suitable vehicle, e.g., sterile pyrogen-free water, before use. Sterile injectable solutions are prepared by incorporating the immunoconjugates of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Pharmaceutical compositions comprising the immunoconjugates of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The immunoconjugates may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

In some embodiments, the nucleated cells comprising an exogenous antigen are formulated in phosphate buffered saline. In some embodiments, the nucleated cells comprising an exogenous antigen are formulated in about 50% (w/w) Cryostor® 10, about 30% HypoThermasol® and about 20% (w/w) of 25% human serum albumin.

### Methods of Treatment

In some aspects, the invention provides methods for stimulating an immune response in an individual, the method comprising a) administering an effective amount of a composition comprising nucleated cells to an individual, wherein the nucleated cells comprise an exogenous antigen; and b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment. In some embodiments, the second polypeptide binds PD-1. In some embodiments, the second polypeptide is an antigen-binding moiety that specifically binds PD-1. In some embodiments, the second polypeptide specifically binds a tumor antigen or an antigen in the tumor cell environment. In some embodiments, the tumor antigen is a Fibroblast activation protein (FAP). In some embodiments, the second polypeptide binds FAP. In some embodiments, the second polypeptide is an antigen-binding moiety that specifically binds FAP. In some embodiments, the exogenous antigen is a disease-associated antigen. In some embodiments, the disease-associated antigen is cancer antigen, an infectious disease antigen, or a viral-associated disease antigen. In some embodiments, the individual has cancer, an infectious disease, or a viral associated disease.

In some embodiments, the invention provides methods for the use of an immunoconjugate of the invention in conjunction with a composition of nucleated cells comprising an exogenous antigen of the invention for stimulating an immune response to a tumor antigen in an individual. In some embodiments, the invention provides methods for the use of an immunoconjugate of the invention in conjunction with a composition of nucleated cells comprising an exogenous antigen of the invention for treating a disease in an individual wherein the disease is cancer, an infectious disease, or a viral-associated disease. In some embodiments, the invention provides methods for the use of an immunoconjugate of the invention in conjunction with a composition of nucleated cells comprising an exogenous antigen of the invention for reducing tumor growth in an individual. In some embodiments, the invention provides methods for the use of an immunoconjugate of the invention in conjunction with a composition of nucleated cells comprising an exogenous antigen of the invention for enhancing the cell-based immunotherapy. In some embodiments, the invention provides methods the use of an immunoconjugate of the invention in conjunction with a composition of nucleated cells comprising an exogenous antigen of the invention for vaccinating an individual in need thereof; for example wherein the individual has a disease responsive to vaccination; wherein the disease is cancer, an infectious disease, or a viral-associated disease.

In some embodiments, the invention provides a composition for use in stimulating an immune response in an individual, wherein the composition comprises an immunoconjugate comprising a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment; wherein the composition is formulated for administration in conjunction with a composition comprising nucleated cells, wherein the nucleated cells comprise an exogenous antigen. In some embodiments, the invention provides a composition for use in stimulating an immune response in an individual, wherein the composition comprises nucleated cells comprising an exogenous antigen; wherein the composition is formulated for administration in conjunction with an immunoconjugate comprising a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment. In some embodiments, the epitope on the T cell is a PD-1 epitope. In some embodiments, the second polypeptide binds PD-1. In some embodiments, the second polypeptide is an antigen-binding moiety that specifically binds PD-1. In some embodiments, the second polypeptide specifically binds a tumor antigen or an antigen in the tumor cell environment. In some embodiments, the tumor antigen is a Fibroblast activation protein (FAP). In some embodiments, the second polypeptide binds FAP. In some embodiments, the second polypeptide is an antigen-binding moiety that specifically binds FAP. In some embodiments, the exogenous antigen is a disease-associated antigen. In some embodiments, the disease-associated antigen is cancer antigen, an infectious disease antigen, or a viral-associated disease antigen. In some embodiments, the individual has cancer, an infectious disease, or a viral associated disease.

In some embodiments, the invention provides the use of an effective amount of an immunoconjugate in the manufacture of a medicament for stimulating an immune response in an individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), wherein the second polypeptide is capable of specific binding to a T cell, a tumor cell, or the tumor cell environment; wherein the immunoconjugate is formulated for administration in conjunction with a composition comprising nucleated cells, wherein the nucleated cells comprise an exogenous antigen. In some embodiments, the invention provides the use of an effective amount of a composition in the manufacture of a medicament for stimulating an immune response in an individual, wherein the composition comprises nucleated cells comprising an exogenous antigen; wherein the composition is formulated for administration in combination with an immunoconjugate comprising a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and the second polypeptide is capable of specific binding to a T cell, a tumor cell, or the tumor cell environment. In some embodiments, the epitope on the T cell is a PD-1 epitope. In some embodiments, the second polypeptide binds PD-1. In some embodiments, the second polypeptide is an antigen-binding moiety that specifically binds PD-1. In some embodiments, the second polypeptide specifically binds a tumor antigen or an antigen in the tumor cell environment. In some embodiments, the tumor antigen is a Fibroblast activation protein (FAP). In some embodiments, the second polypeptide binds FAP. In some embodiments, the second polypeptide is an antigen-binding moiety that specifically binds FAP. In some embodiments, the exogenous antigen is a disease-associated antigen. In some embodiments, the disease-associated antigen is cancer antigen, an infectious disease antigen, or a viral-associated disease antigen. In some embodiments, the individual has cancer, an infectious disease, or a viral associated disease.

In some embodiments, the composition of nucleated cells comprising an exogenous antigen further comprises an adjuvant. In some embodiments, the nucleated cells are conditioned before or after introducing the exogenous antigen into the cells. In some embodiments, the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

In some embodiments, the conditioned nucleated cells are a conditioned plurality of modified PBMCs. In some embodiments, the plurality of modified PBMCs are further modified to increase expression of one or more of co-stimulatory molecules.

In some embodiments of the invention, the immunoconjugate is administered before, at the same time, or after administration of the composition of nucleated cells comprising an exogenous antigen. In some embodiments, the composition of nucleated cells is administered a plurality of times. In some embodiments, the composition of nucleated cells is administered more than one time, two times, three times, four times, five times, six times, seven times, eight times, nine times, ten times, or more than ten times. In some embodiments, the immunoconjugate is administered a plurality of times after administration of the composition of nucleated cells. In some embodiments, the immunoconjugate is administered more than one time, two times, three times, four times, five times, six times, seven times, eight times, nine times, ten times, or more than ten times after administration of the composition of nucleated cells. In some embodiments, the immunoconjugate is administered more than one time, two times, three times, four times, five times, six times, seven times, eight times, nine times, ten times, or more than ten times after each administration of the composition of nucleated cells. In some embodiments, the immunoconjugate is administered to the individual one or more of 1 day, 2 days, 3 days, 3 days, 4 days, 5 days, 6 days, 7 days, 8 days, 9 days, 10 days, 11 days, 12 days, 13 days, 14 days or 21 days after administration of the composition of nucleated cells. In some embodiments, the immunoconjugate is administered to the individual one or more of 0 days, 3 days, 7 days, 14 days or 21 days after administration of the composition of nucleated cells. In some embodiments, the immunoconjugate is administered to the individual on the same day, and 3 days and 7 days after administration of the composition of nucleated cells. In some embodiments, the immunoconjugate is administered to the individual 7 days, 14 days and 21 days after administration of the composition of nucleated cells. In some embodiments, the immunoconjugate is administered to the individual weekly after administration of the composition of nucleated cells. In some embodiments, the immunoconjugate is administered to the individual one or more of 1 day, 2 days, 3 days, 3 days, 4 days, 5 days, 6 days, 7 days, 8 days, 9 days, 10 days, 11 days, 12 days, 13 days, 14 days or 21 days before administration of the composition of nucleated cells. In some embodiments, the immunoconjugate is administered to the individual one or more of 0 days, 3 days, 7 days, 14 days or 21 days before administration of the composition of nucleated cells. In some embodiments, the immunoconjugate is administered to the individual on the same day, and 3 days and 7 days before administration of the composition of nucleated cells. In some embodiments, the immunoconjugate is administered to the individual 7 days, 14 days and 21 days before administration of the composition of nucleated cells. In some embodiments, the immunoconjugate is administered to the individual weekly before administration of the composition of nucleated cells.

In some embodiments, immunoconjugates of the invention for use as a medicament in conjunction with the administration of a composition of nucleated cells comprising an exogenous antigen are provided. In some embodiments, compositions of nucleated cells comprising an exogenous antigen of the invention for use as a medicament in conjuction with the administration of an immunoconjugate are provided. In further aspects, immunoconjugates and compositions of nucleated cells comprising an exogenous antigen of the invention for use in treating a disease in conjunction with one another are provided. In certain embodiments, immunoconjugates and compositions of nucleated cells comprising an exogenous antigen of the invention for use in a method of treatment are provided. In one embodiment, the invention provides immunoconjugates and compositions of nucleated cells comprising an exogenous antigen as described herein for use in the treatment of a disease in an individual in need thereof. In certain embodiments, the invention provides immunoconjugates and compositions of nucleated cells comprising an exogenous antigen for use in a method of treating an individual having a disease comprising administering to the individual a therapeutically effective amount of the immunoconjugate and the composition of nucleated cells comprising an exogenous antigen in conjunction with one another. In certain embodiments, the disease to be treated is a proliferative disorder. In a particular embodiment, the disease is cancer. In certain embodiments, the method further comprises administering to the individual a therapeutically effective amount of at least one additional therapeutic agent, e.g., an anti-cancer agent if the disease to be treated is cancer. In further embodiments, the invention provides immunoconjugates and compositions of nucleated cells comprising an exogenous antigen for use in stimulating the immune system. In certain embodiments, the invention provides immunoconjugates and compositions of nucleated cells comprising an exogenous antigen for use in a method of stimulating the immune system in an individual comprising administering to the individual an effective amount of the immunoconjugate and the composition of nucleated cells comprising an exogenous antigen in conjunction with one another to stimulate the immune system. An “individual” according to any of the above embodiments is a mammal; for example, a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In some embodiments, the invention provides for the use of an immunconjugate of the invention in the manufacture or preparation of a medicament for use in conjunction with a composition of nucleated cells comprising an exogenous antigen. In some embodiments, the invention provides for the use of a composition of nucleated cells comprising an exogenous antigen of the invention in the manufacture or preparation of a medicament for use in conjunction with an immunoconjugate. In one embodiment, the medicament is for the treatment of a disease in an individual in need thereof. In one embodiment, the medicament is for use in a method of treating a disease comprising administering to an individual having the disease a therapeutically effective amount of the medicament. In certain embodiments the disease to be treated is a proliferative disorder. In a particular embodiment, the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in an individual comprising administering to the individual an effective amount of the medicament to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like. In some embodiments, stimulation of the immune system includes tumor infiltration by immune cells such as tumor infilatrating lymphocytes (TILs). In some embodiments, stimulation of the immune system comprises preferential expansion of CD8+ T cells over T_{reg} cells. In some embodiments, the stimulation of the immune system includes eliciting protective immunological memory; for example, to allow a quicker and/or more specific response to subsequent exposure to an antigen.

In some embodiments, the invention provides a method for treating a disease in an individual. In one embodiment, the method comprises administering to an individual having such disease a therapeutically effective amount of an immunoconjugate and a therapeutically effective amount of a composition of nucleated cells comprising an exogenous antigen of the invention. In one embodiment, a composition is administered to said individual, comprising the immunoconjugate of the invention in a pharmaceutically acceptable form in conjunction with administration of a composition of nucleated cells comprising an exogenous antigen of the invention in a pharmaceutically acceptable form. In certain embodiments, the disease to be treated is a proliferative disorder. In a particular embodiment, the disease is cancer. In certain embodiments, the method further comprises administering to the individual a therapeutically effective amount of at least one additional therapeutic agent, e.g., an anti-cancer agent if the disease to be treated is cancer. In a further aspect, the invention provides a method for stimulating the immune system in an individual, comprising administering to the individual an effective amount of an immunoconjugate and an effective amount of a composition of nucleated cells comprising an exogenous antigen to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like. In some embodiments, stimulation of the immune system includes tumor infiltration by immune cells such as tumor infilatrating lymphocytes (TILs). In some embodiments, stimulation of the immune system comprises preferential expansion of CD8+ T cells over T_{reg} cells. In some embodiments, the stimulation of the immune system includes eliciting protective immunological memory; for example, to allow a quicker and/or more specific response to subsequent exposure to an antigen.

In certain embodiments, the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that may be treated using an immunoconjugate of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments, the cancer is chosen from the group consisting of kidney cancer, skin cancer, lung cancer, colorectal cancer, breast cancer, brain cancer, head and neck cancer, prostate cancer and bladder cancer. A skilled artisan readily recognizes that in many cases the immunoconjugates and the composition of nucleated cells comprising an exogenous antigen in conjunction with one another may not provide a cure but may only provide partial benefit. In some embodiments, a physiological change having some benefit is also considered therapeutically beneficial. Thus, in some embodiments, an amount of immunoconjugate and the amount of the composition of nucleated cells comprising an exogenous antigen that provides a physiological change is considered an “effective amount” or a “therapeutically effective amount”. The subject, patient, or individual in need of treatment is typically a mammal, more specifically a human.

### Methods of Producing Immunoconjugates and Compositions of Nucleated Cells Comprising an Exogenous Antigen

In some embodiments, the invention provides methods for producing an immunoconjugate for use in conjunction with a composition comprising nucleated cells for stimulating an immune response in an individual, the method comprising expressing a nucleic acid encoding the immunoconjugate in a cell under conditions to produce the immunoconjugate, wherein the immunoconjugate is a fusion protein comprising a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), wherein the second polypeptide is capable of specific binding to a T cell, a tumor cell, or the tumor cell environment; wherein the immunoconjugate is for use in conjunction with administering a composition comprising nucleated cells, wherein the nucleated cells comprise an exogenous antigen. In other embodiments, the invention provides methods for producing a composition comprising nucleated cells for use in conjunction with an immunoconjugate for stimulating an immune response in an individual, the method comprising introducing an exogenous antigen intracellularly to a population of nucleated cells; wherein the composition is for use in conjunction with administration of an immunoconjugate; wherein the immunoconjugate comprises a mutant IL-2 polypeptide and a second polypeptide, and wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment.

### Methods of Producing Immunoconjugates

Immunoconjugates of the invention may be obtained, for example, by solid-state peptide synthesis (e.g. Merrifield solid phase synthesis) or recombinant production. For recombinant production one or more polynucleotide encoding the immunoconjugate (or a fragment thereof), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of an immunoconjugate (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the immunoconjugate (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the immunoconjugate of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. For example, human IL-2 is translated with a 20 amino acid signal sequence at the N-terminus of the polypeptide, which is subsequently cleaved off to produce the mature, 133 amino acid human IL-2. In certain embodiments, the native signal peptide, e.g. the IL-2 signal peptide or an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the immunoconjugate may be included within or at the ends of the immunoconjugate (fragment) encoding polynucleotide.

In a further embodiment, a host cell comprising one or more polynucleotides of the invention is provided. In certain embodiments, a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) one or more vector comprising one or more polynucleotide that encodes the immunoconjugate of the invention. As used herein, the term “host cell” refers to any kind of cellular system which can be engineered to generate the immunoconjugates of the invention or fragments thereof. Host cells suitable for replicating and for supporting expression of immunoconjugates are well known in the art. Such cells may be transfected or transduced as appropriate with the particular expression vector and large quantities of vector containing cells can be grown for seeding large scale fermenters to obtain sufficient quantities of the immunoconjugate for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr− CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a mutant-IL-2 polypeptide fused to either the heavy or the light chain of an antibody may be engineered so as to also express the other of the antibody chains such that the expressed mutant IL-2 fusion product is an antibody that has both a heavy and a light chain.

In one embodiment, a method of producing an immunoconjugate according to the invention is provided, wherein the method comprises culturing a host cell comprising one or more polynucleotide encoding the immunoconjugate, as provided herein, under conditions suitable for expression of the immunoconjugate, and optionally recovering the immunoconjugate from the host cell (or host cell culture medium).

Further chemical modification of the immunoconjugate of the invention may be desirable. For example, problems of immunogenicity and short half-life may be improved by conjugation to substantially straight chain polymers such as polyethylene glycol (PEG) or polypropylene glycol (PPG) (see e.g. WO 87/00056).

Immunoconjugates prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the immunoconjugate binds. For example, an antibody which specifically binds the mutant IL-2 polypeptide may be used. For affinity chromatography purification of immunoconjugates of the invention, a matrix with protein A or protein G may be used. For example, sequential Protein A or G affinity chromatography and size exclusion chromatography can be used to isolate an immunoconjugate essentially as described in the Examples. The purity of the immunoconjugate can be determined by any of a variety of well known analytical methods including gel electrophoresis, high pressure liquid chromatography, and the like.

### Methods of Producing Compositions of Nucleated Cells Comprising an Exogenous Antigen

In some embodiments, the invention provides compositions of nucleated cells comprising an exogenous antigen for use in conjunction with an immunoconjugate of the invention for stimulating an immune response. In some embodiments, the nucleated cells are immune cells; for example, a plurality of PBMCs or one or more of T cell, B cell, NK cell, monocytes, dendritic cells or NK-T cells. In some embodiments, the exogenous antigen is delivered to the nucleated cells intracellularly. Methods of introducing exogenous antigens to nucleated cells are known in the art.

In some embodiments, exogenous antigens are introduced into the nucleated cells by passing the cell through a constriction such that transient pores are introduced to the membrane of the cell thereby allowing the exogenous antigen to enter the cell. Examples of constriction-based delivery of compounds into a cell are provided by WO 2013/059343, WO 2015/023982, WO 2016/070136, WO2017041050, WO2017008063, WO 2017/192785, WO 2017/192786, WO 2019/178005, WO 2019/178006, WO 2020/072833, PCT/US2020/15098, and PCT/US2020/020194.

In some embodiments, the exogenous antigen is delivered into the nucleated cells to produce the nucleated cells of the invention by passing a cell suspension comprising the nucleated cells (e.g., PBMCs) through a constriction, wherein the constriction deforms the cells thereby causing a perturbation of the cells such that an exogenous antigen enters the cells. In some embodiments, the constriction is contained within a microfluidic channel. In some embodiments, multiple constrictions can be placed in parallel and/or in series within the microfluidic channel.

In some embodiments, the constriction within the microfluidic channel includes an entrance portion, a centerpoint, and an exit portion. In some embodiments, the length, depth, and width of the constriction within the microfluidic channel can vary. In some embodiments, the width of the constriction within the microfluidic channel is a function of the diameter of the nucleated cells. Methods to determine the diameter of nucleated cells are known in the art; for example, high-content imaging, cell counters or flow cytometry.

In some embodiments of the constriction-based delivery of an exogenous antigen to nucleated cells, the width of the constriction is about 3 μm to about 15 μm. In some embodiments, the width of the constriction is about 3 μm to about 10 μm. In some embodiments, the width of the constriction is about 3 μm to about 6 μm. In some embodiments, the width of the constriction is about 4.2 μm to about 6 μm. In some embodiments, the width of the constriction is about 4.2 μm to about 4.8 μm. In some embodiments, the width of the constriction is about or less than any one of 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm, 11.5 μm, 12 μm, 12.5 μm, 13 μm, 13.5 μm, 14 μm, 14.5 μm or 15 μm. In some embodiments, the width of the constriction is about or less than any one of 4.0 μm, 4.1 μm, 4.2 μm, 4.3 μm, 4.4 μm, 4.5 μm, 4.6 μm, 4.7 μm, 4.8 μm, 4.9 μm, or 5.0 μm. In some embodiments, the width of the constriction is about 4.5 μm.

In some embodiments of the invention, the composition comprises a plurality of nucleated cells (e.g., a plurality of PBMCs) within the population of nucleated cells. In some embodiments, the width of the constriction is about 10% to about 99% of the mean diameter of a subpopulation of nucleated cells having the smallest diameter within the population of nucleated cells. In some embodiments, the width of the constriction is any one of about 10% to about 90%, about 10% to about 80%, about 10% to about 70%, about 20% to about 60%, about 40% to about 60%, about 30% to about 45%, about 50% to about 99%, about 50% to about 90%, about 50% to about 80%, about 50% to about 70%, about 60% to about 90%, about 60% to about 80%, or about 60% to about 70% of the mean diameter of a subpopulation of nucleated cells having the smallest diameter within the population of nucleated cells. In some embodiments, the width of the constriction is any one of about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of the mean diameter of a subpopulation of nucleated cells having the smallest diameter within the population of nucleated cells. In some embodiments, the width of the constriction is any one of about 10%, 15%, 20%, 25%, 30%, 35%, 40%, 45%, 50%, 55%, 60%, 65%, 70%, 75%, 80%, 85%, 90%, 95%, or 99% of the mean diameter of a subpopulation of nucleated cells having the smallest diameter within the population of nucleated cells. In some embodiments, the subpopulation of nucleated cells having the smallest mean diameter within a plurality of input PBMCs is a population of lymphocytes, wherein the diameter of the population of lymphocytes is about 6 μm to about 10 μm. In some embodiments, the mean diameter of the population of lymphocytes is about 7 μm. In some embodiments, the population of lymphocytes is a population of T cells. In some embodiments, the lymphocytes are T cells. In some embodiments, the subpopulation of nucleated cells having the smallest mean diameter within the plurality of input PBMCs are T cells.

In some embodiments of the invention, the composition comprises a plurality of nucleated cells (e.g., a plurality of PBMCs) within the population of nucleated cells. In some embodiments, the width of the constriction is about 10% to about 99% of the mean diameter of a subpopulation of nucleated cells having the largest diameter within the population of nucleated cells. In some embodiments, the width of the constriction is any one of about 10% to about 90%, about 10% to about 80%, about 10% to about 70%, about 20% to about 60%, about 40% to about 60%, about 30% to about 45%, about 15% to about 30%, about 15% to about 20%, about 20% to about 25%, about 25% to about 30%, about 20% to about 30%, about 30% to about 70%, or about 30% to about 60% of the mean diameter of a subpopulation of nucleated cells having the largest diameter within the population of nucleated cells. In some embodiments, the width of the constriction is any one of about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 99% of the mean diameter of a subpopulation of nucleated cells having the largest diameter within the population of nucleated cells. In some embodiments, the width of the constriction is any one of about 5%, 10%, 15%, 20%, 25%, 30%, 35%, 40%, 45%, 50%, 55%, 60%, 65%, 70%, 75%, 80%, 85%, 90%, 95%, or 99% of the mean diameter of a subpopulation of nucleated cells having the largest diameter within the population of nucleated cells. In some embodiments, the subpopulation of nucleated cells having the largest mean diameter within a plurality of input PBMCs is a population of monocytes, wherein the diameter of the population of monocytes is about 15 μm to about 25 μm. In some embodiments, the mean diameter of the population of monocytes is about 18 μm. In some embodiments, the subpopulation of nucleated cells having the largest mean diameter within the plurality of input PBMCs are monocytes.

A number of parameters may influence the delivery of a compound to nucleated cells for stimulating an immune response by the methods described herein. In some embodiments, the cell suspension is contacted with the compound before, concurrently, or after passing through the constriction. The nucleated cells may pass through the constriction suspended in a solution that includes the compound to deliver, although the compound can be added to the cell suspension after the nucleated cells pass through the constriction. In some embodiments, the compound to be delivered is coated on the constriction.

Examples of parameters that may influence the delivery of the compound into the nucleated cells include, but are not limited to, the dimensions of the constriction, the entrance angle of the constriction, the surface properties of the constrictions (e.g., roughness, chemical modification, hydrophilic, hydrophobic, etc.), the operating flow speeds (e.g., cell transit time through the constriction), the cell concentration, the concentration of the compound in the cell suspension, buffer in the cell suspension, and the amount of time that the nucleated cells recover or incubate after passing through the constrictions can affect the passage of the delivered compound into the nucleated cells. Additional parameters influencing the delivery of the compound into the nucleated cells can include the velocity of the nucleated cells in the constriction, the shear rate in the constriction, the viscosity of the cell suspension, the velocity component that is perpendicular to flow velocity, and time in the constriction. In addition, multiple chips comprising channels in series and/or in parallel may impact delivery to nucleated cells. Multiple chips in parallel may be useful to enhance throughput. Such parameters can be designed to control delivery of the compound. In some embodiments, the cell concentration ranges from about 10 to at least about 10¹² cells/mL or any concentration or range of concentrations therebetween. In some embodiments, delivery compound concentrations can range from about 10 ng/mL to about 1 g/mL or any concentration or range of concentrations therebetween. In some embodiments, delivery compound concentrations can range from about 1 pM to at least about 2 M or any concentration or range of concentrations therebetween.

In some embodiments, the concentration of exogenous antigen incubated with the nucleated cells is between about 0.01 μM and about 10 mM. For example, in some embodiments, the concentration of exogenous antigen incubated with the nucleated cells is any of less than about 0.01 μM, about 0.1 μM, about 1 μM, about 10 μM, about 100 μM, about 1 mM or about 10 mM. In some embodiments, the concentration of exogenous antigen incubated with the nucleated cells is greater than about 10 mM. In some embodiments, the concentration of exogenous antigen incubated with the nucleated cells is any of between about 0.01 μM and about 0.1 μM, between about 0.1 μM and about 1 μM, between about 1 μM and about 10 μM, between about 10 μM and about 100 μM, between about 100 μM and about 1 mM, or between 1 mM and about 10 mM. In some embodiments, the concentration of exogenous antigen incubated with the nucleated cells is between about 0.1 μM and about 1 mM. In some embodiments, the concentration of exogenous antigen incubated with the nucleated cells is between about 0.1 μM and about 10 μM. In some embodiments, the concentration of exogenous antigen incubated with the nucleated cells is 1 μM.

In some embodiments, the nucleated cells comprise the nucleic acid encoding the exogenous antigen at a concentration between about 1 nM and about 1 mM. In some embodiments, the nucleated cells comprises the nucleic acid encoding the exogenous antigen at a concentration of any of less than about 0.1 nM, about 1 nM, about 0.01 μM, about 0.1 μM, about 1 μM, about 10 μM, about 100 μM, about 1 mM or about 10 mM. In some embodiments, the nucleated cells comprise the nucleic acid encoding the exogenous antigen at a concentration of greater than about 10 mM. In some embodiments, the nucleated cells comprise the nucleic acid encoding the exogenous antigen at a concentration of any of between about 0.1 nM to about 1 nM, about 1 nM to about 10 nM, about 10 nM to about 100 nM, about 0.1 μM and about 1 μM, between about 1 μM and about 10 μM, between about 10 μM and about 100 μM, between about 100 μM and about 1 mM, or between 1 mM and about 10 mM. In some embodiments, the nucleated cells comprise the nucleic acid encoding the exogenous antigen at a concentration between about 10 nM and about 100 nM. In some embodiments, the nucleated cells comprise the nucleic acid encoding the exogenous antigen at a concentration between about 1 nM and about 10 nM. In some embodiments, the nucleated cells comprise the exogenous antigen at a concentration of about 50 nM. In some embodiments, the nucleic acid is an mRNA.

### Conditioning of Cells

In some embodiments according to any one of methods described herein; the nucleated cells (e.g., PBMCs) comprising an exogenous antigen are conditioned. In further embodiments, the nucleated cells are matured. In some embodiments, the nucleated cells are conditioned subsequent to constriction mediated delivery. In some embodiments, the nucleated cells comprising the exogenous antigen is incubated with an adjuvant for a sufficient time for the cells comprising the constriction-delivered exogenous antigen to condition, thereby generating a composition of conditioned cells comprising the exogenous antigen. In some embodiments, the nucleated cells are conditioned subsequent to constriction-mediated delivery. In some embodiments, the nucleated cells comprising the constriction-delivered exogenous antigen are incubated with an adjuvant for a sufficient time for the nucleated cells comprising the constriction-delivered exogenous antigen to condition, thereby generating a composition of conditioned nucleated cells comprising the exogenous antigen. In some aspects, there is provided a composition of conditioned nucleated cells comprising an exogenous antigen, prepared by a process comprising the steps of: a) passing a cell suspension through a cell-deforming constriction, wherein a width of the constriction is a function of the nucleated cells in the suspension, thereby causing perturbations of the nucleated cells large enough for the exogenous antigen to pass through to form perturbed nucleated cells; b) incubating the perturbed nucleated cells with the exogenous antigen for a sufficient time to allow the exogenous antigen to enter the perturbed nucleated cells, thereby generating modified nucleated cells comprising the exogenous antigen; and c) incubating the modified nucleated cells comprising the constriction-delivered exogenous antigen with an adjuvant for a sufficient time for the modified nucleated cells comprising the constriction-delivered exogenous antigen to condition, thereby generating the composition of conditioned nucleated cells comprising the exogenous antigen. In some embodiments, the process further comprises isolating the modified nucleated cells comprising the exogenous antigen from the cell suspension before incubation with the adjuvant to condition the modified nucleated cells.

In some embodiments, the nucleated cells (e.g., PBMCs) are conditioned prior to constriction-mediated delivery. In some embodiments, the nucleated cells are incubated with an adjuvant for a sufficient time for the nucleated cells to condition, thereby conditioned nucleated cells. In some embodiments, there is provided a composition of conditioned nucleated cells comprising an exogenous antigen, prepared by a process comprising the steps of: a) incubating nucleated cells with an adjuvant for a sufficient time for the nucleated cells to condition, thereby generating conditioned nucleated cells; b) passing a cell suspension comprising the conditioned nucleated cells through a cell-deforming constriction, wherein a width of the constriction is a function of a diameter of the nucleated cells in the suspension, thereby causing perturbations of the nucleated cells large enough for the exogenous antigen to pass through to form conditioned perturbed nucleated cells; and c) incubating the conditioned perturbed nucleated cells with the exogenous antigen for a sufficient time to allow the exogenous antigen to enter the conditioned perturbed nucleated cells, thereby generating the conditioned nucleated cells comprising the exogenous antigen. In some embodiments, the process further comprises isolating the conditioned nucleated cells from the adjuvant before passing the conditioned nucleated cells through a cell-deforming constriction.

In some embodiments according to any one of methods described herein, the nucleated cells (e.g., PBMCs) comprising the exogenous antigen are incubated with the adjuvant for about 1 to about 24 hours for the nucleated cells to condition. In some embodiments, the nucleated cells are incubated with the adjuvant for about 2 to about 10 hours for the nucleated cells to condition. In some embodiments, the nucleated cells are incubated with the adjuvant for about 3 to about 6 hours for the nucleated cells to condition. In some embodiments, the nucleated cells are incubated with the adjuvant for any one of about 1 hour, 2 hours, 3 hours, 3.5 hours, 4 hours, 4.5 hours, 5 hours, 5.5 hours, 6 hours, 8 hours, 12 hours, 16 hours, 20 hours, or 24 hours for the nucleated cells to condition. In some embodiments, the nucleated cells are incubated with the adjuvant for about 4 hours for the nucleated cells to condition.

In some embodiments, there is provided a conditioned plurality of PBMCs comprising an exogenous antigen, prepared by incubating the plurality of PBMCs comprising the exogenous antigen with an adjuvant for a sufficient time for the PBMCs to condition, thereby generating the conditioned plurality of PBMCs comprising the exogenous antigen. In some embodiments, there is provided a conditioned plurality of PBMCs comprising an exogenous antigen, prepared by incubating the plurality of PBMCs with an adjuvant for a sufficient time for the PBMCs to condition prior to introducing the exogenous antigen to the PBMCs, thereby generating the conditioned plurality of PBMCs comprising the exogenous antigen.

In some embodiments according to any of the conditioned plurality of PBMCs described herein, the plurality of PBMCs is incubated with the adjuvant for about 1 to about 24 hours for the PBMCs to condition. In some embodiments, the plurality of PBMCs is incubated with the adjuvant for about 2 to about 10 hours for the PBMCs to condition. In some embodiments, the plurality of PBMCs is incubated with the adjuvant for about 3 to about 6 hours for the PBMCs to condition. In some embodiments, the plurality of PBMCs is incubated with the adjuvant for any one of about 1 hour, 2 hours, 3 hours, 3.5 hours, 4 hours, 4.5 hours, 5 hours, 5.5 hours, 6 hours, 8 hours, 12 hours, 16 hours, 20 hours, or 24 hours for the PBMCs to condition. In some embodiments, the plurality of PBMCs is incubated with the adjuvant for about 4 hours for the PBMCs to condition.

In some embodiments according to any one of the conditioned plurality of PBMCs described herein, one or more co-stimulatory molecules are upregulated in the conditioned plurality of modified PBMCs compared to an unconditioned plurality of modified PBMCs. In some embodiments, one or more co-stimulatory molecules are upregulated in a subpopulation of cells in the conditioned plurality of modified PBMCs compared to the subpopulation of cells in an unconditioned plurality of modified PBMCs. In some embodiments, one or more co-stimulatory molecules are upregulated in the B cells of the conditioned plurality of modified PBMCs compared to the B cells in an unconditioned plurality of modified PBMCs. In some embodiments, the co-stimulatory molecule is CD80 and/or CD86. In some embodiments, the co-stimulatory molecule is CD86. In some embodiments, the CD80 and/or CD86 is upregulated in the B cells of the conditioned plurality of modified PBMCs by more than about 1.2-fold, 1.5-fold, 1.8-fold, 2-fold, 3-fold, 4-fold, 5-fold, 8-fold, or more than 10-fold compared to the B cells in an unconditioned plurality of modified PBMCs. In some embodiments, the CD80 and/or CD86 is upregulated in the B cells of the conditioned plurality of modified PBMCs by any of about 1.2-fold to about 1.5-fold, about 1.5-fold to about 1.8-fold, about 1.8-fold to about 2-fold, about 2-fold to about 3-fold, about 3-fold to about 4-fold, about 4-fold to about 5-fold, about 5-fold to about 8-fold, about 8-fold to about 10-fold, about 10-fold to about 20-fold, about 20-fold to about 50-fold, about 50-fold to about 100-fold, about 100-fold to about 200-fold, about 200-fold to about 500-fold, or more than about 500-fold compared to the B cells in an unconditioned plurality of modified PBMCs. In some embodiments, the expression of one or more of IFN-γ, IL-6, MCP-1, MIP-1β, IP-10, or TNF-α is increased in the conditioned plurality of modified PBMCs compared to an unconditioned plurality of modified PBMCs. In some embodiments, the expression of one or more of IFN-γ, IL-6, MCP-1, MIP-1β, IP-10, or TNF-α is increased a subpopulation of cells in the conditioned plurality compared to the subpopulation of cells in an unconditioned plurality of modified PBMCs. In some embodiments, the expression of one or more of IFN-γ, IL-6, MCP-1, MIP-1β, IP-10, or TNF-α is increased by about 1.2-fold, 1.5-fold, 1.8-fold, 2-fold, 3-fold, 4-fold, 5-fold, 8-fold, or more than 10-fold in the conditioned plurality of modified PBMCs compared to an unconditioned plurality of modified PBMCs. In some embodiments, the expression of one or more of IFN-γ, IL-6, MCP-1, MIP-1β, IP-10, or TNF-α is increased by any of about 1.2-fold to about 1.5-fold, about 1.5-fold to about 1.8-fold, about 1.8-fold to about 2-fold, about 2-fold to about 3-fold, about 3-fold to about 4-fold, about 4-fold to about 5-fold, about 5-fold to about 8-fold, about 8-fold to about 10-fold, about 10-fold to about 20-fold, about 20-fold to about 50-fold, about 50-fold to about 100-fold, about 100-fold to about 200-fold, about 200-fold to about 500-fold, or more than about 500-fold in the conditioned plurality of modified PBMCs compared to an unconditioned plurality of modified PBMCs.

### Further Modifications of the Composition of Nucleated Cells Comprising an Exogenous Antigen

In some embodiments according to any one of the methods described herein, the composition of nucleated cells (e.g., PBMCs) further comprises an agent that enhances the viability and/or function of the nucleated cells as compared to a corresponding composition of nucleated cells that does not comprise the agent. In some embodiments, the composition of nucleated cells further comprises an agent that enhances the viability and/or function of the nucleated cells upon freeze-thaw cycle as compared to a corresponding composition of nucleated cells that does not comprise the agent. In some embodiments, the agent is a cyropreservation agent and/or a hypothermic preservation agent. In some embodiments, the cyropreservation agent nor the hypothermic preservation agent cause not more than 10% or 20% of cell death in a composition of nucleated cells comprising the agent compared to a corresponding composition of nucleated cells that does not comprise the agent before any freeze-thaw cycles. In some embodiments, at least about 70%, about 80%, or about 90% of the nucleated cells are viable after up to 1, 2, 3, 4, 5 freeze-thaw cycles. In some embodiments, the agent is a compound that enhances endocytosis, a stabilizing agent or a co-factor. In some embodiments, the agent is albumin. In some embodiments, the albumin is mouse, bovine, or human albumin. In some embodiments, the agent is human albumin. In some embodiments, the agent is one or more of: a divalent metal cation, glucose, ATP, potassium, glycerol, trehalose, D-sucrose, PEG1500, L-arginine, L-glutamine, or EDTA. In some embodiments, the divalent metal cation is one more of Mg2+, Zn2+ or Ca2+. In some embodiments, the agent is one or more of: sodium pyruvate, adenine, trehalose, dextrose, mannose, sucrose, human serum albumin (HSA), DMSO, HEPES, glycerol, glutathione, inosine, dibasic sodium phosphate, monobasic sodium phosphate, sodium metal ions, potassium metal ions, magnesium metal ions, chloride, acetate, gluoconate, sucrose, potassium hydroxide, or sodium hydroxide. In some embodiments, the agent is one or more of: Sodium pyruvate, adenine, Rejuvesol®, trehalose, dextrose, mannose, sucrose, human serum albumin (HSA), PlasmaLyte®, DMSO, Cryostor® CS2, Cryostor® CS5, Cryostor® CS10, Cryostor® CS15, HEPES, glycerol, glutathione, HypoThermosol®.

In some embodiments according to any one of the methods described herein, the composition of nucleated cells comprises a plurality of modified PBMCs that are further modified to increase expression of one or more of co-stimulatory molecules. In some embodiments, the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TTM4, SLAM, CD48, CD58, CD155, or CD112. In some embodiments, the plurality of modified PBMCs comprises a nucleic acid that results in increased expression of the one or more co-stimulatory molecules. In some embodiments, the plurality of modified PBMCs comprises an mRNA that results in increased expression of the one or more co-stimulatory molecules. In some embodiments, the co-stimulatory molecule is a Signal 2 effector in stimulating T cell activation.

In some embodiments according to any one of the methods described herein, the modified PBMCs are further modified to increase expression of one or more cytokines. In some embodiments, the cytokine is one or more of IL-2, IL-12, IL-21, or IFNα2. In some embodiments, the plurality of modified PBMCs comprises a nucleic acid that results in increased expression and/or secretion of the one or more cytokines. In some embodiments, the cytokine is a Signal 3 effector in stimulating T cell activation.

In some embodiments according to any one of the methods described herein, at least one cell in the plurality of modified PBMCs is positive for expression of HLA-A2. In some embodiments, the modified PBMCs comprise a further modification to modulate MHC class I expression. In some embodiments, the modified PBMCs comprise a further modification to modulate expression of HLA-A02 MHC class I. In some embodiments, the modified PBMCs comprise a further modification to modulate MHC class II expression. In some embodiments, an innate immune response mounted in an individual in response to administration, in an allogeneic context, of the modified PBMCs is reduced compared to an innate immune response mounted in an individual in response to administration, in an allogeneic context, of corresponding modified PBMCs that do not comprise the further modification. In some embodiments, the circulating half-life of the modified PBMCs in an individual to which they were administered is increased compared to the circulating half-life of corresponding modified PBMCs that do not comprise the further modification in an individual to which they were administered. In some embodiments, the circulating half-life of the modified PBMCs in an individual to which they were administered is increased by about any one of 10%, 25%, 50%, 75%, 100%, 2-fold, 3-fold, 4-fold, 5-fold, 10-fold, 25-fold, 50-fold, 100-fold, 200-fold, or 500-fold or more compared to the circulating half-life of corresponding modified PBMCs that do not comprise the further modification in an individual to which they were administered. In some embodiments, the circulating half-life of the modified PBMCs in an individual to which they were administered is essentially the same as the circulating half-life of corresponding modified PBMCs that do not comprise the further modification in an individual to which they were administered.

In some embodiments according to any one of the methods described herein, the process further comprises a step of incubating the composition of nucleated cells with an agent that enhances the viability and/or function of the nucleated cells compared to corresponding nucleated cells prepared without the further incubation step.

### Systems and Kits

In some aspects, the invention provides kits or articles of manufacture for use in modulating an immune response in an individual. In some embodiments, the kit comprises a composition of nucleated cells comprising an exogenous antigen, and an immunoconjugate, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment; wherein the composition and the immunoconjugate are for use in combination for stimulating an immune response to the exogenous antigen in an individual. In some embodiments, the kit comprises a composition of nucleated cells comprising an exogenous antigen, wherein the composition is for use in conjunction with an immunoconjugate for stimulating an immune response to the exogenous antigen in an individual, and wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment. In some embodiments, the kit comprises an immunoconjugate, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment; wherein the immunoconjugate is for use in conjunction with a composition of cells comprising an exogenous antigen for stimulating an immune response to the exogenous antigen in an individual. In some embodiments, the kits comprise components described herein (e.g. a composition of nucleated cells comprising an exogenous antigen and/or an immunoconjugate) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The invention also provides kits comprising components of the methods described herein and may further comprise instructions for performing said methods to modulate an immune response in an individual and/or instructions for introducing an exogenous antigen into nucleated cells. The kits described herein may further include other materials, including buffers, diluents, filters, needles, syringes, and package inserts with instructions for performing any of the methods described herein; e.g., instructions for modulating an immune response in an individual or instructions for modifying nucleated cells to contain an exogenous antigen.

### EXEMPLARY EMBODIMENTS

The invention provides the following enumerated embodiments.

1. A method for stimulating an immune response in an individual, the method comprising

a) administering an effective amount of a composition comprising nucleated cells to an individual, wherein the nucleated cells comprise an exogenous antigen; and

b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

2. A method for stimulating an immune response to a tumor antigen in an individual, the method comprising

a) administering an effective amount of a composition comprising nucleated cells to an individual, wherein the nucleated cells comprise an exogenous tumor antigen; and

b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

3. A method for enhancing a nucleated cell-based immunotherapy, the method comprising administering an effective amount of an immunoconjugate in conjunction with the nucleated cell-based immunotherapy, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO: 19), and wherein the second polypeptide is capable of specific binding to a T cell, a tumor cell, or the tumor cell environment.

4. A method for treating a disease in an individual, the method comprising

a) administering an effective amount of a composition comprising nucleated cells to an individual, wherein the nucleated cells comprise an exogenous antigen associated with the disease; and

b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

5. A method of vaccinating an individual in need thereof, the method comprising

a) administering an effective amount of a composition comprising nucleated cells to an individual, wherein the nucleated cells comprise an exogenous antigen; and

b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

6. The method of embodiment 5, wherein the individual has a disease responsive to vaccination.

7. The method of any one of embodiments 4-6, wherein the disease is cancer, an infectious disease, or a viral-associated disease.

8. A method for reducing tumor growth in an individual, the method comprising

a) administering an effective amount of a composition comprising nucleated cells to an individual, wherein the nucleated cells comprise an exogenous tumor antigen; and

b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

9. The method of any one of embodiments 1-8, wherein the second polypeptide binds a T cell.

10. The method of embodiment 11, wherein the second polypeptide binds PD-1 expressed on the T cell.

11. The method of embodiment 10, wherein the second polypeptide is an antigen-binding moiety that specifically binds PD-1.

12. The method of embodiment 11, wherein the anti-PD-1 antigen-binding moiety comprises

(a) a heavy chain variable region (VH) comprising a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6; or

(b) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO:13.

13. The method of embodiment 11 or 12, wherein the anti-PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO: 14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO:17, and SEQ ID NO:18.

14. The method of any one of embodiments 11-13, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO:15, SEQ ID NO:16, SEQ ID NO: 17, and SEQ ID NO:18.

15. The method of any one of embodiments 11-14, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO: 15.

16. The method of any one of embodiments 11-15, wherein the immunoconjugate comprises a polypeptide comprising an amino acid sequence that is at least about 80%, 85%, 90%, 95%, 96%, 97%, 98%, 99% or 100% identical to the sequence of SEQ ID NO:22, a polypeptide comprising an amino acid sequence that is at least about 80%, 85%, 90%, 95%, 96%, 97%, 98%, 99% or 100% identical to the sequence of SEQ ID NO:24, and a polypeptide comprising an amino acid sequence that is at least about 80%, 85%, 90%, 95%, 96%, 97%, 98%, 99% or 100% identical to the sequence of SEQ ID NO:25.

17. The method of any one of embodiments 11-16, wherein the immunoconjugate comprises a polypeptide sequence of SEQ ID NO:22, a polypeptide sequence of SEQ ID NO:24, and two polypeptide sequences of SEQ ID NO:25.

18. The method of any one of embodiments 1-8, wherein the second polypeptide specifically binds a target antigen presented on a tumor cell or in a tumor cell environment.

19. The method of embodiment 18, wherein the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP).

20. The method of any one of embodiments 1-8 and 18-19, wherein the second polypeptide binds FAP.

21. The method of embodiment 19 of 20, wherein the second polypeptide is an antigen-binding moiety that specifically binds FAP.

22. The method of embodiment 21, wherein the antigen-binding moiety the specifically bind FAP comprising (i) the heavy chain variable region sequence of SEQ ID NO:29 and the light chain variable region sequence of SEQ ID NO: 28; (ii) the heavy chain variable region sequence of SEQ ID NO:31 and the light chain variable region sequence of SEQ ID NO:30; (iii) the heavy chain variable region sequence of SEQ ID NO: 33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36.

23. The method of embodiment 21 or 22, wherein the antigen-binding moiety the specifically bind FAP comprising (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO: 231; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41.

24. The method of any one of embodiments 21-23, wherein antigen-binding moiety the specifically bind FAP comprises the heavy chain variable region sequence of SEQ ID NO:33 and the light chain variable region sequence of SEQ ID NO:32.

25. The method of any one of embodiments 21-24, wherein the immunoconjugate comprises a polypeptide sequence of SEQ ID NO:38, a polypeptide sequence of SEQ ID NO:39, and a polypeptide sequence of SEQ ID NO:40.

26. The method of any one of embodiments 1-25, wherein the mutant IL-2 polypeptide further comprises the amino acid substitution T3A and/or the amino acid substitution C125A.

27. The method of any one of embodiments 1-26, wherein the mutant IL-2 polypeptide comprises an amino acid sequence that is at least about 95%, 96%, 97%, 98%, 99% or 100% identical to the amino acid sequence of SEQ ID NO:20, wherein the mutant IL-2 polypeptide shows reduced affinity to the high-affinity IL-2 receptor and substantially similar affinity to the intermediate-affinity IL-2 receptor, each compared to a wild-type IL-2 polypeptide.

28. The immunoconjugate of any one of embodiments 1-27, wherein the mutant IL-2 polypeptide comprises the amino acid sequence of SEQ ID NO:20.

29. The method of any one of embodiments 1-28, wherein the nucleated cells are immune cells.

30. The method of any one of embodiments 1-29, wherein the nucleated cells are a plurality of peripheral blood mononuclear cells (PBMCs).

31 The method of embodiment 30, wherein the plurality of PBMCs comprise two or more of T cell, B cell, NK cell, monocytes, dendritic cells or NK-T cells.

32. The method of any one of embodiments 1-31, wherein the nucleated cells are one or more of T cells, B cells, NK cells, monocytes, dendritic cells and/or NK-T cells.

33. The method of any one of embodiments 1-32, wherein the exogenous antigen is delivered to the nucleated cells intracellularly.

34. The method of any one of embodiments 1-33, wherein the exogenous antigen is a disease-associated antigen.

35. The method of embodiment 34, wherein the disease-associated antigen is a cancer antigen, an infectious disease antigen, or a viral-associated disease antigen.

36. The method of any one of embodiments 1-35, wherein the exogenous antigen is a human papillomavirus (HPV) antigen.

37. The method of any one of embodiments 1-36, wherein the composition further comprises an adjuvant.

38. The method of embodiment 37, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

39. The method of any one of embodiments 1-38, wherein the nucleated cells comprising the exogenous antigen are prepared by a process comprising the steps of:

a) passing a cell suspension comprising input nucleated cells through a cell-deforming constriction, wherein a diameter of the constriction is a function of a diameter of the input nucleated cells in the suspension, thereby causing perturbations of the input nucleated cells large enough for the exogenous antigen to pass through to form perturbed input nucleated cells;

b) incubating the perturbed input nucleated cells with the exogenous antigen for a sufficient time to allow the exogenous antigen to enter the perturbed input nucleated cells, thereby generating nucleated cells comprising the exogenous antigen.

40. The method of embodiment 39, wherein the width of the constriction is about 10% to about 99% of the mean diameter of the input nucleated cells.

41. The method of embodiment 39 or 40, wherein the width of the constriction is about 4.2 μm to about 6 μm.

42. The method of any one of embodiments 39-41, wherein the width of the constriction is about 4.2 μm to about 4.8 μm.

43. The method of any one of embodiments 39-42, wherein the width of the constriction is about 4.5 μm.

44. The method of any one of embodiments 39-43, wherein the cell suspension comprising the plurality of input nucleated cells are passed through multiple constrictions wherein the multiple constrictions are arranged in series and/or in parallel.

45. The method of any one of embodiments 39-44, wherein the exogenous antigen is present in at least about 70% of the nucleated cells following incubation of the perturbed input nucleated cells with the exogenous antigen.

46. The method of any one of embodiments 1-45, wherein the nucleated cells are conditioned with an adjuvant to form conditioned cells.

47. The method of embodiment 46, wherein the nucleated cells are incubated with the adjuvant for about 1 hour to about 24 hours, about 2 hours to about 10 hours, about 3 hours to about 6 hours, or about 4 hours for the cells to condition.

48. The method of embodiment 46 or 47, wherein the nucleated cells are conditioned before or after introducing the exogenous antigen into the nucleated cells.

49. The method of any one of embodiments 46-48, wherein the adjuvant is a CpG oligodeoxynucleotide (ODN), LPS, IFN-α, STING agonists, RIG-I agonists, poly I.C, R837, R848, a TLR3 agonist, a TLR4 agonist or a TLR 9 agonist.

50. The method of any one of embodiments 46-49, wherein the adjuvant is a CpG oligodeoxynucleotide (ODN).

51. The method of any one of embodiments 46-50, wherein the adjuvant is CpG 7909.

52. The method of any one of embodiments 46-51, wherein the conditioned cells are a conditioned plurality of modified PBMCs.

53. The method of embodiment 52, wherein the plurality of modified PBMCs are further modified to increase expression of one or more of co-stimulatory molecules.

54. The method of embodiment 53, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

55. The method of any one of embodiments 52-54, wherein the plurality of modified PBMCs are further modified to increase expression of one or more cytokines.

56. The method of embodiment 55, wherein the cytokine is IL-15, IL-12, IL-2, IFN-α, or IL-21.

57. The method of any one of embodiment 52-56, wherein one or more co-stimulatory molecules is upregulated in the B cells of the conditioned plurality of modified PBMCs compared to the B cells in the plurality of nonmodified PBMCs, wherein the co-stimulatory molecule is CD80 and/or CD86.

58. The method of any one of embodiments 52-57, wherein the plurality of modified PBMCs have increased expression of one or more of IFN-γ, IL-6, MCP-1, MIP-1β, IP-10, or TNF-α compared to a plurality of unconditioned PBMCs.

59. The method of embodiment 58, wherein the expression of one or more of IFN-γ, IL-6, MCP-1, MIP-1β, IP-10, or TNF-α is increased by more than about 1.2-fold, 1.5-fold, 1.8-fold, 2-fold, 3-fold, 4-fold, 5-fold, 8-fold, or more than 10-fold compared to the plurality of unconditioned PBMCs.

60. The method of any one of embodiments 1-59, wherein the immunoconjugate is administered before, at the same time, or after administration of the composition comprising nucleated cells.

61. The method of any one of embodiments 1-60, wherein the composition comprising nucleated cells is administered a plurality of times.

62. The method of any one of embodiments 1-61, wherein the immunoconjugate is administered a plurality of times after administration of the composition comprising nucleated cells.

63. The method of any one of embodiments 1-62, wherein the composition and/or the immunoconjugate is administered intravenously.

64. The method of any one of embodiments 1-63, wherein the immunoconjugate is administered subcutaneously or intratumorally.

65. The method of embodiment 64, wherein the immunoconjugate is administered subcutaneously in combination with hyaluronidase.

66. The method of any one of embodiments 1-65, wherein the individual is a human.

67. The method of any one of embodiments 1-66, wherein the individual has cancer, an infectious disease, or a viral associated disease.

68. The method of any one of embodiments 1-67, wherein the composition of nucleated cells and/or the immunoconjugate is administered prior to, concurrently with, or following administration of another therapy.

69. The method of embodiment 68, wherein the another therapy is a chemotherapy or a radiation therapy.

70. A composition comprising nucleated cells comprising an exogenous antigen for use in a method for treating a disease in an individual, wherein the composition is used in combination with an immunoconjugate comprising a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19).

71. The composition of embodiment 70, wherein the disease is cancer, an infectious disease, or a viral-associated disease.

72. The composition of embodiment 70 or 71, wherein the composition comprising nucleated cells is administered before, at the same time, or after the immunoconjugate.

73. The composition of any one of embodiments 70-72, wherein the immunoconjugate is administered a plurality of times after administration of the composition comprising nucleated cells.

74. The composition of any one of embodiments 70-73, wherein the second polypeptide binds a T cell.

75. The composition of any one of embodiments 70-74, wherein the second polypeptide binds PD-1 expressed on the T cell.

76. The composition of any one of embodiments 70-75, wherein the second polypeptide is an antigen-binding moiety that specifically binds PD-1.

77. The composition of embodiment 76, wherein the anti-PD-1 antigen-binding moiety comprises

(a) a heavy chain variable region (VH) comprising a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6; or

(b) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO:13.

78. The composition of embodiment 76 or 77, wherein the anti-PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO:14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO:17, and SEQ ID NO:18.

79. The composition of any one of embodiments 76-78, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO:15, SEQ ID NO:16, SEQ ID NO: 17, and SEQ ID NO:18.

80. The composition of any one of embodiments 76-79, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO: 15.

81. The composition of any one of embodiments 70-80, wherein the immunoconjugate comprises a polypeptide comprising an amino acid sequence that is at least about 80%, 85%, 90%, 95%, 96%, 97%, 98%, 99% or 100% identical to the sequence of SEQ ID NO:24, a polypeptide comprising an amino acid sequence that is at least about 80%, 85%, 90%, 95%, 96%, 97%, 98%, 99% or 100% identical to the sequence of SEQ ID NO:24, and a polypeptide comprising an amino acid sequence that is at least about 80%, 85%, 90%, 95%, 96%, 97%, 98%, 99% or 100% identical to the sequence of SEQ ID NO:25.

82. The composition of any one of embodiments 70-81, wherein the immunoconjugate comprises a polypeptide sequence of SEQ ID NO:22, a polypeptide sequence of SEQ ID NO:24, and a polypeptide sequence of SEQ ID NO:25.

83. The composition of any one of embodiments 70-73, wherein the second polypeptide specifically binds a target antigen presented on a tumor cell or in a tumor cell environment.

84. The composition of embodiment 83, wherein the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP).

85. The composition of any one of embodiments 70-73 and 83-84, wherein the second polypeptide binds FAP.

86. The composition of any one of embodiments 70-73 and 83-85, wherein the second polypeptide is an antigen-binding moiety that specifically binds FAP.

87. The composition of any one of embodiments 70-73 and 83-86, wherein the antigen-binding moiety the specifically bind FAP comprising (i) the heavy chain variable region sequence of SEQ ID NO:29 and the light chain variable region sequence of SEQ ID NO: 28; (ii) the heavy chain variable region sequence of SEQ ID NO:31 and the light chain variable region sequence of SEQ ID NO:30; (iii) the heavy chain variable region sequence of SEQ ID NO: 33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36.

88. The composition of any one of embodiments 70-73 and 83-87, wherein the antigen-binding moiety the specifically bind FAP comprises (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO: 231; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41.

89. The composition of any one of embodiments 70-73 and 83-88, wherein antigen-binding moiety the specifically bind FAP comprises the heavy chain variable region sequence of SEQ ID NO:33 and the light chain variable region sequence of SEQ ID NO:32.

90. The composition of any one of embodiments 70-73 and 83-89, wherein the immunoconjugate comprises a polypeptide sequence of SEQ ID NO:38, a polypeptide sequence of SEQ ID NO:39, and a polypeptide sequence of SEQ ID NO:40.

91. The composition of any one of embodiments 70-90, wherein the mutant IL-2 polypeptide further comprises the amino acid substitution T3A and/or the amino acid substitution C125A.

92. The composition of any one of embodiments 70-91, wherein the mutant IL-2 polypeptide comprises an amino acid sequence that is at least about 95%, 96%, 97%, 98%, 99% or 100% identical to the amino acid sequence of SEQ ID NO:20, wherein the mutant IL-2 polypeptide shows reduced affinity to the high-affinity IL-2 receptor and substantially similar affinity to the intermediate-affinity IL-2 receptor, each compared to a wild-type IL-2 polypeptide.

93. The composition of any one of embodiments 70-92, wherein the mutant IL-2 polypeptide comprises the amino acid sequence of SEQ ID NO:20.

94. The composition of any one of embodiments 70-93, wherein the nucleated cells are immune cells.

95. The composition of any one of embodiments 70-94, wherein the nucleated cells are a plurality of peripheral blood mononuclear cells (PBMCs).

96. The composition of embodiment 95, wherein the plurality of PBMCs comprise two or more of T cell, B cell, NK cell, monocytes, dendritic cells or NK-T cells.

97. The composition of any one of embodiments 70-94, wherein the nucleated cells are one or more of T cells, B cells, NK cells, monocytes, dendritic cells and/or NK-T cells.

98. The composition of any one of embodiments 70-97, wherein the exogenous antigen is delivered to the nucleated cells intracellularly.

99. The composition of any one of embodiments 70-98, wherein the exogenous antigen is a disease-associated antigen.

100. The composition of any one of embodiments 70-99, wherein the disease-associated antigen is a cancer antigen, an infectious disease antigen, or a viral-associated disease antigen.

101. The composition of any one of embodiments 70-100, wherein the exogenous antigen is a human papillomavirus (HPV) antigen.

102. The composition of any one of embodiments 70-101, wherein the composition further comprises an adjuvant.

103. The composition of embodiment 102, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

104. The composition of any one of embodiments 70-103, wherein the nucleated cells comprising the exogenous antigen are prepared by a process comprising the steps of:

a) passing a cell suspension comprising input nucleated cells through a cell-deforming constriction, wherein a diameter of the constriction is a function of a diameter of the input nucleated cells in the suspension, thereby causing perturbations of the input nucleated cells large enough for the exogenous antigen to pass through to form perturbed input nucleated cells;

b) incubating the perturbed input nucleated cells with the exogenous antigen for a sufficient time to allow the exogenous antigen to enter the perturbed input nucleated cells, thereby generating nucleated cells comprising the exogenous antigen.

105. The composition of embodiment 104, wherein the width of the constriction is about 10% to about 99% of the mean diameter of the input nucleated cells.

106. The composition of embodiment 104 or 105, wherein the width of the constriction is about 4.2 μm to about 6 μm.

107. The composition of any one of embodiments 104-106, wherein the width of the constriction is about 4.2 μm to about 4.8 μm.

108. The composition of any one of embodiments 104-107, wherein the width of the constriction is about 4.5 μm.

109. The composition of any one of embodiments 104-108, wherein the cell suspension comprising the plurality of input nucleated cells are passed through multiple constrictions wherein the multiple constrictions are arranged in series and/or in parallel.

110. The composition of any one of embodiments 104-109, wherein the exogenous antigen is present in at least about 70% of the nucleated cells following incubation of the perturbed input nucleated cells with the exogenous antigen.

111. The composition of any one of embodiments 70-110, wherein the nucleated cells are conditioned with an adjuvant to form conditioned cells.

112. The composition of embodiment 111, wherein the nucleated cells are incubated with the adjuvant for about 1 hour to about 24 hours, about 2 hours to about 10 hours, about 3 hours to about 6 hours, or about 4 hours for the cells to condition.

113. The composition of embodiment 111 or 112, wherein the nucleated cells are conditioned before or after introducing the exogenous antigen into the nucleated cells.

114. The composition of any one of embodiments 111-113, wherein the adjuvant is a CpG oligodeoxynucleotide (ODN), LPS, IFN-α, STING agonists, RIG-I agonists, poly I.C, R837, R848, a TLR3 agonist, a TLR4 agonist or a TLR 9 agonist.

115. The composition of any one of embodiments 111-114, wherein the adjuvant is a CpG oligodeoxynucleotide (ODN).

116. The composition of any one of embodiments 111-115, wherein the adjuvant is CpG 7909.

117. The composition of any one of embodiments 111-116, wherein the conditioned cells are a conditioned plurality of modified PBMCs.

118. The composition of embodiment 117, wherein the plurality of modified PBMCs are further modified to increase expression of one or more of co-stimulatory molecules.

119. The composition of embodiment 118, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

120. The composition of any one of embodiments 117-119, wherein the plurality of modified PBMCs are further modified to increase expression of one or more cytokines.

121. The composition of embodiment 120, wherein the cytokine is IL-15, IL-12, IL-2, IFN-α, or IL-21.

122. The composition of any one of embodiments 117-123, wherein one or more co-stimulatory molecules is upregulated in the B cells of the conditioned plurality of modified PBMCs compared to the B cells in the plurality of nonmodified PBMCs, wherein the co-stimulatory molecule is CD80 and/or CD86.

123. The composition of any one of embodiments 117-124, wherein the plurality of modified PBMCs have increased expression of one or more of IFN-γ, IL-6, MCP-1, MIP-1β, IP-10, or TNF-α compared to a plurality of unconditioned PBMCs.

124. The composition of any one of embodiments 117-123, wherein the expression of one or more of IFN-γ, IL-6, MCP-1, MIP-1β, IP-10, or TNF-α is increased by more than about 1.2-fold, 1.5-fold, 1.8-fold, 2-fold, 3-fold, 4-fold, 5-fold, 8-fold, or more than 10-fold compared to the plurality of unconditioned PBMCs.

125. The composition of any one of embodiments 70-124, wherein the composition and/or the immunoconjugate is administered intravenously.

126. The composition of any one of embodiments 70-125, wherein the immunoconjugate is administered subcutaneously or intratumorally.

127. The composition of embodiment 126, wherein the immunoconjugate is administered subcutaneously in combination with hyaluronidase.

128. The composition of any one of embodiments 70-127, wherein the individual is a human.

129. The composition of any one of embodiments 70-128, wherein the individual has cancer, an infectious disease, or a viral associated disease.

130. The composition of any one of embodiments 70-129, wherein the composition is administered prior to, concurrently with, or following administration of another therapy.

131. The composition of embodiment 130, wherein the another therapy is a chemotherapy or a radiation therapy.

132. An immunoconjugate comprising a mutant IL-2 polypeptide and a second polypeptide capable of specific binding to a T cell, a tumor cell, or the tumor cell environment for use in a method for treating a disease in an individual, wherein the immunoconjugate is used in combination with a composition comprising nucleated cells comprising an exogenous antigen.

133. The immunoconjugate of embodiment 132, wherein the disease is cancer, an infectious disease, or a viral-associated disease.

134. The immunoconjugate of embodiment 132 or 133, wherein the composition comprising the nucleated cells is administered before, at the same time, or after the immunoconjugate.

135. The immunoconjugate of any one of embodiments 132-134, wherein the immunoconjugate is administered a plurality of times after administration of the composition comprising nucleated cells.

136. The immunoconjugate of any one of embodiments 132-135, wherein the second polypeptide binds a T cell.

137. The immunoconjugate of any one of embodiments 132-136, wherein the second polypeptide binds PD-1 expressed on the T cell.

138. The immunoconjugate of any one of embodiments 132-137, wherein the second polypeptide is an antigen-binding moiety that specifically binds PD-1.

139. The immunoconjugate of embodiment 138, wherein the anti-PD-1 antigen-binding moiety comprises

(a) a heavy chain variable region (VH) comprising a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6; or

(b) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO: 13.

140. The immunoconjugate of embodiment 138 or 139, wherein the anti-PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO: 14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO:17, and SEQ ID NO:18.

141. The immunoconjugate of any one of embodiments 138-140, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO:15, SEQ ID NO:16, SEQ ID NO: 17, and SEQ ID NO:18.

142. The immunoconjugate of any one of embodiments 138-141, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO: 15.

143. The immunoconjugate of any one of embodiments 132-142, wherein the immunoconjugate comprises a polypeptide comprising an amino acid sequence that is at least about 80%, 85%, 90%, 95%, 96%, 97%, 98%, 99% or 100% identical to the sequence of SEQ ID NO:22, a polypeptide comprising an amino acid sequence that is at least about 80%, 85%, 90%, 95%, 96%, 97%, 98%, 99% or 100% identical to the sequence of SEQ ID NO:24, and a polypeptide comprising an amino acid sequence that is at least about 80%, 85%, 90%, 95%, 96%, 97%, 98%, 99% or 100% identical to the sequence of SEQ ID NO:25.

144. The immunoconjugate of any one of embodiments 132-143, wherein the immunoconjugate comprises a polypeptide sequence of SEQ ID NO:22, a polypeptide sequence of SEQ ID NO:24, and a polypeptide sequence of SEQ ID NO:25.

145. The immunoconjugate of any one of embodiments 132-135, wherein the second polypeptide specifically binds a target antigen presented on a tumor cell or in the tumor cell environment.

146. The immunoconjugate of any one of embodiments 132-135 and 145, wherein the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), the Carcinoembryonic Antigen (CEA) and the Melanoma-associated Chondroitin Sulfate Proteoglycan (MCSP).

147. The immunoconjugate of any one of embodiments 132-135 and 145-146, wherein the second polypeptide binds FAP.

148. The immunoconjugate of any one of embodiments 132-135 and 145-147, wherein the second polypeptide is an antigen-binding moiety that specifically binds FAP.

149. The immunoconjugate of embodiment 148, wherein the antigen-binding moiety the specifically bind FAP comprising (i) the heavy chain variable region sequence of SEQ ID NO:29 and the light chain variable region sequence of SEQ ID NO: 28; (ii) the heavy chain variable region sequence of SEQ ID NO:31 and the light chain variable region sequence of SEQ ID NO:30; (iii) the heavy chain variable region sequence of SEQ ID NO: 33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36.

150. The immunoconjugate of embodiment 148 or 149, wherein the antigen-binding moiety the specifically bind FAP comprising (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO: 231; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41.

151. The immunoconjugate of any one of embodiments 148-150, wherein antigen-binding moiety the specifically bind FAP comprises the heavy chain variable region sequence of SEQ ID NO:33 and the light chain variable region sequence of SEQ ID NO:32.

152. The immunoconjugate of any one of embodiments 132-135 and 145-151, wherein the immunoconjugate comprises a polypeptide sequence of SEQ ID NO: 38, a polypeptide sequence of SEQ ID NO:39, and a polypeptide sequence of SEQ ID NO:40.

153. The immunoconjugate of any one of embodiments 132-152, wherein the mutant IL-2 polypeptide further comprises the amino acid substitution T3A and/or the amino acid substitution C125A.

154. The immunoconjugate of any one of embodiments 132-153, wherein the mutant IL-2 polypeptide comprises an amino acid sequence that is at least about 95%, 96%, 97%, 98%, 99% or 100% identical to the amino acid sequence of SEQ ID NO:20, wherein the mutant IL-2 polypeptide shows reduced affinity to the high-affinity IL-2 receptor and substantially similar affinity to the intermediate-affinity IL-2 receptor, each compared to a wild-type IL-2 polypeptide.

155. The immunoconjugate of any one of embodiments 132-154, wherein the mutant IL-2 polypeptide comprises the amino acid sequence of SEQ ID NO:20.

156. The immunoconjugate of any one of embodiments 132-155, wherein the nucleated cells are immune cells.

157. The immunoconjugate of any one of embodiments 132-156, wherein the nucleated cells are a plurality of peripheral blood mononuclear cells (PBMCs).

158. The immunoconjugate of embodiment 157, wherein the plurality of PBMCs comprise two or more of T cell, B cell, NK cell, monocytes, dendritic cells or NK-T cells.

159. The immunoconjugate of any one of embodiments 132-156, wherein the nucleated cells are one or more of T cells, B cells, NK cells, monocytes, dendritic cells and/or NK-T cells.

160. The immunoconjugate of any one of embodiments 132-159, wherein the exogenous antigen is delivered to the nucleated cells intracellularly.

161. The immunoconjugate of any one of embodiments 132-160, wherein the exogenous antigen is a disease-associated antigen.

162. The immunoconjugate of any one of embodiments 132-161, wherein the disease-associated antigen is a cancer antigen, an infectious disease antigen, or a viral-associated disease antigen.

163. The immunoconjugate of any one of embodiments 132-162, wherein the exogenous antigen is a human papillomavirus (HPV) antigen.

164. The immunoconjugate of any one of embodiments 132-163, wherein the composition further comprises an adjuvant.

165. The immunoconjugate of embodiment 164, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

166. The immunoconjugate of any one of embodiments 132-165, wherein the nucleated cells comprising the exogenous antigen are prepared by a process comprising the steps of:

a) passing a cell suspension comprising input nucleated cells through a cell-deforming constriction, wherein a diameter of the constriction is a function of a diameter of the input nucleated cells in the suspension, thereby causing perturbations of the input nucleated cells large enough for the exogenous antigen to pass through to form perturbed input nucleated cells;

b) incubating the perturbed input nucleated cells with the exogenous antigen for a sufficient time to allow the exogenous antigen to enter the perturbed input nucleated cells, thereby generating nucleated cells comprising the exogenous antigen.

167. The immunoconjugate of embodiment 166, wherein the width of the constriction is about 10% to about 99% of the mean diameter of the input nucleated cells.

168. The immunoconjugate of embodiment 166 or 167, wherein the width of the constriction is about 4.2 μm to about 6 μm.

169. The immunoconjugate of any one of embodiments 166-168, wherein the width of the constriction is about 4.2 μm to about 4.8 μm.

170. The immunoconjugate of any one of embodiments 166-169, wherein the width of the constriction is about 4.5 μm.

171. The immunoconjugate of any one of embodiments 166-170, wherein the cell suspension comprising the plurality of input nucleated cells are passed through multiple constrictions wherein the multiple constrictions are arranged in series and/or in parallel.

172. The immunoconjugate of any one of embodiments 166-171, wherein the exogenous antigen is present in at least about 70% of the nucleated cells following incubation of the perturbed input nucleated cells with the exogenous antigen.

173. The immunoconjugate of any one of embodiments 132-172, wherein the nucleated cells are conditioned with an adjuvant to form conditioned cells.

174. The immunoconjugate of embodiment 173, wherein the nucleated cells are incubated with the adjuvant for about 1 hour to about 24 hours, about 2 hours to about 10 hours, about 3 hours to about 6 hours, or about 4 hours for the cells to condition.

175. The immunoconjugate of embodiment 173 or 174, wherein the nucleated cells are conditioned before or after introducing the exogenous antigen into the nucleated cells.

176. The immunoconjugate of any one of embodiments 173-175, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

177. The immunoconjugate of any one of embodiments 173-176, wherein the adjuvant is a CpG oligodeoxynucleotide (ODN).

178. The immunoconjugate of any one of embodiments 173-177, wherein the adjuvant is CpG 7909.

179. The immunoconjugate of any one of embodiments 173-178, wherein the conditioned cells are a conditioned plurality of modified PBMCs.

180. The immunoconjugate of embodiment 179, wherein the plurality of modified PBMCs are further modified to increase expression of one or more of co-stimulatory molecules.

181. The immunoconjugate of embodiment 180, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

182. The immunoconjugate of any one of embodiments 179-181, wherein the plurality of modified PBMCs are further modified to increase expression of one or more cytokines.

183. The immunoconjugate of embodiment 182, wherein the cytokine is IL-15, IL-12, IL-2, IFN-α, or IL-21.

184. The immunoconjugate of any one of embodiments 179-183, wherein one or more co-stimulatory molecules is upregulated in the B cells of the conditioned plurality of modified PBMCs compared to the B cells in the plurality of nonmodified PBMCs, wherein the co-stimulatory molecule is CD80 and/or CD86.

185. The immunoconjugate of any one of embodiments 179-184, wherein the plurality of modified PBMCs have increased expression of one or more of IFN-γ, IL-6, MCP-1, MIP-1β, IP-10, or TNF-α compared to a plurality of unconditioned PBMCs.

186. The immunoconjugate of any one of embodiments 179-185, wherein the expression of one or more of IFN-γ, IL-6, MCP-1, MIP-1β, IP-10, or TNF-α is increased by more than about 1.2-fold, 1.5-fold, 1.8-fold, 2-fold, 3-fold, 4-fold, 5-fold, 8-fold, or more than 10-fold compared to the plurality of unconditioned PBMCs.

187. The immunoconjugate of any one of embodiments 132-186, wherein the composition and/or the immunoconjugate is administered intravenously.

188. The immunoconjugate of any one of embodiments 132-186, wherein the immunoconjugate is administered subcutaneously or intratumorally.

189. The immunoconjugate of embodiment 188, wherein the immunoconjugate is administered subcutaneously in combination with hyaluronidase.

190. The immunoconjugate of any one of embodiments 132-189, wherein the individual is a human.

191. The immunoconjugate of any one of embodiments 132-190, wherein the individual has cancer, an infectious disease, or a viral associated disease.

192. The immunoconjugate of any one of embodiments 132-191, wherein the composition is administered prior to, concurrently with, or following administration of another therapy.

193. The immunoconjugate of embodiment 192, wherein the another therapy is a chemotherapy or a radiation therapy.

194. Use of an effective amount of an immunoconjugate in the manufacture of a medicament for stimulating an immune response in an individual, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO. 19);

wherein the immunoconjugate is formulated for administration in conjunction with a composition comprising nucleated cells, wherein the nucleated cells comprise an exogenous antigen.

195. The use of embodiment 194, wherein the immunoconjugate is administered before, at the same time, or after the composition comprising nucleated cells.

196. Use of an effective amount of a composition in the manufacture of a medicament for stimulating an immune response in an individual, wherein the composition comprises nucleated cells comprising an exogenous antigen;

wherein the composition is formulated for administration in conjunction with an immunoconjugate comprising a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19).

197. The composition of embodiment 196, wherein the composition comprising the nucleated cells is administered before, at the same time, or after the immunoconjugate.

198. A kit for use in the method of any one of embodiments 1-69.

199. A kit comprising a composition of nucleated cells comprising an exogenous antigen and an immunoconjugate, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19); wherein the composition and the immunoconjugate are for use in combination for stimulating an immune response to the exogenous antigen in an individual.

200. A kit comprising a composition of nucleated cells comprising an exogenous antigen, wherein the composition is for use in conjunction with an immunoconjugate for stimulating an immune response to the exogenous antigen in an individual;

wherein the immunoconjugate comprises a mutant IL-2 polypeptide and a second polypeptide capable of specific binding to a T cell, a tumor cell, or the tumor cell environment; and wherein the mutant IL-2 polypeptide is a human IL-2 molecule comprising the amino acid substitutions F42A, Y45A and L72G (numbering relative to the human IL-2 sequence SEQ ID NO: 19).

201. A kit comprising an immunoconjugate, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19);

wherein the immunoconjugate is for use in conjunction with a composition of nucleated cells comprising an exogenous antigen for stimulating an immune response to the exogenous antigen in an individual.

202. A method for producing an immunoconjugate for use in conjunction with a composition comprising nucleated cells for stimulating an immune response in an individual, the method comprising expressing a nucleic acid encoding the immunoconjugate in a cell under conditions to produce the immunoconjugate,

wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19);

wherein the immunoconjugate is for use in conjunction with administering a composition comprising nucleated cells, wherein the nucleated cells comprise an exogenous antigen.

203. The method of embodiment 202, wherein the immunoconjugate is a fusion protein.

204. A method for producing a composition comprising nucleated cells for use in conjunction with an immunoconjugate for stimulating an immune response in an individual, the method comprising introducing an exogenous antigen intracellularly to a population of nucleated cells;

wherein the composition is for use in conjunction with administration of an immunoconjugate;

wherein the immunoconjugate comprises a mutant IL-2 polypeptide and a second polypeptide capable of specific binding to a T cell, a tumor cell, or the tumor cell environment; and wherein the mutant IL-2 polypeptide is a human IL-2 molecule comprising the amino acid substitutions F42A, Y45A and L72G (numbering relative to the human IL-2 sequence SEQ ID NO: 19).

### EXAMPLES

Those skilled in the art will recognize that several embodiments are possible within the scope and spirit of this invention. The invention will now be described in greater detail by reference to the following non limiting examples. The following examples further illustrate the invention but, of course, should not be construed as in any way limiting its scope.

In the following examples, SQZ-PBMC refers to composition of conditioned PCMCs comprising an exogenous antigen which is the HPV16 E7(43-77) SLP (SEQ ID NO:56) delivered intracellularly using SQZ technology as described herein. PD1-IL2v refers to an immunoconjugate comprising a variant IL-2 polypeptide and a PD-1 antigen-binding moiety. FAP-IL2v refers to an immunoconjugate comprising a variant IL-2 polypeptide and a FAP antigen-binding moiety

### Example 1. SQZ-PBMC Treatment in Combination with PD1-IL2v has Synergistic Benefit at all Cell Doses

TC1 cells (kindly provided by Prof TC Wu, Johns Hopkins University, Baltimore, Md.), were implanted subcutaneously on the flank of C57BL/6NCrl mice (purchased from Charles River) at a concentration of 5×10⁵ cells in 100 μL HBBS. Tumor growth was measured with a caliper using the formula V=W²Lπ/6. Animal experiments were conducted under the approved license VD3142.

Mice containing TC1 tumor implants were used to test the effectiveness of combination therapy of SQZ-PBMC, comprising an E7 HPV antigen delivered intracellularly, and PD1-IL2v. The combined treatment was administered to mice intravenously on day 7, 10, and 13 TC1 cell implantation. Tumor volume was measured at regular intervals around 2-3 times per week between 10-60 days post tumor growth onset. Survival was monitored daily, and tumor growth was measured twice a week. Tumor length (mm) and width (mm) was measured with calipers, and tumor volume (mm³) was computed as the product of tumor length (mm)×width (mm)×width (mm). Body weight of each mouse were measured twice per week and compared to a starting body weight prior to tumor implantation. Each group of mice received a fixed dose of a mouse surrogate of SQZ-PBMC treatment as described in Table 1 below. Mice that received a combination therapy of SQZ-PBMC were given the same doses of cells together with a fixed dose of PD1-IL2v.

TABLE 1
Treatment Groups and Dosages of Therapeutics

| Control | No treatment |
|---|---|
| PD1-IL2v | 0.5 mg/kg PD1-IL2v |
| SQZ-PMBC | 0.25 × 10⁶ SQZ-PBMC cells |
| SQZ-PMBC | 1 × 10⁶ SQZ-PBMC cells |
| SQZ-PMBC | 4 × 10⁶ SQZ- PBMC cells |
| SQZ-PMBC + PD1-IL2v | 0.25 × 10⁶ SQZ-PBMC cells + |
| | 0.5 mg/kg PD1-IL2v |
| SQZ-PMBC + PD1-IL2v | 1 × 10⁶ SQZ- PBMC cells + |
| | 0.5 mg/kg PD1-IL2v |
| SQZ-PMBC + PD1-IL2v | 4 × 10⁶ SQZ- PBMC cells + |
| | 0.5 mg/kg PD1-IL2v |

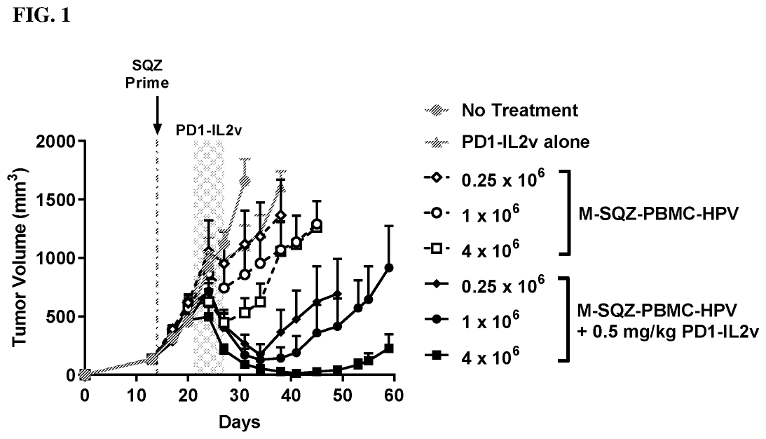

According to FIG. 1, PD1-IL2v treatment or low concentrations of SQZ-PBMC were effective at reducing tumor volume relative to no treatment following about 27 days post-administration. The effectiveness of SQZ-PBMC alone increased with cell dose.

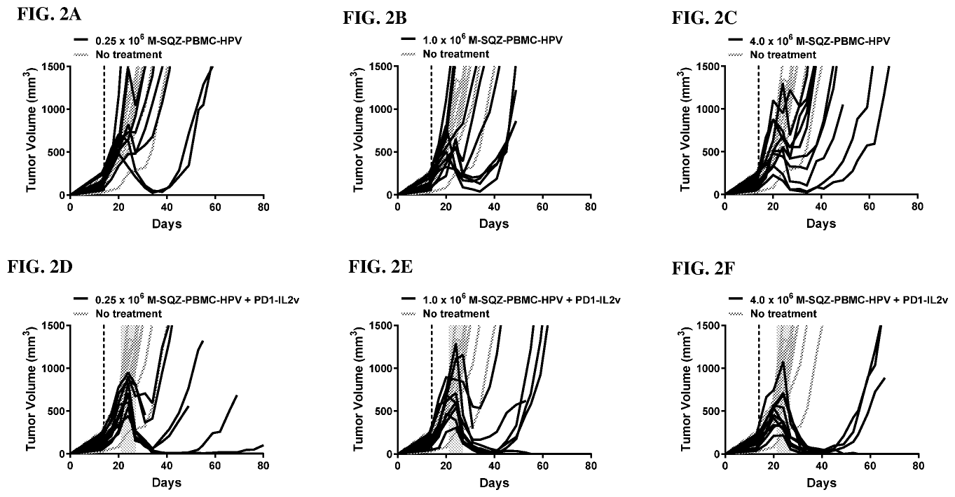

Mice were given SQZ-PBMC 14 days post tumor implantation, followed by three administrations of PD1-IL2v on days 21, 24, and 27 post-tumor implantation. The strongest tumor reduction volume was seen in therapies combining SQZ-PBMC with PD1-IL2v (FIG. 1). Mice receiving doses of SQZ-PBMC alone showed an immediate decrease in tumor volume. When combined with PD1-IL2v, 6 of 10 mice were tumor free at 12 weeks in the 4×10⁶ SQZ-PBMC cell dose (FIG. 1, FIG. 2F). Additionally, 3 of 10 mice and 1 of 10 mice were tumor free in the 1×10⁶ (FIG. 1, FIG. 2E) and 0.25×10⁶ (FIG. 1, FIG. 2D) SQZ combined cell dosages, respectively.

The beneficial effects of PD1-IL2v on tumor clearance was not limited to molecules targeting PD1. Additional testing was performed by treatment with FAP-IL2v and measuring resultant tumor volume using similar methods. Experiments were run with another cohort (Table 2). In this set of experiments, 12 mice were used for all treatment groups, which includes no treatment, SQZ-PBMC (1.0×10⁶ cells), SQZ+FAP-IL2v (1.0×10⁶ cells+2 mg/kg), FAP-IL2v (2 mg/kg), SQZ+PD1-IL2v (1.0×10⁶ cells+1 mg/kg), and PD1-IL2v (1 mg/kg). Mice receiving SQZ treatment received I.V. injections on day 14, and additional I.V. injections of FAP-IL2v or PD1-IL2v were performed on day 21, 28, and 35 for combination dosing groups.

TABLE 2
Combined treatment groups

| | | Combination | Number |
|---|---|---|---|
| Groups | SQZ-PBMC | Dosing | of Mice |
| No treatment | — | — | 12 |
| SQZ-PBMC | 1.0 × 10⁶ cells | — | 12 |
| SQZ-PBMC + FAP-IL2v | 1.0 × 10⁶ cells | 2 mg/kg | 12 |
| FAP-IL2v | — | 2 mg/kg | 12 |
| SQZ-PBMC + PD1-IL2v | 1.0 × 10⁶ cells | 1 mg/kg | 11 |
| PD1-IL2v | — | 1 mg/kg | 12 |

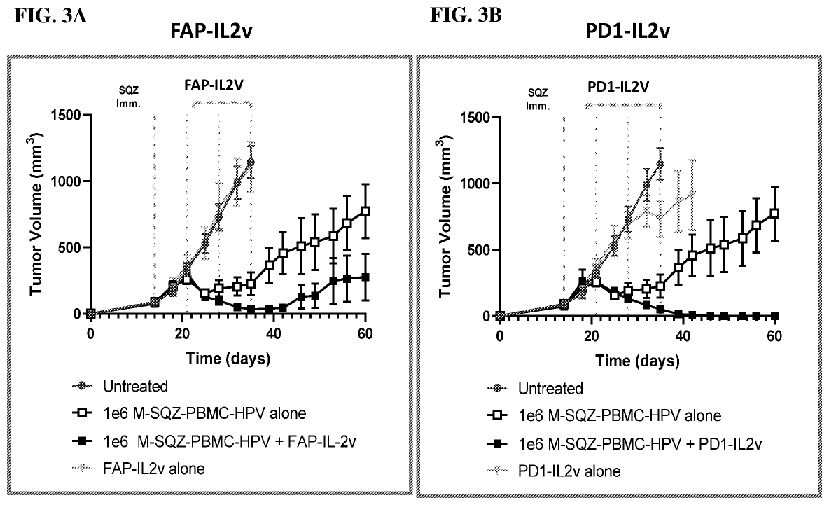

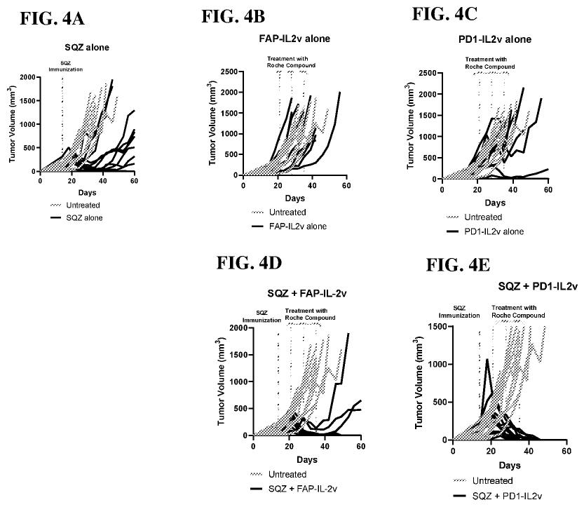

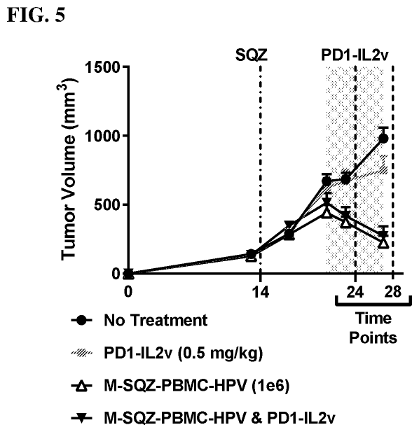

In both treatment groups, FAP-IL2v or PD1-IL2v alone had moderate or no effects on the tumor volume in mice relative to controls not receiving any form of treatment (FIG. 3A-B). Treatment with SQZ-PBMC alone is plotted on both figures, showing that PBMC itself is effective at reducing tumor volume over the course of many weeks following treatment. When the combination of either SQZ-PBMC with FAP-IL2v or PD1-IL2v is delivered, tumor volume was dramatically decreased following treatment, and the suppressive effect persisted on the time scale of months. It was found that 75% of all mice treated with the combination of SQZ-PBMC+FAP-IL2v were tumor-free on day 49 of the experiment and 100% of all mice treated with SQZ-PBMC+PD1-IL2v were tumor free at the same timepoint of the experiment. In contrast, 17% of mice receiving SQZ-PBMC treatment only were tumor free at the end of the experiment. Spider plots showing tumor volumes for individual mice are shown in FIG. 4, highlighting the consistent tumor volume measurements across individual mice. Mice receiving SQZ-PBMC and the combined therapy of SQZ-PBMC+PD1-IL2v both showed a decrease in tumor volume around day 24, and the tumor volume continued to decrease for the duration of the experiment through day 28 (FIG. 5).

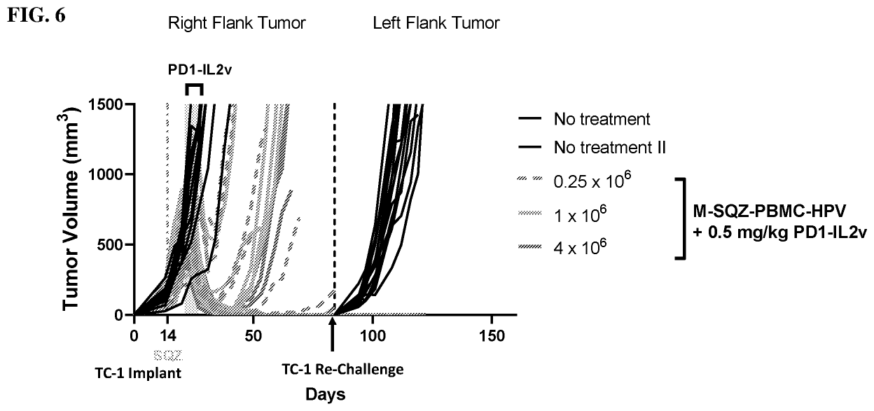

Lastly, the long-term effects on tumor formation were assessed by re-introducing tumors into mice. Tumor-free mice were re-challenged with left flank tumors 12 weeks after the primary tumor implant. Tumor volume of the left flank tumor was assessed following TC-1 tumor re-challenge for mice receiving no treatment or receiving a combined treatment of 0.25×10⁶, 1×10⁶ or 4×10⁶ SQZ-PBMC with 0.5 mg/kg PD1-IL2v. Mice were resistant to tumor rechallenge following treatment with SQZ-PBMC and PD1-IL2v (FIG. 6).

### Example 2. Impact of PD1-IL2v on SQZ-PBMC Treatment Using TIL Analysis

Tumor-infiltrating leukocytes (TIL) analysis was performed on tumor samples to determine the impact of PD1-IL2v on SQZ-PBMC treatment efficacy. Mice were divided into four treatment groups including a control (receiving no treatment), a PD1-IL2v only treatment group (receiving 0.5 mg/kg of PD1-IL2v), a SQZ-only treatment group (receiving SQZ-PBMC dosed at 1×10⁶), and a combined treatment group of SQZ-PBMC+PD1-IL2v (receiving 1×10⁶ SQZ-PBMC with 0.5 mg/kg PD1-IL2v).

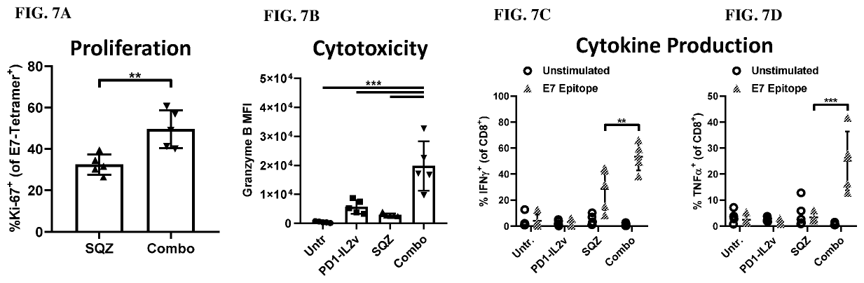

SQZ-PBMC was administered on day 14, and PDL1-IL2v was administered on days 21, 24, and 27. Following treatment, the percentage of Ki67+ expression within E7-tetramer cells was used to quantify T-cell proliferation within tumors. The total percentage of Ki67+ expression was significantly greater following treatment with the combination of SQZ-PBMC+PDL1-IL2v compared to treatment with SQZ-PBMC alone (FIG. 7A). Cytotoxicity was quantified by assessing the total granzyme B mean fluorescence intensity (MFI) within tumor samples. Granzyme B is secreted by NK cells and T-cells during an immune response, and high levels of granzyme B are related to the induction of an immune response. Combined treatment with SQZ-PBMC+PD1-IL2v resulted in the largest granzyme B MFI compared to all treatment groups. PD1-IL2v and SQZ-PBMC treatment alone induced a slight increase in granzyme B MFI compared to untreated controls (FIG. 7B). Next, cytokine production was measured by quantifying percentage of interferon gamma (IFN-γ) and tumor necrosis factor (TNF) expression on CD8+ cells. Percentage of cytokine-expressing CD8+ T-cells were quantified across all treatment groups in unstimulated and E7-stimulated epitopes. Cytokine production was significantly increased in E7 epitope tumor samples relative to unstimulated samples following combined treatment (FIGS. 7C-7D). This suggests that targeted cytotoxic T-cells are specifically recruited to the tumors. The cytokine levels were low in all other conditions.

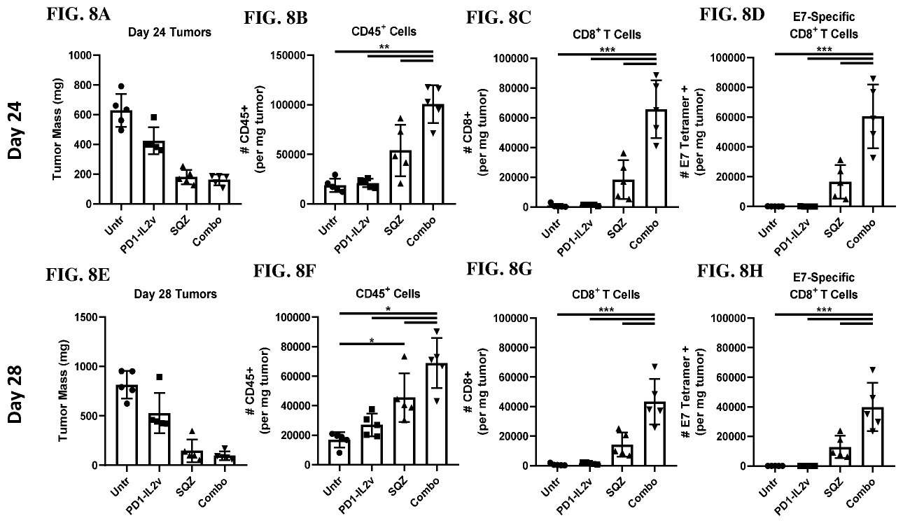

Tumor mass was quantified in day 24 (FIG. 8A) and day 28 (FIG. 8E). By day 24 and 28, tumors were comparable in size for groups receiving SQZ-PBMC alone or in combination with PD1-IL2v (FIGS. 8A, 8E). It is worth noting that day 28 is too early to see a difference between SQZ-PBMC and combined therapy with PD1-IL2v. SQZ-PBMC and combined therapy of SQZ-PBMC with PD1 resulted in the largest decrease in tumor mass in mice. Additionally, PD1-IL2v increased intratumoral E7-specific CD8+ T cells by over 3-fold versus SQZ-PBMC alone (FIGS. 8C, 8G).

The number of CD45+ cells and CD8+ cells per mg of tumor were also quantified across treatment groups using flow cytometry. CD45, a marker of immune cells can be used to assess the extent of an immune infiltration in a given tissue. Combination therapy of SQZ-PBMC with PD1-IL2v resulted in the largest density of CD45+ cells per tumor compared to other treatment groups (FIGS. 8B, 8F). The increase of CD45+ cells in tumors of subjected to combination therapy mice suggests that the therapy aids in engaging immunity that recognizes and removes the tumor.

Next, CD8+ cells per tumor was quantified across treatment groups. As before, the combined treatment of SQZ-PBMC with PD1-IL2v resulted in the greatest amount of CD8+ cells per tumor (FIG. 8C, G). Untreated and PD1-IL2v treated groups had nearly zero CD8+ cells per tumor, suggesting the PD1-IL2v alone does not lead to a recruitment of CD8+ cells into the tumor. SQZ-PBMC treatment alone had a substantial increase in CD8+ expression relative to untreated and PD1-IL2v treatment groups. The results suggest that SQZ-PBMC was effective at recruiting CD8+ cells, but the combination of SQZ-PBMC with PD1-IL2v was most effective at recruiting CD8+ cells to the tumor (FIG. 8C, G). Lastly, E7 tetramer+ cells per tumor mass was quantified for all treatment groups on day 24 and 28 by quantifying E7 tetramer cell expression via flow cytometry. Untreated and PD1-IL2v treated mice had zero quantified E7-specific CD8 T cells. SQZ-PBMC treated mice had significantly greater E7-specific cells than untreated and PD1-IL2v treated. The combined therapy of SQZ-PBMC with PD1-IL2v resulted in an increase in E7-specific CD8+ T cells that was significantly larger than any other treatment group (FIG. 8D, H). This suggests that combination therapy of SQZ-PBMC with PD1-IL2v is most effective at recruiting E7-specific CD8 T cells in tumors in mice and the immune response is antigen-specific.

### Example 3. The Impact of PD1-IL2v Treatment Appears Equivalent for Antigen-Specific and Non-Specific CD8 T-Cells

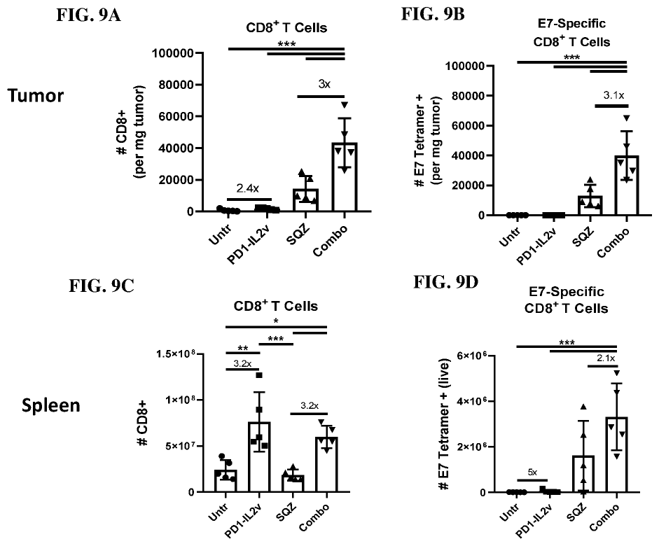

The total amount of immune cells were quantified within tumors and spleens of mice across all treatment groups. Under normal conditions, tumors typically do not have immune cells, whereas the spleen has baseline amounts of immune cells. As a way to determine the efficacy of combined therapy towards increasing the total amount of immune cells within the tumor cell environment, CD8+ cells and E7 tetramer-specific T cells were compared within tumors and spleens of mice across all treatment groups, including untreated, PD1-IL2v-treated, SQZ-PBMC-treated, and combined PD1-IL2v-treated and SQZ-PBMC-treated mice. As expected, untreated mice had no detectable levels of CD8+ in tumors (FIGS. 9A, 9B), whereas the spleen had a measurable amount of CD8+ cells in untreated groups (FIG. 9C), but did not have E7 tetramer-specific CD8+ T cells (FIG. 9D).

Similarly, the use of PD1-IL2v treatment alone did not result in recruitment of CD8+(FIG. 9A) or E7-specific CD8+ T cells (FIG. 9C) within the tumor. The spleen had 3.2× more CD8+ T cells in PD1-IL2v-treated mice than in untreated mice (FIG. 9C), suggesting that PD1-IL2v treatment alone is capable of increasing circulating systemic immune cells. Together, the data suggests that PD1-IL2v alone is capable of increasing the total amount of immune cells, but it alone is not sufficient to increase E7 tetramer specific CD8+ T cells within tumors. In the spleen, PD1 treatment also did not contribute to an increase in E7 tetramer-specific T cells, suggesting that the mice are not developing HPV-specific immune cells in this single treatment regime (FIG. 9D). The surprising synergistic effect of combined treatment of PD1-IL2v and SQZ-PBMC is most strikingly seen in the tumor. According to FIG. 9B, combined treatment resulted in 3× more CD8+ cells in tumors (FIG. 9A) and 3.1× more E7-specific T-cells (FIG. 9B) compared to SQZ-PBMC treatment alone. The increased amount of T cells was also seen in the spleen, where combined therapy resulted in 3.2× more CD8+ cells (FIG. 9C) and 2.1× more E7-specific T-cells (FIG. 9D) compared to SQZ-PBMC treatment alone. This suggests that the combination therapy improved the targeted recruitment of immune cells, specifically E7-tetramer-specific CD8+ T cells, within and/or to tumors. The increase in immune cells was reflected by a systemic increase in overall immune cell number, as measured in the spleen within the same mice.

### Example 4. Effects of Treatment on Regulatory T Cells (Tregs) and NK Cells of the Innate Immune Response

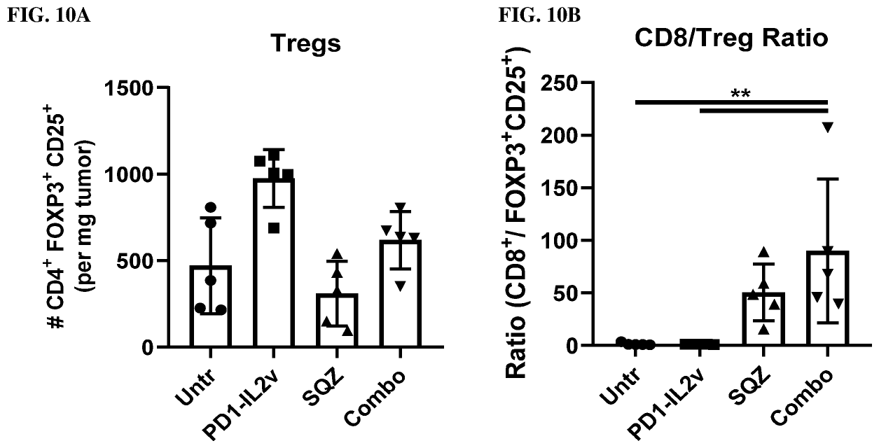

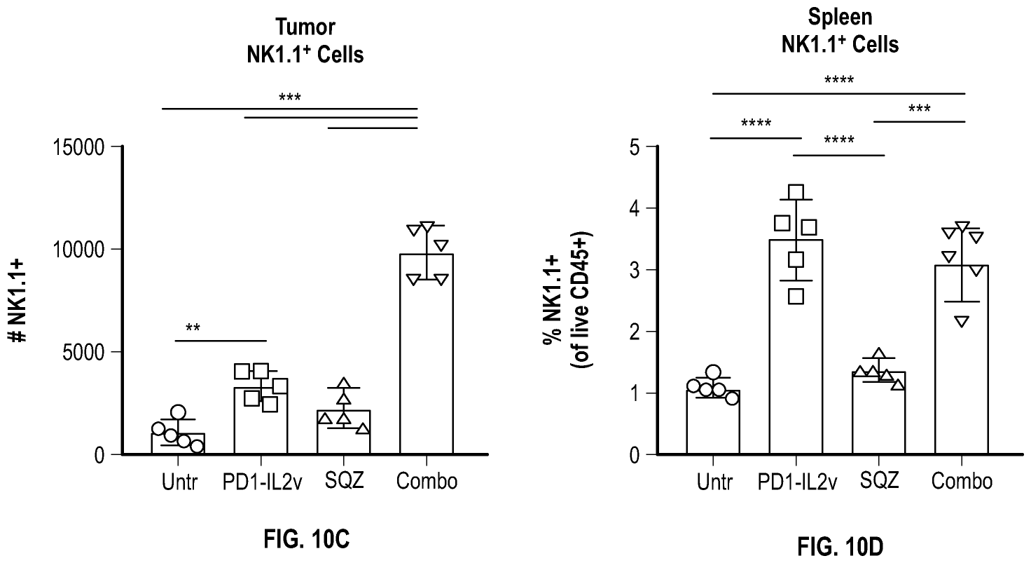

Regulatory T cells co-express CD4, FOXP3 and CD25 proteins which help support the function of these T cells. The number of immuno-positive markers present within the tumor was measured by quantifying CD4, FOXP3 and CD25+ cells across untreated, PD1-IL2v monotherapy, SQZ-PBMC monotherapy, and PD1-IL2v with SQZ-PBMC combined therapy. Measurements were conducted on day 24 or 28 following tumor implantation, and immunofluorescence expression was quantified using flow cytometry. There were no significant differences between the total amounts of immune-positive markers across all treatment groups tested (FIG. 10A). In contrast, when the ratio of CD8+ cells relative to the combined expression of FOXP3+ and CD25+ cells was quantified, it was found that combined treatment resulted in a significant increase in the immune cell ratio compared to untreated and PD1-IL2v monotherapy conditions. This suggests that, in the combination therapy group, there is a heightened amount of CD8+ cytotoxic T cells recruited to actively target the HPV tumors whereas T-regulatory cells were functioning normally to maintain the overall immune system of the mice (FIG. 10B). Tregs are expanded within the tumor of both PD1-IL2v treated groups, and expansion of infiltrating CD8+ cells is greater than that of Tregs in SQZ-PBMC+PD1-IL2v combination group.

Next, the total number of NK1.1+ cells was quantified in tumors across all treatment groups. According to FIG. 10C, PD1-IL2v monotherapy resulted in a significant increase in NK cells in tumors compared to untreated mice. In contrast, SQZ-PBMC monotherapy did not have any effects on the total number of NK cells within the tumors. The total number of NK cells was further increased with the combined therapy of PD1-IL2v and SQZ-PBMC. Similarly, the percentage of NK1.1+ cells of CD45+ was significantly increased by PD1-IL2v monotherapy and PD1-IL2v with SQZ-PBMC combined therapy in the spleen (FIG. 10D). Overall, SQZ-PBMC and PD1-IL2v combination therapy leads to significant increases of tumor-infiltrating NK cells; however, the increase in NK1.1+ cells by combined therapy is similar to PD1-IL2v monotherapy in the spleen.

### Example 5. Combination of Peptide Vaccine and Immunoconjugate

To evaluate the therapeutic efficacy of PD1-IL2v in combination with a peptide vaccine approach, TC1 tumor bearing mice were treated with a peptide vaccine consisting of the E7 long peptide (E7-LP) and CpG, either alone or in combination with the PD1-IL2v bispecific molecule. TC1 cells (kindly provided by Prof TC Wu, Johns Hopkins University, Baltimore, Md.), were implanted subcutaneously on the flank of C57BL/6NCrl mice (purchased from Charles River) at a concentration of 5×10⁵ cells in 100 μl HBBS. Tumor growth was measured with a caliper using the formula V=W2Lπ/6. Animal experiments were conducted under the approved license VD3142.

CpG-B 1826 oligonucleotide (5′-TCCATGAGCTTCCTGACGTT-3′ as phosphorothioated DNA bases; SEQ ID NO:58) was purchased from Microsynth. HPV16 E7 long peptide GQAEPDRAHYNIVTFCCKCDSTLRLCVQSTHVDIR (E7-LP, amino acid 43-77, purity >90%; SEQ ID NO:56) was purchased from Proimmune.

Tumor bearing mice were randomized into the different groups based on the tumor volume. The average starting tumor volume on day 11 post TC1 cell implantation was 180 mm³. Mice were immunized with 15 μg E7-LP and 20 μg CpG. Immunizations of mice were performed subcutaneously in the four limbs. PD1-IL2v was administered i.p. on day 11 at 1 mg/kg, once a week for a duration of four weeks.

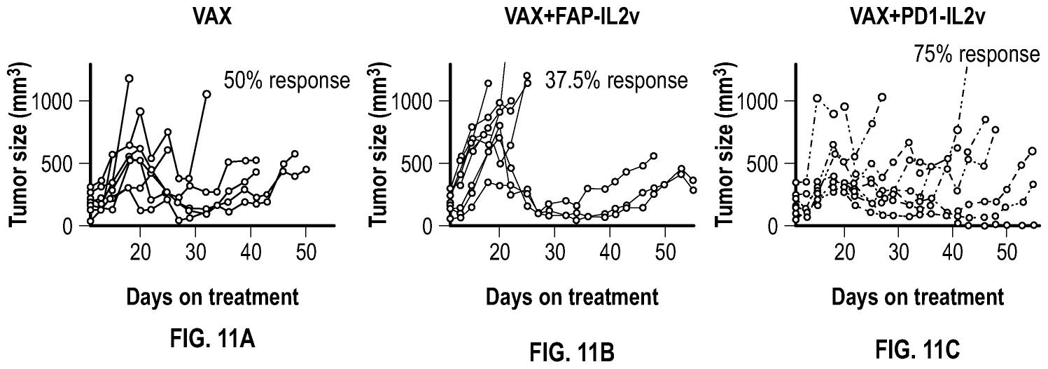

Analysis of the tumor volume revealed that the addition of PD1-IL2v enhanced the efficacy of the E7 vaccine (VAX, FIGS. 11A, 11B). The response to the treatment was estimated by calculating the relative tumor volume and a relative tumor volume smaller than two on day 29 post TC1 cell implantation was considered as a response. By using this criterium 50% of the VAX only mice responded to the treatment, whereas in the VAX+PD1-IL2v treated mice the response rate was 75%. Long time survival analysis showed a modest increase in the survival of mice in the VAX+PD1-IL2v treatment group, compared to the VAX only group (FIG. 11).

### Example 6. Administration of PD1-IL2v after SQZ-PBMC Immunization Boosts Antigen Specific CD8+ T Cell Response

C57BL6 mice (7-weeks old) were purchased from The Jackson Laboratories. Spleens from donor mice were harvested and processed into a single cell suspension. The B cells were depleted and Ovalbumin (OVA) was delivered by using the SQZ Biotechnologies Cell Squeeze® technology. The squeezed splenocytes were conditioned for 4 hours in CpG (1 μM), resuspended in PBS, and administered retro-orbitally in recipient C57BL6 mice. PD1-IL2v were administered as a single dose at 1.5 mg/kg retro-orbitally on the appropriate day as per Table 3.

TABLE 3
Treatment groups and mouse numbers

| | Day of PD1-IL2v | |
|---|---|---|
| SQZ Immunization | administration | Number |
| (5 × 10⁶ cells/mouse) | (1.5 mg/kg) | of mice |
| SQZ-PBMC | None | 5 |
| SQZ-PBMC-OVA | None | 5 |
| SQZ-PBMC-OVA | Day 2 | 5 |
| SQZ-PBMC-OVA | Day 3 | 5 |
| SQZ-PBMC-OVA | Day 4 | 4 |

Fourteen days after immunization, mice were euthanized, spleens isolated, processed into a cell suspension and re-stimulated ex-vivo with OVA peptide (SIINFEKL, 1 μg/mL) in the presence of α-CD28 (2 μg/mL) followed by IFN-γ intracellular cytokine staining. IFN-γ production in this assay is used as a proxy for OVA antigen (OVA) specific CD8+ T cells.

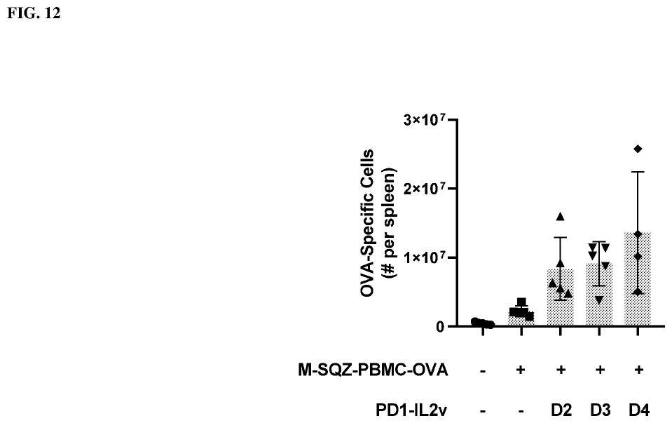

The combination of SQZ-PBMC-OVA and PD1-IL2v leads to an increase in the total number of CD8+ cells expressing IFN-γ (FIG. 12). This suggests that PD1-IL2v expands antigen specific CD8+ T cells after SQZ-PBMC immunization.

SEQUENCES

| | | SEQ ID |
|---|---|---|
| | | NO |
| | Amino Acid Sequence | |
| | N-terminal to C-terminal | |
| PD-1 minimal | SSYT | 1 |
| HVR-H1 | | |
| PD-1 minimal | SGGGRDIY | 2 |
| HVR-H2 | | |
| PD-1 minimal | GRVYF | 3 |
| HVR-H3 | | |
| PD-1 minimal | TSDNSF | 4 |
| HVR-L1 | | |
| PD-1 minimal | RSSTLES | 5 |
| HVR-L2 | | |
| PD-1 minimal | NYDVPW | 6 |
| HVR-L3 | | |
| fragment of | RDN | 7 |
| FR-H3 (RDN | | |
| at Kabat pos. | | |
| 71-73) | | |
| PD-1 HVR-H1 | GFSFSSY | 8 |
| PD-1 HVR-H2 | GGR | 9 |
| PD-1 HVR-H3 | TGRVYFALD | 10 |
| PD-1 HVR-L1 | SESVDTSDNSF | 11 |
| PD-1 HVR-L2 | RSS | 12 |
| PD-1 HVR-L3 | NYDVPW | 13 |
| PD-1 VH | EVQLLESGGGLVQPGGSLRLSCAASGFSFSSYTMSWVRQAPGKGLEWVATIS | 14 |
| (1, 2, 3, 4) | GGGRDIYYPDSVKGRFTISRDNSKNTLYLQMNSLRAEDTAVYYCVLLTGRVY | |
| | FALDSWGQGTLVTVSS | |
| PD-1 VL(1) | DIVMTQSPDSLAVSLGERATINCKASESVDTSDNSFIHWYQQKPGQSPKLLI | 15 |
| | YRSSTLESGVPDRFSGSGSGTDFTLTISSLQAEDVAVYYCQQNYDVPWTFGQ | |
| | GTKVEIK | |
| PD-1 VL (2) | DVVMTQSPLSLPVTLGQPASISCRASESVDTSDNSFIHWYQQRPGQSPRLLI | 16 |
| | YRSSTLESGVPDRFSGSGSGTDFTLKISRVEAEDVGVYYCQQNYDVPWTFGQ | |
| | GTKVEIK | |
| PD-1 VL (3) | EIVLTQSPATLSLSPGERATLSCRASESVDTSDNSFIHWYQQKPGQSPRLLI | 17 |
| | YRSSTLESGIPARFSGSGSGTDFTLTISSLEPEDFAVYYCQQNYDVPWTFGQ | |
| | GTKVEIK | |
| PD-1 VL (4) | EIVLTQSPATLSLSPGERATLSCRASESVDTSDNSFIHWYQQKPGQSPRLLI | 18 |
| | YRSSTLESGIPARFSGSGSGTDFTLTISSLEPEDFAVYYCQQNYDVPWTFGQ | |
| | GTKVEIK | |
| Human IL-2 | APTSSSTKKTQLQLEHLLLDLQMILNGINNYKNPKLTRMLTFKFYMPKKATE | 19 |
| | LKHLQCLEEELKPLEEVLNLAQSKNFHLRPRDLISNINVIVLELKGSETTFM | |
| | CEYADETATIVEFLNRWITFCQSIISTLT | |
| Human IL-2 | APASSSTKKTQLQLEHLLLDLQMILNGINNYKNPKLTRMLTAKFAMPKKATE | 20 |
| (T3A, F42A, | LKHLQCLEEELKPLEEVLNGAQSKNFHLRPRDLISNINVIVLELKGSETTFM | |
| Y45Y, L72G, | CEYADETATIVEFLNRWITFAQSIISTLT | |
| C125A) | | |
| linker | GGGGSGGGGSGGGGS | 21 |
| PD-1 IL2v- | EVQLLESGGGLVQPGGSLRLSCAASGFSFSSYTMSWVRQAPGKGLEWVATIS | 22 |
| HC with IL2v | GGGRDIYYPDSVKGRFTISRDNSKNTLYLQMNSLRAEDTAVYYCVLLTGRVY | |
| (Fc knob, | FALDSWGQGTLVTVSSASTKGPSVFPLAPSSKSTSGGTAALGCLVKDYFPEP | |
| LALAPG) | VTVSWNSGALTSGVHTFPAVLQSSGLYSLSSVVTVPSSSLGTQTYICNVNHK | |
| | PSNTKVDKKVEPKSCDKTHTCPPCPAPEAAGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALGAPIEKTISKAKGQPREPQVYTLPPCRDELTKN | |
| | QVSLWCLVKGFYPSDIAVEWESNGQPENNYKTTPPVLDSDGSFFLYSKLTVD | |
| | KSRWQQGNVFSCSVMHEALHNHYTQKSLSLSPGGGGGSGGGGSGGGGSAPAS | |
| | SSTKKTQLQLEHLLLDLQMILNGINNYKNPKLTRMLTAKFAMPKKATELKHL | |
| | QCLEEELKPLEEVLNGAQSKNFHLRPRDLISNINVIVLELKGSETTFMCEYA | |
| | DETATIVEFLNRWITFAQSIISTLT | |
| PD-1 IL2v- | EVQLLESGGGLVQPGGSLRLSCAASGFSFSSYTMSWVRQAPGKGLEWVATIS | 23 |
| HC without | GGGRDIYYPDSVKGRFTISRDNSKNTLYLQMNSLRAEDTAVYYCVLLTGRVY | |
| IL2v (Fc hole, | FALDSWGQGTLVTVSSASTKGPSVFPLAPSSKSTSGGTAALGCLVKDYFPEP | |
| LALAPG) | VTVSWNSGALTSGVHTFPAVLQSSGLYSLSSVVTVPSSSLGTQTYICNVNHK | |
| | PSNTKVDKKVEPKSCDKTHTCPPCPAPEAAGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALGAPIEKTISKAKGQPREPQVCTLPPSRDELTKN | |
| | QVSLSCAVKGFYPSDIAVEWESNGQPENNYKTTPPVLDSDGSFFLVSKLTVD | |
| | KSRWQQGNVFSCSVMHEALHNHYTQKSLSLSP | |
| PD-1 IL2v- | EVQLLESGGGLVQPGGSLRLSCAASGFSFSSYTMSWVRQAPGKGLEWVATIS | 24 |
| HC without | GGGRDIYYPDSVKGRFTISRDNSKNTLYLQMNSLRAEDTAVYYCVLLTGRVY | |
| IL2v (Fc hole, | FALDSWGQGTLVTVSSASTKGPSVFPLAPSSKSTSGGTAALGCLVKDYFPEP | |
| LALAPG, | VTVSWNSGALTSGVHTFPAVLQSSGLYSLSSVVTVPSSSLGTQTYICNVNHK | |
| HYRF) | PSNTKVDKKVEPKSCDKTHTCPPCPAPEAAGGPSVFLFPPKPKDTLMISRTP | |
| | EVTCVVVDVSHEDPEVKFNWYVDGVEVHNAKTKPREEQYNSTYRVVSVLTVL | |
| | HQDWLNGKEYKCKVSNKALGAPIEKTISKAKGQPREPQVCTLPPSRDELTKN | |
| | QVSLSCAVKGFYPSDIAVEWESNGQPENNYKTTPPVLDSDGSFFLVSKLTVD | |
| | KSRWQQGNVFSCSVMHEALHNRFTQKSLSLSP | |
| PD-1 IL2v- | DIVMTQSPDSLAVSLGERATINCKASESVDTSDNSFIHWYQQKPGQSPKLLI | 25 |
| LC | YRSSTLESGVPDRFSGSGSGTDFTLTISSLQAEDVAVYYCQQNYDVPWTFGQ | |
| | GTKVEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSP | |
| | VTKSFNRGEC | |
| Human IL-2 | APTSSSTKKTQLQLEHLLLDLQMILNGINNYKNPKLTRMLTFKFYMPKKATE | 26 |
| (C125A) | LKHLQCLEEELKPLEEVLNLAQSKNFHLRPRDLISNINVIVLELKGSETTFM | |
| | CEYADETATIVEFLNRWITFCQSIISTLTAPASSSTKKTQLQLEHLLLDLQM | |
| | ILNGINNYKNPKLTRMLTAKFAMPKKATELKHLQCLEEELKPLEEVLNGAQS | |
| | KNFHLRPRDLISNINVIVLELKGSETTFMCEYADETATIVEFLNRWITFAQS | |
| | IISTLT | |
| Human IL-2 | APASSSTKKTQLQLEHLLLDLQMILNGINNYKNPKLTRMLTAKFAMPKKATE | 27 |
| T3A/F42A/ | LKHLQCLEEELKPLEEVLNGAQSKNFHLRPRDLISNINVIVLELKGSETTFM | |
| Y45A/L72G | CEYADETATIVEFLNRWITFAQSIISTLT | |
| (C125A) | | |
| FAP | EIVLTQSPGTLSLSPGERATLSCRASQSVTSSYLAWYQQKPGQAPRLLINVG | 28 |
| 3F2VL | SRRATGIPDRFSGSGSGTDFTLTISRLEPEDFAVYYCQQGIMLPPTFGQGTK | |
| | VEIK | |
| FAP | EVQLLESGGGLVQPGGSLRLSCAASGFTFSSYAMSWVRQAPGKGLEWVSAIS | 29 |
| 3F2VH | GSGGSTYYADSVKGRFTISRDNSKNTLYLQMNSLRAEDTAVYYCAKGWFGGF | |
| | NYWGQGTLVTVSS | |
| FAP | EIVLTQSPGTLSLSPGERATLSCRASQSVSRSYLAWYQQKPGQAPRLLIIGA | 30 |
| 4G8 VL | STRATGIPDRFSGSGSGTDFTLTISRLEPEDFAVYYCQQGQVIPPTFGQGTK | |
| | VEIK | |
| FAP | EVQLLESGGGLVQPGGSLRLSCAASGFTFSSYAMSWVRQAPGKGLEWVSAIS | 31 |
| 4G8 VH | GSGGSTYYADSVKGRFTISRDNSKNTLYLQMNSLRAEDTAVYYCAKGWLGNF | |
| | DYWGQGTLVTVSS | |
| FAP | EIVLTQSPGTLSLSPGERATLSCRASQSVTSSYLAWYQQKPGQAPRLLINVG | 32 |
| 4B9 VL | SRRATGIPDRFSGSGSGTDFTLTISRLEPEDFAVYYCQQGIMLPPTFGQGTK | |
| | VEIK | |
| FAP | EVQLLESGGGLVQPGGSLRLSCAASGFTFSSYAMSWVRQAPGKGLEWVSAII | 33 |
| 4B9 VH | GSGASTYYADSVKGRFTISRDNSKNTLYLQMNSLRAEDTAVYYCAKGWFGGF | |
| | NYWGQGTLVTVSS | |
| FAP | EIVLTQSPGTLSLSPGERATLSCRASQSVSRSYLAWYQQKPGQAPRLLIIGA | 34 |
| 28H1 VL | STRATGIPDRFSGSGSGTDFTLTISRLEPEDFAVYYCQQGQVIPPTFGQGTK | |
| | VEIK | |
| FAP | EVQLLESGGGLVQPGGSLRLSCAASGFTFSSHAMSWVRQAPGKGLEWVSAIW | 35 |
| 28H1 VH | ASGEQYYADSVKGRFTISRDNSKNTLYLQMNSLRAEDTAVYYCAKGWLGNFD | |
| | YWGQGTLVTVSS | |
| FAP | EVQLLESGGGLVQPGGSLRLSCAASGFTFSSYAMSWVRQAPGKGLEWVSAII | 36 |
| 29B11 VL | GSGGITYYADSVKGRFTISRDNSKNTLYLQMNSLRAEDTAVYYCAKGWFGGF | |
| | NYWGQGTLVTVSS | |
| FAP | EVQLLESGGGLVQPGGSLRLSCAASGFTFSSYAMSWVRQAPGKGLEWVSAII | 37 |
| 29B11 VH | GSGGITYYADSVKGRFTISRDNSKNTLYLQMNSLRAEDTAVYYCAKGWFGGF | |
| | NYWGQGTLVTVSS | |
| FAP | EVQLLESGGGLVQPGGSLRLSCAASGFTFSSYAMSWVRQAPGKGLEWVSAII | 38 |
| 4B9 HC fused | GSGASTYYADSVKGRFTISRDNSKNTLYLQMNSLRAEDTAVYYCAKGWFGGF | |
| to IL2v | NYWGQGTLVTVSSASTKGPSVFPLAPSSKSTSGGTAALGCLVKDYFPEPVTV | |
| (knob) | SWNSGALTSGVHTFPAVLQSSGLYSLSSWTVPSSSLGTQTYICNVNHKPSN | |
| | TKVDKKVEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALGAPIEKTISKAKGQPREPQVYTLPPCRDELTKNQVS | |
| | LWCLVKGFYPSDIAVEWESNGQPENNYKTTPPVLDSDGSFFLYSKLTVDKSR | |
| | WQQGNVFSCSVMHEALHNHYTQKSLSLSPGGGGGSGGGGSGGGGSAPASSST | |
| | KKTQLQLEHLLLDLQMILNGINNYKNPKLTRMLTAKFAMPKKATELKHLQCL | |
| | EEELKPLEEVLNGAQSKNFHLRPRDLISNINVIVLELKGSETTFMCEYADET | |
| | ATIVEFLNRWITFAQSIISTLT | |
| FAP | EVQLLESGGGLVQPGGSLRLSCAASGFTFSSYAMSWVRQAPGKGLEWVSAII | 39 |
| 4B9 HC | GSGASTYYADSVKGRFTISRDNSKNTLYLQMNSLRAEDTAVYYCAKGWFGGF | |
| (hole) | NYWGQGTLVTVSSASTKGPSVFPLAPSSKSTSGGTAALGCLVKDYFPEPVTV | |
| | SWNSGALTSGVHTFPAVLQSSGLYSLSSWTVPSSSLGTQTYICNVNHKPSN | |
| | TKVDKKVEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALGAPIEKTISKAKGQPREPQVCTLPPSRDELTKNQVS | |
| | LSCAVKGFYPSDIAVEWESNGQPENNYKTTPPVLDSDGSFFLVSKLTVDKSR | |
| | WQQGNVFSCSVMHEALHNHYTQKSLSLSPGK | |
| FAP | EIVLTQSPGTLSLSPGERATLSCRASQSVTSSYLAWYQQKPGQAPRLLINVG | 40 |
| 3F2LC | SRRATGIPDRFSGSGSGTDFTLTISRLEPEDFAVYYCQQGIMLPPTFGQGTK | |
| | VEIKRTVAAPSVFIFPPSDEQLKSGTASVVCLLNNFYPREAKVQWKVDNALQ | |
| | SGNSQESVTEQDSKDSTYSLSSTTLSKADYEKH | |
| | KVYACEVTHQGLSSPVTKSFNRGEC | |
| FAP | EIVLTQSPGTLSLSPGERATLSCRASQSVSRSYLAWYQQKPGQAPRLLIIGA | 41 |
| 4G8LC | STRATGIPDRFSGSGSGTDFTLTISRLEPEDFAVYYCQQGQVIPPTFGQGTK | |
| | VEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTK | |
| | SFNRGEC | |
| FAP | EVQLLESGGGLVQPGGSLRLSCAASGFTFSSHAMSWVRQAPGKGLEWVSAIW | 42 |
| HC fused to | ASGEQYYADSVKGRFTISRDNSKNTLYLQMNSLRAEDTAVYYCAKGWLGNFD | |
| IL-2 | YWGQGTLVTVSSASTKGPSVFPLAPSSKSTSGGTAALGCLVKDYFPEPVTVS | |
| (knob) | WNSGALTSGVHTFPAVLQSSGLYSLSSVVTVPSSSLGTQTYICNVNHKPSNT | |
| | KVDKKVEPKSCDKTHTCPPCPAPEAAGGPSVFLFPPKPKDTLMISRTPEVTC | |
| | VWDVSHEDPEVKFNWYVDGVEVHNAKTKPREEQYNSTYRVVSVLTVLHQDW | |
| | LNGKEYKCKVSNKALGAPIEKTISKAKGQPREPQVYTLPPCRDELTKNQVSL | |
| | WCLVKGFYPSDIAVEWESNGQPENNYKTTPPVLDSDGSFFLYSKLTVDKSRW | |
| | QQGNVFSCSVMHEALHNHYTQKSLSLSPGGGGGSGGGGSGGGGSAPASSSTK | |
| | KTQLQLEHLLLDLQMILNGINNYKNPKLTRMLTAKFAMPKKATELKHLQCLE | |
| | EELKPLEEVLNGAQSKNFHLRPRDLISNINVIVLELKGSETTFMCEYADETA | |
| | TIVEFLNRWITFAQSIISTLT | |
| FAP | EVQLLESGGGLVQPGGSLRLSCAASGFTFSSHAMSWVRQAPGKGLEWVSAIW | 43 |
| 28H1 HC | ASGEQYYADSVKGRFTISRDNSKNTLYLQMNSLRAEDTAVYYCAKGWLGNFD | |
| (hole) | YWGQGTLVTVSSASTKGPSVFPLAPSSKSTSGGTAALGCLVKDYFPEPVTVS | |
| | WNSGALTSGVHTFPAVLQSSGLYSLSSVVTVPSSSLGTQTYICNVNHKPSNT | |
| | KVDKKVEPKSCDKTHTCPPCPAPEAAGGPSVFLFPPKPKDTLMISRTPEVTC | |
| | VWDVSHEDPEVKFNWYVDGVEVHNAKTKPREEQYNSTYRVVSVLTVLHQDW | |
| | LNGKEYKCKVSNKALGAPIEKTISKAKGQPREPQVCTLPPSRDELTKNQVSL | |
| | SCAVKGFYPSDIAVEWESNGQPENNYKTTPPVLDSDGSFFLVSKLTVDKSRW | |
| | QQGNVFSCSVMHEALHNHYTQKSLSLSPGK | |
| FAP | EVQLLESGGGLVQPGGSLRLSCAASGFTFSSYAMSWVRQAPGKGLEWVSAIS | 44 |
| 4G8HC | GSGGSTYYADSVKGRFTISRDNSKNTLYLQMNSLRAEDTAVYYCAKGWLGNF | |
| (knob) | DYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALGAPIEKTISKAKGQPREPQVYTLPPCRDELTKNQVS | |
| | LWCLVKGFYPSDIAVEWESNGQPENNYKTTPPVLDSDGSFFLYSKLTVDKSR | |
| | WQQGNVFSCSVMHEALHNHYTQKSLSLSPGGGGGSGGGGSGGGGSAPASSST | |
| | KKTQLQLEHLLLDLQMILNGINNYKNPKLTRMLTAKFAMPKKATELKHLQCL | |
| | EEELKPLEEVLNGAQSKNFHLRPRDLISNINVIVLELKGSETTFMCEYADET | |
| | ATIVEFLNRWITFAQSIISTLT | |
| FAP | EVQLLESGGGLVQPGGSLRLSCAASGFTFSSYAMSWVRQAPGKGLEWVSAIS | 45 |
| 4G8HC | GSGGSTYYADSVKGRFTISRDNSKNTLYLQMNSLRAEDTAVYYCAKGWLGNF | |
| (hole) | DYWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSN | |
| | TKVDKKVEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQD | |
| | WLNGKEYKCKVSNKALGAPIEKTISKAKGQPREPQVCTLPPSRDELTKNQVS | |
| | LSCAVKGFYPSDIAVEWESNGQPENNYKTTPPVLDSDGSFFLVSKLTVDKSR | |
| | WQQGNVFSCSVMHEALHNHYTQKSLSLSPGK | |
| HPV16- | TIHDIILECV | 46 |
| E6(29-38), | | |
| human epitope | | |
| HPV16- | EVYDFAFRDL | 47 |
| E6(48-57), | | |
| murine epitope | | |
| HPV16- | YMLDLQPETT | 48 |
| E7(ll-20), | | |
| human epitope | | |
| HPV16- | RAHYNIVTF | 49 |
| E7(49-57), | | |
| murine epitope | | |
| HPV16- | LPQLSTELQTTIHDIILECVYSKQQLLRREVYDFAF | 50 |
| E6(19-54) | | |
| SLP, human | | |
| HPV16- | QLCTELQTTIHDIILECVYCKQQLL | 51 |
| E6(21-45) | | |
| SLP, human | | |
| HPV16- | KQQLLRREVYDFAFRDLCIVYRDGN | 52 |
| E6(41-65) | | |
| SLP, native | | |
| murine | | |
| HPV16- | VYSKQQLLRREVYDFAFRDLSIVYRDGNPYAVSDK | 53 |
| E6(38-72) | | |
| SLP, classic | | |
| murine | | |
| HPV16-E7(1- | MHGDTPTLHEYMLDLQPETTDLYCYEQLNDSSEEE | 54 |
| 35) SLP, | | |
| human | | |
| HPV16-E7.6 | QLCTELQTYMLDLQPETTYCKQQLL | 55 |
| SLP, human | | |
| HPV16- | GQAEPDRAHYNIVTFCCKCDSTLRLCVQSTHVDIR | 56 |
| E7(43-77) | | |
| SLP, native | | |
| murine | | |
| HPV16- | GQAEPDRAHYNIVTFSSKSDSTLRLSVQSTHVDIR | 57 |
| E7(43-77) | | |
| SLP, classic | | |
| murine | | |
| hIL-2 signal | MYRMQLLSCIALSLALVTNS | 59 |
| peptide | | |
| HPV16- | LPQLSTELQT | 60 |
| E6(19-28) N- | | |
| terminal | | |
| polypeptide, | | |
| human | | |
| HPV16- | QLCTELQT | 61 |
| E6(21-28) N- | | |
| terminal | | |
| polypeptide, | | |
| human | | |
| HPV16- | KQQLLRR | 62 |
| E6(41-47) N- | | |
| terminal | | |
| polypeptide, | | |
| native murine | | |
| HPV16- | VYSKQQLLRR | 63 |
| E6(38-47) N- | | |
| terminal | | |
| polypeptide, | | |
| classic murine | | |
| HPV16-E7(1- | MHGDTPTLHE | 64 |
| 10) N-terminal | | |
| polypeptide, | | |
| human | | |
| HPV16- | GQAEPD | 65 |
| E7(43-48) N- | | |
| terminal | | |
| polypeptide, | | |
| murine | | |
| HPV16- | YSKQQLLRREVYDFAF | 66 |
| E6(39-54) C- | | |
| terminal | | |
| polypeptide, | | |
| human | | |
| HPV16- | YCKQQLL | 67 |
| E6(39-45) C- | | |
| terminal | | |
| polypeptide, | | |
| human | | |
| HPV16- | CIVYRDGN | 68 |
| E6(58-65) c- | | |
| terminal | | |
| polypeptide, | | |
| native murine | | |
| HPV16- | SIVYRDGNPYAVSDK | 69 |
| E6(58-72) C- | | |
| terminal | | |
| polypeptide, | | |
| classic murine | | |
| HPV16- | DLYCYEQLNDSSEEE | 70 |
| E7(21-35) C- | | |
| terminal | | |
| polypeptide, | | |
| human | | |
| HPV16- | CCKCDSTLRLCVQSTHVDIR | 71 |
| E7(58-77 C- | | |
| terminal | | |
| polypeptide, | | |
| native murine | | |
| HPV16- | SSKSDSTLRLSVQSTHVDIR | 72 |
| E7(58-77) C- | | |
| terminal | | |
| polypeptide, | | |
| classic murine | | |
| | Nucleic Acid Sequence (5′ to 3′) | |
| CpG-B 1826 | TCCATGAGCTTCCTGACGTT as phosphorothioated DNA bases | 58 |
| CpG ODN | TCGTCGTTTTGTCGTTTTGTCGTT | 73 |
| 2006 (also | | |
| known as CpG | | |
| 7909) | | |

What is claimed is:

1. A method for stimulating an immune response in an individual, the method comprising
a) administering an effective amount of a composition comprising nucleated cells to an individual, wherein the nucleated cells comprise an exogenous antigen; and
b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

2. A method for stimulating an immune response to a tumor antigen in an individual, the method comprising
a) administering an effective amount of a composition comprising nucleated cells to an individual, wherein the nucleated cells comprise an exogenous tumor antigen; and
b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

3. A method for enhancing a nucleated cell-based immunotherapy, the method comprising administering an effective amount of an immunoconjugate in conjunction with the nucleated cell-based immunotherapy, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

4. A method for treating a disease in an individual, the method comprising
a) administering an effective amount of a composition comprising nucleated cells to an individual, wherein the nucleated cells comprise an exogenous antigen associated with the disease; and
b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

5. A method of vaccinating an individual in need thereof, the method comprising
a) administering an effective amount of a composition comprising nucleated cells to an individual, wherein the nucleated cells comprise an exogenous antigen; and
b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

6. The method of claim 5, wherein the individual has a disease responsive to vaccination.

7. The method of any one of claims 4-6, wherein the disease is cancer, an infectious disease, or a viral-associated disease.

8. A method for reducing tumor growth in an individual, the method comprising
a) administering an effective amount of a composition comprising nucleated cells to an individual, wherein the nucleated cells comprise an exogenous tumor antigen; and
b) administering an effective amount of an immunoconjugate to the individual, wherein the immunoconjugate comprises a mutant IL-2 polypeptide and a second polypeptide, wherein the mutant IL-2 polypeptide is a human IL-2 molecule comprising the amino acid substitutions F42A, Y45A and L72G (numbering relative to the human IL-2 sequence SEQ ID NO:19), and wherein the second polypeptide is capable of specific binding to a T cell, a tumor cell, or the tumor cell environment.

9. The method of any one of claims 1-8, wherein the second polypeptide binds a T cell.

10. The method of claim 11, wherein the second polypeptide binds PD-1 expressed on the T cell.

11. The method of claim 10, wherein the second polypeptide is an antigen-binding moiety that specifically binds PD-1.

12. The method of claim 11, wherein the anti-PD-1 antigen-binding moiety comprises
(a) a heavy chain variable region (VH) comprising a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6; or
(b) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO:13.

13. The method of claim 11 or 12, wherein the anti-PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO: 14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO:17, and SEQ ID NO:18.

14. The method of any one of claims 11-13, wherein the anti-PD-1 antigen-binding moiety comprises (a) a heavy chain variable region (VH) comprising an amino acid sequence of SEQ ID NO:14, and (b) a light chain variable region (VL) comprising an amino acid sequence of SEQ ID NO:15, SEQ ID NO:16, SEQ ID NO:17, and SEQ ID NO:18.

15. The method of any one of claims 11-14, wherein the anti-PD-1 antigen-binding moiety comprises (a) a heavy chain variable region (VH) comprising an amino acid sequence of SEQ ID NO:14, and (b) a light chain variable region (VL) comprising an amino acid sequence of SEQ ID NO:15.

16. The method of any one of claims 11-15, wherein the immunoconjugate comprises a polypeptide comprising an amino acid sequence that is at least about 80%, 85%, 90%, 95%, 96%, 97%, 98%, 99% or 100% identical to the sequence of SEQ ID NO:22, a polypeptide comprising an amino acid sequence that is at least about 80%, 85%, 90%, 95%, 96%, 97%, 98%, 99% or 100% identical to the sequence of SEQ ID NO:24, and a polypeptide comprising an amino acid sequence that is at least about 80%, 85%, 90%, 95%, 96%, 97%, 98%, 99% or 100% identical to the sequence of SEQ ID NO:25.

17. The method of any one of claims 11-16, wherein the immunoconjugate comprises a polypeptide sequence of SEQ ID NO: 22, a polypeptide sequence of SEQ ID NO:24, and two polypeptide sequences of SEQ ID NO:25.

18. The method of any one of claims 1-8, wherein the second polypeptide specifically binds a target antigen presented on a tumor cell or in a tumor cell environment.

19. The method of claim 18, wherein the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP).

20. The method of any one of claims 1-8 and 18-19, wherein the second polypeptide binds FAP.

21. The method of claim 19 of 20, wherein the second polypeptide is an antigen-binding moiety that specifically binds FAP.

22. The method of claim 21, wherein the antigen-binding moiety the specifically bind FAP comprising (i) the heavy chain variable region sequence of SEQ ID NO:29 and the light chain variable region sequence of SEQ ID NO: 28; (ii) the heavy chain variable region sequence of SEQ ID NO:31 and the light chain variable region sequence of SEQ ID NO:30; (iii) the heavy chain variable region sequence of SEQ ID NO: 33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36.

23. The method of claim 21 or 22, wherein the antigen-binding moiety the specifically bind FAP comprising (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO: 231; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41.

24. The method of any one of claims 21-23, wherein antigen-binding moiety the specifically bind FAP comprises the heavy chain variable region sequence of SEQ ID NO:33 and the light chain variable region sequence of SEQ ID NO:32.

25. The method of any one of claims 21-24, wherein the immunoconjugate comprises a polypeptide sequence of SEQ ID NO:38, a polypeptide sequence of SEQ ID NO:39, and a polypeptide sequence of SEQ ID NO: 40.

26. The method of any one of claims 1-25, wherein the mutant IL-2 polypeptide further comprises the amino acid substitution T3A and/or the amino acid substitution C125A.

27. The method of any one of claims 1-26, wherein the mutant IL-2 polypeptide comprises an amino acid sequence that is at least about 95%, 96%, 97%, 98%, 99% or 100% identical to the amino acid sequence of SEQ ID NO:20, wherein the mutant IL-2 polypeptide shows reduced affinity to the high-affinity IL-2 receptor and substantially similar affinity to the intermediate-affinity IL-2 receptor, each compared to a wild-type IL-2 polypeptide.

28. The immunoconjugate of any one of claims 1-27, wherein the mutant IL-2 polypeptide comprises the amino acid sequence of SEQ ID NO:20.

29. The method of any one of claims 1-28, wherein the nucleated cells are immune cells.

30. The method of any one of claims 1-29, wherein the nucleated cells are a plurality of peripheral blood mononuclear cells (PBMCs).

31. The method of claim 30, wherein the plurality of PBMCs comprise two or more of T cell, B cell, NK cell, monocytes, dendritic cells or NK-T cells.

32. The method of any one of claims 1-31, wherein the nucleated cells are one or more of T cells, B cells, NK cells, monocytes, dendritic cells and/or NK-T cells.

33. The method of any one of claims 1-32, wherein the exogenous antigen is delivered to the nucleated cells intracellularly.

34. The method of any one of claims 1-33, wherein the exogenous antigen is a disease-associated antigen.

35. The method of claim 34, wherein the disease-associated antigen is a cancer antigen, an infectious disease antigen, or a viral-associated disease antigen.

36. The method of any one of claims 1-35, wherein the exogenous antigen is a human papillomavirus (HPV) antigen.

37. The method of any one of claims 1-36, wherein the composition further comprises an adjuvant.

38. The method of claim 37, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

39. The method of any one of claims 1-38, wherein the nucleated cells comprising the exogenous antigen are prepared by a process comprising the steps of:
a) passing a cell suspension comprising input nucleated cells through a cell-deforming constriction, wherein a diameter of the constriction is a function of a diameter of the input nucleated cells in the suspension, thereby causing perturbations of the input nucleated cells large enough for the exogenous antigen to pass through to form perturbed input nucleated cells;
b) incubating the perturbed input nucleated cells with the exogenous antigen for a sufficient time to allow the exogenous antigen to enter the perturbed input nucleated cells, thereby generating nucleated cells comprising the exogenous antigen.

40. The method of claim 39, wherein the width of the constriction is about 10% to about 99% of the mean diameter of the input nucleated cells.

41. The method of claim 39 or 40, wherein the width of the constriction is about 4.2 μm to about 6 μm.

42. The method of any one of claims 39-41, wherein the width of the constriction is about 4.2 μm to about 4.8 μm.

43. The method of any one of claims 39-42, wherein the width of the constriction is about 4.5 μm.

44. The method of any one of claims 39-43, wherein the cell suspension comprising the plurality of input nucleated cells are passed through multiple constrictions wherein the multiple constrictions are arranged in series and/or in parallel.

45. The method of any one of claims 39-44, wherein the exogenous antigen is present in at least about 70% of the nucleated cells following incubation of the perturbed input nucleated cells with the exogenous antigen.

46. The method of any one of claims 1-45, wherein the nucleated cells are conditioned with an adjuvant to form conditioned cells.

47. The method of claim 46, wherein the nucleated cells are incubated with the adjuvant for about 1 hour to about 24 hours, about 2 hours to about 10 hours, about 3 hours to about 6 hours, or about 4 hours for the cells to condition.

48. The method of claim 46 or 47, wherein the nucleated cells are conditioned before or after introducing the exogenous antigen into the nucleated cells.

49. The method of any one of claims 46-48, wherein the adjuvant is a CpG oligodeoxynucleotide (ODN), LPS, IFN-α, STING agonists, RIG-I agonists, poly I.C, R837, R848, a TLR3 agonist, a TLR4 agonist or a TLR 9 agonist.

50. The method of any one of claims 46-49, wherein the adjuvant is a CpG oligodeoxynucleotide (ODN).

51. The method of any one of claims 46-50, wherein the adjuvant is CpG 7909.

52. The method of any one of claims 46-51, wherein the conditioned cells are a conditioned plurality of modified PBMCs.

53. The method of claim 52, wherein the plurality of modified PBMCs are further modified to increase expression of one or more of co-stimulatory molecules.

54. The method of claim 53, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

55. The method of any one of claims 52-54, wherein the plurality of modified PBMCs are further modified to increase expression of one or more cytokines.

56. The method of claim 55, wherein the cytokine is IL-15, IL-12, IL-2, IFN-α, or IL-21.

57. The method of any one of claim 52-56, wherein one or more co-stimulatory molecules is upregulated in the B cells of the conditioned plurality of modified PBMCs compared to the B cells in the plurality of nonmodified PBMCs, wherein the co-stimulatory molecule is CD80 and/or CD86.

58. The method of any one of claims 52-57, wherein the plurality of modified PBMCs have increased expression of one or more of IFN-γ, IL-6, MCP-1, MIP-1β, IP-10, or TNF-α compared to a plurality of unconditioned PBMCs.

59. The method of claim 58, wherein the expression of one or more of IFN-γ, IL-6, MCP-1, MIP-1β, IP-10, or TNF-α is increased by more than about 1.2-fold, 1.5-fold, 1.8-fold, 2-fold, 3-fold, 4-fold, 5-fold, 8-fold, or more than 10-fold compared to the plurality of unconditioned PBMCs.

60. The method of any one of claims 1-59, wherein the immunoconjugate is administered before, at the same time, or after administration of the composition comprising nucleated cells.

61. The method of any one of claims 1-60, wherein the composition comprising nucleated cells is administered a plurality of times.

62. The method of any one of claims 1-61, wherein the immunoconjugate is administered a plurality of times after administration of the composition comprising nucleated cells.

63. The method of any one of claims 1-62, wherein the composition and/or the immunoconjugate is administered intravenously.

64. The method of any one of claims 1-63, wherein the immunoconjugate is administered subcutaneously or intratumorally.

65. The method of claim 64, wherein the immunoconjugate is administered subcutaneously in combination with hyaluronidase.

66. The method of any one of claims 1-65, wherein the individual is a human.

67. The method of any one of claims 1-66, wherein the individual has cancer, an infectious disease, or a viral associated disease.

68. The method of any one of claims 1-67, wherein the composition of nucleated cells and/or the immunoconjugate is administered prior to, concurrently with, or following administration of another therapy.

69. The method of claim 68, wherein the another therapy is a chemotherapy or a radiation therapy.

70. A composition comprising nucleated cells comprising an exogenous antigen for use in a method for treating a disease in an individual, wherein the composition is used in combination with an immunoconjugate comprising a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19).

71. The composition of claim 70, wherein the disease is cancer, an infectious disease, or a viral-associated disease.

72. The composition of claim 70 or 71, wherein the composition comprising nucleated cells is administered before, at the same time, or after the immunoconjugate.

73. The composition of any one of claims 70-72, wherein the immunoconjugate is administered a plurality of times after administration of the composition comprising nucleated cells.

74. The composition of any one of claims 70-73, wherein the second polypeptide binds a T cell.

75. The composition of any one of claims 70-74, wherein the second polypeptide binds PD-1 expressed on the T cell.

76. The composition of any one of claims 70-75, wherein the second polypeptide is an antigen-binding moiety that specifically binds PD-1.

77. The composition of claim 76, wherein the anti-PD-1 antigen-binding moiety comprises
(a) a heavy chain variable region (VH) comprising a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6; or
(b) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO:13.

78. The composition of claim 76 or 77, wherein the anti-PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO:14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO:17, and SEQ ID NO:18.

79. The composition of any one of claims 76-78, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO:15, SEQ ID NO:16, SEQ ID NO:17, and SEQ ID NO:18.

80. The composition of any one of claims 76-79, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO:15.

81. The composition of any one of claims 70-80, wherein the immunoconjugate comprises a polypeptide comprising an amino acid sequence that is at least about 80%, 85%, 90%, 95%, 96%, 97%, 98%, 99% or 100% identical to the sequence of SEQ ID NO:24, a polypeptide comprising an amino acid sequence that is at least about 80%, 85%, 90%, 95%, 96%, 97%, 98%, 99% or 100% identical to the sequence of SEQ ID NO:24, and a polypeptide comprising an amino acid sequence that is at least about 80%, 85%, 90%, 95%, 96%, 97%, 98%, 99% or 100% identical to the sequence of SEQ ID NO:25.

82. The composition of any one of claims 70-81, wherein the immunoconjugate comprises a polypeptide sequence of SEQ ID NO:22, a polypeptide sequence of SEQ ID NO:24, and a polypeptide sequence of SEQ ID NO:25.

83. The composition of any one of claims 70-73, wherein the second polypeptide specifically binds a target antigen presented on a tumor cell or in a tumor cell environment.

84. The composition of claim 83, wherein the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), Carcinoembryonic Antigen (CEA) and the Melanoma-associated Chondroitin Sulfate Proteoglycan (MCSP).

85. The composition of any one of claims 70-73 and 83-84, wherein the second polypeptide binds FAP.

86. The composition of any one of claims 70-73 and 83-85, wherein the second polypeptide is an antigen-binding moiety that specifically binds FAP.

87. The composition of any one of claims 70-73 and 83-86, wherein the antigen-binding moiety the specifically bind FAP comprising (i) the heavy chain variable region sequence of SEQ ID NO:29 and the light chain variable region sequence of SEQ ID NO: 28; (ii) the heavy chain variable region sequence of SEQ ID NO:31 and the light chain variable region sequence of SEQ ID NO:30; (iii) the heavy chain variable region sequence of SEQ ID NO: 33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36.

88. The composition of any one of claims 70-73 and 83-87, wherein the antigen-binding moiety the specifically bind FAP comprises (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO: 231; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41.

89. The composition of any one of claims 70-73 and 83-88, wherein antigen-binding moiety the specifically bind FAP comprises the heavy chain variable region sequence of SEQ ID NO:33 and the light chain variable region sequence of SEQ ID NO:32.

90. The composition of any one of claims 70-73 and 83-89, wherein the immunoconjugate comprises a polypeptide sequence of SEQ ID NO:38, a polypeptide sequence of SEQ ID NO:39, and a polypeptide sequence of SEQ ID NO:40.

91. The composition of any one of claims 70-90, wherein the mutant IL-2 polypeptide further comprises the amino acid substitution T3A and/or the amino acid substitution C125A.

92. The composition of any one of claims 70-91, wherein the mutant IL-2 polypeptide comprises an amino acid sequence that is at least about 95%, 96%, 97%, 98%, 99% or 100% identical to the amino acid sequence of SEQ ID NO:20, wherein the mutant IL-2 polypeptide shows reduced affinity to the high-affinity IL-2 receptor and substantially similar affinity to the intermediate-affinity IL-2 receptor, each compared to a wild-type IL-2 polypeptide.

93. The composition of any one of claims 70-92, wherein the mutant IL-2 polypeptide comprises the amino acid sequence of SEQ ID NO:20.

94. The composition of any one of claims 70-93, wherein the nucleated cells are immune cells.

95. The composition of any one of claims 70-94, wherein the nucleated cells are a plurality of peripheral blood mononuclear cells (PBMCs).

96. The composition of claim 95, wherein the plurality of PBMCs comprise two or more of T cell, B cell, NK cell, monocytes, dendritic cells or NK-T cells.

97. The composition of any one of claims 70-94, wherein the nucleated cells are one or more of T cells, B cells, NK cells, monocytes, dendritic cells and/or NK-T cells.

98. The composition of any one of claims 70-97, wherein the exogenous antigen is delivered to the nucleated cells intracellularly.

99. The composition of any one of claims 70-98, wherein the exogenous antigen is a disease-associated antigen.

100. The composition of any one of claims 70-99, wherein the disease-associated antigen is a cancer antigen, an infectious disease antigen, or a viral-associated disease antigen.

101. The composition of any one of claims 70-100, wherein the exogenous antigen is a human papillomavirus (HPV) antigen.

102. The composition of any one of claims 70-101, wherein the composition further comprises an adjuvant.

103. The composition of claim 102, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

104. The composition of any one of claims 70-103, wherein the nucleated cells comprising the exogenous antigen are prepared by a process comprising the steps of:
a) passing a cell suspension comprising input nucleated cells through a cell-deforming constriction, wherein a diameter of the constriction is a function of a diameter of the input nucleated cells in the suspension, thereby causing perturbations of the input nucleated cells large enough for the exogenous antigen to pass through to form perturbed input nucleated cells;
b) incubating the perturbed input nucleated cells with the exogenous antigen for a sufficient time to allow the exogenous antigen to enter the perturbed input nucleated cells, thereby generating nucleated cells comprising the exogenous antigen.

105. The composition of claim 104, wherein the width of the constriction is about 10% to about 99% of the mean diameter of the input nucleated cells.

106. The composition of claim 104 or 105, wherein the width of the constriction is about 4.2 μm to about 6 μm.

107. The composition of any one of claims 104-106, wherein the width of the constriction is about 4.2 μm to about 4.8 μm.

108. The composition of any one of claims 104-107, wherein the width of the constriction is about 4.5 μm.

109. The composition of any one of claims 104-108, wherein the cell suspension comprising the plurality of input nucleated cells are passed through multiple constrictions wherein the multiple constrictions are arranged in series and/or in parallel.

110. The composition of any one of claims 104-109, wherein the exogenous antigen is present in at least about 70% of the nucleated cells following incubation of the perturbed input nucleated cells with the exogenous antigen.

111. The composition of any one of claims 70-110, wherein the nucleated cells are conditioned with an adjuvant to form conditioned cells.

112. The composition of claim 111, wherein the nucleated cells are incubated with the adjuvant for about 1 hour to about 24 hours, about 2 hours to about 10 hours, about 3 hours to about 6 hours, or about 4 hours for the cells to condition.

113. The composition of claim 111 or 112, wherein the nucleated cells are conditioned before or after introducing the exogenous antigen into the nucleated cells.

114. The composition of any one of claims 111-113, wherein the adjuvant is a CpG oligodeoxynucleotide (ODN), LPS, IFN-α, STING agonists, RIG-I agonists, poly I.C, R837, R848, a TLR3 agonist, a TLR4 agonist or a TLR 9 agonist.

115. The composition of any one of claims 111-114, wherein the adjuvant is a CpG oligodeoxynucleotide (ODN).

116. The composition of any one of claims 111-115, wherein the adjuvant is CpG 7909.

117. The composition of any one of claims 111-116, wherein the conditioned cells are a conditioned plurality of modified PBMCs.

118. The composition of claim 117, wherein the plurality of modified PBMCs are further modified to increase expression of one or more of co-stimulatory molecules.

119. The composition of claim 118, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

120. The composition of any one of claims 117-119, wherein the plurality of modified PBMCs are further modified to increase expression of one or more cytokines.

121. The composition of claim 120, wherein the cytokine is IL-15, IL-12, IL-2, IFN-α, or IL-21.

122. The composition of any one of claims 117-123, wherein one or more co-stimulatory molecules is upregulated in the B cells of the conditioned plurality of modified PBMCs compared to the B cells in the plurality of nonmodified PBMCs, wherein the co-stimulatory molecule is CD80 and/or CD86.

123. The composition of any one of claims 117-124, wherein the plurality of modified PBMCs have increased expression of one or more of IFN-γ, IL-6, MCP-1, MIP-1β, IP-10, or TNF-α compared to a plurality of unconditioned PBMCs.

124. The composition of any one of claims 117-123, wherein the expression of one or more of IFN-γ, IL-6, MCP-1, MIP-1β, IP-10, or TNF-α is increased by more than about 1.2-fold, 1.5-fold, 1.8-fold, 2-fold, 3-fold, 4-fold, 5-fold, 8-fold, or more than 10-fold compared to the plurality of unconditioned PBMCs.

125. The composition of any one of claims 70-124, wherein the composition and/or the immunoconjugate is administered intravenously.

126. The composition of any one of claims 70-125, wherein the immunoconjugate is administered subcutaneously or intratumorally.

127. The composition of claim 126, wherein the immunoconjugate is administered subcutaneously in combination with hyaluronidase.

128. The composition of any one of claims 70-127, wherein the individual is a human.

129. The composition of any one of claims 70-128, wherein the individual has cancer, an infectious disease, or a viral associated disease.

130. The composition of any one of claims 70-129, wherein the composition is administered prior to, concurrently with, or following administration of another therapy.

131. The composition of claim 130, wherein the another therapy is a chemotherapy or a radiation therapy.

132. An immunoconjugate comprising a mutant IL-2 polypeptide and a second polypeptide capable of specific binding to a T cell, a tumor cell, or the tumor cell environment for use in a method for treating a disease in an individual, wherein the immunoconjugate is used in combination with a composition comprising nucleated cells comprising an exogenous antigen.

133. The immunoconjugate of claim 132, wherein the disease is cancer, an infectious disease, or a viral-associated disease.

134. The immunoconjugate of claim 132 or 133, wherein the composition comprising the nucleated cells is administered before, at the same time, or after the immunoconjugate.

135. The immunoconjugate of any one of claims 132-134, wherein the immunoconjugate is administered a plurality of times after administration of the composition comprising nucleated cells.

136. The immunoconjugate of any one of claims 132-135, wherein the second polypeptide binds a T cell.

137. The immunoconjugate of any one of claims 132-136, wherein the second polypeptide binds PD-1 expressed on the T cell.

138. The immunoconjugate of any one of claims 132-137, wherein the second polypeptide is an antigen-binding moiety that specifically binds PD-1.

139. The immunoconjugate of claim 138, wherein the anti-PD-1 antigen-binding moiety comprises
(a) a heavy chain variable region (VH) comprising a HVR-H1 comprising the amino acid sequence of SEQ ID NO: 1, a HVR-H2 comprising the amino acid sequence of SEQ ID NO:2, a HVR-H3 comprising the amino acid sequence of SEQ ID NO:3, and a FR-H3 comprising the amino acid sequence of SEQ ID NO:7 at positions 71-73 according to Kabat numbering, and a light chain variable region (VL) comprising a HVR-L1 comprising the amino acid sequence of SEQ ID NO:4, a HVR-L2 comprising the amino acid sequence of SEQ ID NO:5, and a HVR-L3 comprising the amino acid sequence of SEQ ID NO:6; or
(b) a heavy chain variable region (VH) comprising a HVR-H1 comprising the amino acid sequence of SEQ ID NO:8, a HVR-H2 comprising the amino acid sequence of SEQ ID NO:9, and a HVR-H3 comprising the amino acid sequence of SEQ ID NO: 10, and a light chain variable region (VL) comprising a HVR-L1 comprising the amino acid sequence of SEQ ID NO: 11, a HVR-L2 comprising the amino acid sequence of SEQ ID NO: 12, and a HVR-L3 comprising the amino acid sequence of SEQ ID NO:13.

140. The immunoconjugate of claim 138 or 139, wherein the anti-PD-1 antigen-binding moiety comprises (a) a heavy chain variable region (VH) comprising an amino acid sequence that is at least about 95%, 96%, 97%, 98%, 99% or 100% identical to the amino acid sequence of SEQ ID NO:14, and (b) a light chain variable region (VL) comprising an amino acid sequence that is at least about 95%, 96%, 97%, 98%, 99% or 100% identical to an amino acid sequence selected from the group consisting of SEQ ID NO:15, SEQ ID NO:16, SEQ ID NO:17, and SEQ ID NO:18.

141. The immunoconjugate of any one of claims 138-140, wherein the anti-PD-1 antigen-binding moiety comprises (a) a heavy chain variable region (VH) comprising an amino acid sequence of SEQ ID NO:14, and (b) a light chain variable region (VL) comprising an amino acid sequence of SEQ ID NO:15, SEQ ID NO:16, SEQ ID NO:17, and SEQ ID NO: 18.

142. The immunoconjugate of any one of claims 138-141, wherein the anti-PD-1 antigen-binding moiety comprises (a) a heavy chain variable region (VH) comprising an amino acid sequence of SEQ ID NO: 14, and (b) a light chain variable region (VL) comprising an amino acid sequence of SEQ ID NO:15.

143. The immunoconjugate of any one of claims 132-142, wherein the immunoconjugate comprises a polypeptide comprising an amino acid sequence that is at least about 80%, 85%, 90%, 95%, 96%, 97%, 98%, 99% or 100% identical to the sequence of SEQ ID NO:22, a polypeptide comprising an amino acid sequence that is at least about 80%, 85%, 90%, 95%, 96%, 97%, 98%, 99% or 100% identical to the sequence of SEQ ID NO:24, and a polypeptide comprising an amino acid sequence that is at least about 80%, 85%, 90%, 95%, 96%, 97%, 98%, 99% or 100% identical to the sequence of SEQ ID NO:25.

144. The immunoconjugate of any one of claims 132-143, wherein the immunoconjugate comprises a polypeptide sequence of SEQ ID NO:22, a polypeptide sequence of SEQ ID NO:24, and a polypeptide sequence of SEQ ID NO:25.

145. The immunoconjugate of any one of claims 132-135, wherein the second polypeptide specifically binds a target antigen presented on a tumor cell or in the tumor cell environment.

146. The immunoconjugate of any one of claims 132-135 and 145, wherein the target antigen is selected from the group consisting of a Fibroblast activation protein (FAP), the A1 domain of Tenascin-C (TNC A1), the A2 domain of Tenascin-C (TNC A2), the Extra Domain B of Fibronectin (EDB), the Carcinoembryonic Antigen (CEA) and the Melanoma-associated Chondroitin Sulfate Proteoglycan (MCSP).

147. The immunoconjugate of any one of claims 132-135 and 145-146, wherein the second polypeptide binds FAP.

148. The immunoconjugate of any one of claims 132-135 and 145-147, wherein the second polypeptide is an antigen-binding moiety that specifically binds FAP.

149. The immunoconjugate of claim 148, wherein the antigen-binding moiety the specifically bind FAP comprising (i) the heavy chain variable region sequence of SEQ ID NO:29 and the light chain variable region sequence of SEQ ID NO: 28; (ii) the heavy chain variable region sequence of SEQ ID NO:31 and the light chain variable region sequence of SEQ ID NO:30; (iii) the heavy chain variable region sequence of SEQ ID NO: 33 and the light chain variable region sequence of SEQ ID NO:32; (iv) the heavy chain variable region sequence of SEQ ID NO:35 and the light chain variable region sequence of SEQ ID NO:34; or (v) the heavy chain variable region sequence of SEQ ID NO:37 and the light chain variable region sequence of SEQ ID NO:36.

150. The immunoconjugate of claim 148 or 149, wherein the antigen-binding moiety the specifically bind FAP comprising (i) a polypeptide sequence that is at least 80%, 85%, 90%, 95%, 96%, 97%, 98%, 99% or 100% identical to SEQ ID NO:42, a polypeptide sequence that is at least 80%, 85%, 90%, 95%, 96%, 97%, 98%, 99% or 100% identical to SEQ ID NO:43, and a polypeptide sequence that is at least 80%, 85%, 90%, 95%, 96%, 97%, 98%, 99% or 100% identical to SEQ ID NO:41; (ii) a polypeptide sequence that is at least 80%, 85%, 90%, 95%, 96%, 97%, 98%, 99% or 100% identical to SEQ ID NO:38, a polypeptide sequence that is at least 80%, 85%, 90%, 95%, 96%, 97%, 98%, 99% or 100% identical to SEQ ID NO:39, and a polypeptide sequence that is at least 80%, 85%, 90%, 95%, 96%, 97%, 98%, 99% or 100% identical to SEQ ID NO: 231; or (iii) a polypeptide sequence that is at least 80%, 85%, 90%, 95%, 96%, 97%, 98%, 99% or 100% identical to SEQ ID NO:44, a polypeptide sequence that is at least 80%, 85%, 90%, 95%, 96%, 97%, 98%, 99% or 100% identical to SEQ ID NO:45, and a polypeptide sequence that is at least 80%, 85%, 90%, 95%, 96%, 97%, 98%, 99% or 100% identical to SEQ ID NO:41.

151. The immunoconjugate of any one of claims 148-150, wherein antigen-binding moiety the specifically bind FAP comprises the heavy chain variable region sequence of SEQ ID NO:33 and the light chain variable region sequence of SEQ ID NO:32.

152. The immunoconjugate of any one of claims 132-135 and 145-151, wherein the immunoconjugate comprises a polypeptide sequence of SEQ ID NO:38, a polypeptide sequence of SEQ ID NO:39, and a polypeptide sequence of SEQ ID NO:40.

153. The immunoconjugate of any one of claims 132-152, wherein the mutant IL-2 polypeptide further comprises the amino acid substitution T3A and/or the amino acid substitution C125A.

154. The immunoconjugate of any one of claims 132-153, wherein the mutant IL-2 polypeptide comprises an amino acid sequence that is at least about 95%, 96%, 97%, 98%, 99% or 100% identical to the amino acid sequence of SEQ ID NO:20, wherein the mutant IL-2 polypeptide shows reduced affinity to the high-affinity IL-2 receptor and substantially similar affinity to the intermediate-affinity IL-2 receptor, each compared to a wild-type IL-2 polypeptide.

155. The immunoconjugate of any one of claims 132-154, wherein the mutant IL-2 polypeptide comprises the amino acid sequence of SEQ ID NO:20.

156. The immunoconjugate of any one of claims 132-155, wherein the nucleated cells are immune cells.

157. The immunoconjugate of any one of claims 132-156, wherein the nucleated cells are a plurality of peripheral blood mononuclear cells (PBMCs).

158. The immunoconjugate of claim 157, wherein the plurality of PBMCs comprise two or more of T cell, B cell, NK cell, monocytes, dendritic cells or NK-T cells.

159. The immunoconjugate of any one of claims 132-156, wherein the nucleated cells are one or more of T cells, B cells, NK cells, monocytes, dendritic cells and/or NK-T cells.

160. The immunoconjugate of any one of claims 132-159, wherein the exogenous antigen is delivered to the nucleated cells intracellularly.

161. The immunoconjugate of any one of claims 132-160, wherein the exogenous antigen is a disease-associated antigen.

162. The immunoconjugate of any one of claims 132-161, wherein the disease-associated antigen is a cancer antigen, an infectious disease antigen, or a viral-associated disease antigen.

163. The immunoconjugate of any one of claims 132-162, wherein the exogenous antigen is a human papillomavirus (HPV) antigen.

164. The immunoconjugate of any one of claims 132-163, wherein the composition further comprises an adjuvant.

165. The immunoconjugate of claim 164, wherein the adjuvant is a CpG oligodeoxynucleotide (ODN), LPS, IFN-α, STING agonists, RIG-I agonists, poly I:C, R837, R848, a TLR3 agonist, a TLR4 agonist or a TLR 9 agonist.

166. The immunoconjugate of any one of claims 132-165, wherein the nucleated cells comprising the exogenous antigen are prepared by a process comprising the steps of:
a) passing a cell suspension comprising input nucleated cells through a cell-deforming constriction, wherein a diameter of the constriction is a function of a diameter of the input nucleated cells in the suspension, thereby causing perturbations of the input nucleated cells large enough for the exogenous antigen to pass through to form perturbed input nucleated cells;
b) incubating the perturbed input nucleated cells with the exogenous antigen for a sufficient time to allow the exogenous antigen to enter the perturbed input nucleated cells, thereby generating nucleated cells comprising the exogenous antigen.

167. The immunoconjugate of claim 166, wherein the width of the constriction is about 10% to about 99% of the mean diameter of the input nucleated cells.

168. The immunoconjugate of claim 166 or 167, wherein the width of the constriction is about 4.2 μm to about 6 μm.

169. The immunoconjugate of any one of claims 166-168, wherein the width of the constriction is about 4.2 μm to about 4.8 μm.

170. The immunoconjugate of any one of claims 166-169, wherein the width of the constriction is about 4.5 μm.

171. The immunoconjugate of any one of claims 166-170, wherein the cell suspension comprising the plurality of input nucleated cells are passed through multiple constrictions wherein the multiple constrictions are arranged in series and/or in parallel.

172. The immunoconjugate of any one of claims 166-171, wherein the exogenous antigen is present in at least about 70% of the nucleated cells following incubation of the perturbed input nucleated cells with the exogenous antigen.

173. The immunoconjugate of any one of claims 132-172, wherein the nucleated cells are conditioned with an adjuvant to form conditioned cells.

174. The immunoconjugate of claim 173, wherein the nucleated cells are incubated with the adjuvant for about 1 hour to about 24 hours, about 2 hours to about 10 hours, about 3 hours to about 6 hours, or about 4 hours for the cells to condition.

175. The immunoconjugate of claim 173 or 174, wherein the nucleated cells are conditioned before or after introducing the exogenous antigen into the nucleated cells.

176. The immunoconjugate of any one of claims 173-175, wherein the adjuvant is a CpG oligodeoxynucleotide (ODN), LPS, IFN-α, STING agonists, RIG-I agonists, poly I.C, R837, R848, a TLR3 agonist, a TLR4 agonist or a TLR 9 agonist.

177. The immunoconjugate of any one of claims 173-176, wherein the adjuvant is a CpG oligodeoxynucleotide (ODN).

178. The immunoconjugate of any one of claims 173-177, wherein the adjuvant is CpG 7909.

179. The immunoconjugate of any one of claims 173-178, wherein the conditioned cells are a conditioned plurality of modified PBMCs.

180. The immunoconjugate of claim 179, wherein the plurality of modified PBMCs are further modified to increase expression of one or more of co-stimulatory molecules.

181. The immunoconjugate of claim 180, wherein the co-stimulatory molecule is B7-H2 (ICOSL), B7-1 (CD80), B7-2 (CD86), CD70, LIGHT, HVEM, CD40, 4-1BBL, OX40L, TL1A, GITRL, CD30L, TIM4, SLAM, CD48, CD58, CD155, or CD112.

182. The immunoconjugate of any one of claims 179-181, wherein the plurality of modified PBMCs are further modified to increase expression of one or more cytokines.

183. The immunoconjugate of claim 182, wherein the cytokine is IL-15, IL-12, IL-2, IFN-α, or IL-21.

184. The immunoconjugate of any one of claims 179-183, wherein one or more co-stimulatory molecules is upregulated in the B cells of the conditioned plurality of modified PBMCs compared to the B cells in the plurality of nonmodified PBMCs, wherein the co-stimulatory molecule is CD80 and/or CD86.

185. The immunoconjugate of any one of claims 179-184, wherein the plurality of modified PBMCs have increased expression of one or more of IFN-γ, IL-6, MCP-1, MIP-1β, IP-10, or TNF-α compared to a plurality of unconditioned PBMCs.

186. The immunoconjugate of any one of claims 179-185, wherein the expression of one or more of IFN-γ, IL-6, MCP-1, MIP-1β, IP-10, or TNF-α is increased by more than about 1.2-fold, 1.5-fold, 1.8-fold, 2-fold, 3-fold, 4-fold, 5-fold, 8-fold, or more than 10-fold compared to the plurality of unconditioned PBMCs.

187. The immunoconjugate of any one of claims 132-186, wherein the composition and/or the immunoconjugate is administered intravenously.

188. The immunoconjugate of any one of claims 132-186, wherein the immunoconjugate is administered subcutaneously or intratumorally.

189. The immunoconjugate of claim 188, wherein the immunoconjugate is administered subcutaneously in combination with hyaluronidase.

190. The immunoconjugate of any one of claims 132-189, wherein the individual is a human.

191. The immunoconjugate of any one of claims 132-190, wherein the individual has cancer, an infectious disease, or a viral associated disease.

192. The immunoconjugate of any one of claims 132-191, wherein the composition is administered prior to, concurrently with, or following administration of another therapy.

193. The immunoconjugate of claim 192, wherein the another therapy is a chemotherapy or a radiation therapy.

194. Use of an effective amount of an immunoconjugate in the manufacture of a medicament for stimulating an immune response in an individual, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19);
wherein the immunoconjugate is formulated for administration in conjunction with a composition comprising nucleated cells, wherein the nucleated cells comprise an exogenous antigen.

195. The use of claim 194, wherein the immunoconjugate is administered before, at the same time, or after the composition comprising nucleated cells.

196. Use of an effective amount of a composition in the manufacture of a medicament for stimulating an immune response in an individual, wherein the composition comprises nucleated cells comprising an exogenous antigen;
wherein the composition is formulated for administration in conjunction with an immunoconjugate comprising a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19).

197. The composition of claim 196, wherein the composition comprising the nucleated cells is administered before, at the same time, or after the immunoconjugate.

198. A kit for use in the method of any one of claims 1-69.

199. A kit comprising a composition of nucleated cells comprising an exogenous antigen and an immunoconjugate, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19); wherein the composition and the immunoconjugate are for use in combination for stimulating an immune response to the exogenous antigen in an individual.

200. A kit comprising a composition of nucleated cells comprising an exogenous antigen, wherein the composition is for use in conjunction with an immunoconjugate for stimulating an immune response to the exogenous antigen in an individual;
wherein the immunoconjugate comprises a mutant IL-2 polypeptide and a second polypeptide capable of specific binding to a T cell, a tumor cell, or the tumor cell environment; and wherein the mutant IL-2 polypeptide is a human IL-2 molecule comprising the amino acid substitutions F42A, Y45A and L72G (numbering relative to the human IL-2 sequence SEQ ID NO: 19).

201. A kit comprising an immunoconjugate, wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19);
wherein the immunoconjugate is for use in conjunction with a composition of nucleated cells comprising an exogenous antigen for stimulating an immune response to the exogenous antigen in an individual.

202. A method for producing an immunoconjugate for use in conjunction with a composition comprising nucleated cells for stimulating an immune response in an individual, the method comprising expressing a nucleic acid encoding the immunoconjugate in a cell under conditions to produce the immunoconjugate,
wherein the immunoconjugate comprises a mutant IL-2 polypeptide and a second polypeptide capable of specific binding to a T cell, a tumor cell, or the tumor cell environment; wherein the mutant IL-2 polypeptide is a human IL-2 molecule comprising the amino acid substitutions F42A, Y45A and L72G (numbering relative to the human IL-2 sequence SEQ ID NO: 19);
wherein the immunoconjugate is for use in conjunction with administering a composition comprising nucleated cells, wherein the nucleated cells comprise an exogenous antigen.

203. The method of claim 202, wherein the immunoconjugate is a fusion protein.

204. A method for producing a composition comprising nucleated cells for use in conjunction with an immunoconjugate for stimulating an immune response in an individual, the method comprising introducing an exogenous antigen intracellularly to a population of nucleated cells;
wherein the composition is for use in conjunction with administration of an immunoconjugate;
wherein the immunoconjugate comprises a mutant IL-2 polypeptide and a second polypeptide capable of specific binding to a T cell, a tumor cell, or the tumor cell environment; and wherein the mutant IL-2 polypeptide is a human IL-2 molecule comprising the amino acid substitutions F42A, Y45A and L72G (numbering relative to the human IL-2 sequence SEQ ID NO: 19).


SEQUENCE LISTING (73 sequences)

SEQ ID NO: 1 - protein, 4 aa, Artificial Sequence
  Synthetic Construct
SSYT

SEQ ID NO: 2 - protein, 8 aa, Artificial Sequence
  Synthetic Construct
SGGGRDIY

SEQ ID NO: 3 - protein, 5 aa, Artificial Sequence
  Synthetic Construct
GRVYF

SEQ ID NO: 4 - protein, 6 aa, Artificial Sequence
  Synthetic Construct
TSDNSF

SEQ ID NO: 5 - protein, 7 aa, Artificial Sequence
  Synthetic Construct
RSSTLES

SEQ ID NO: 6 - protein, 6 aa, Artificial Sequence
  Synthetic Construct
NYDVPW

SEQ ID NO: 7 - protein, 3 aa, Artificial Sequence
  Synthetic Construct
RDN

SEQ ID NO: 8 - protein, 7 aa, Artificial Sequence
  Synthetic Construct
GFSFSSY

SEQ ID NO: 9 - protein, 3 aa, Artificial Sequence
  Synthetic Construct
GGR

SEQ ID NO: 10 - protein, 9 aa, Artificial Sequence
  Synthetic Construct
TGRVYFALD

SEQ ID NO: 11 - protein, 11 aa, Artificial Sequence
  Synthetic Construct
SESVDTSDNSF

SEQ ID NO: 12 - protein, 3 aa, Artificial Sequence
  Synthetic Construct
RSS

SEQ ID NO: 13 - protein, 6 aa, Artificial Sequence
  Synthetic Construct
NYDVPW

SEQ ID NO: 14 - protein, 120 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 15 - protein, 111 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 16 - protein, 111 aa, Artificial Sequence
  Synthetic Construct
DVVMTQSPLSLPVTLGQPASISCRASESVDTSDNSFIHWYQQRPGQSPRLLIYRSSTLESGVPDRFSGSGSGTDFTLKISRVEAEDVGVYYCQQNYDVPWTFGQGTKVEIK

SEQ ID NO: 17 - protein, 111 aa, Artificial Sequence
  Synthetic Construct
EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIK

SEQ ID NO: 18 - protein, 111 aa, Artificial Sequence; identical to SEQ ID NO: 17
  Synthetic Construct

SEQ ID NO: 19 - protein, 133 aa, Homo sapiens [sequence omitted]

SEQ ID NO: 20 - protein, 133 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 21 - protein, 15 aa, Artificial Sequence
  Synthetic Construct
GGGGSGGGGSGGGGS

SEQ ID NO: 22 - protein, 597 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 23 - protein, 448 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 24 - protein, 448 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSP

SEQ ID NO: 25 - protein, 218 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 266 aa, Artificial Sequence
  Synthetic Construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 27 - protein, 133 aa, Artificial Sequence; identical to SEQ ID NO: 20
  Synthetic Construct

SEQ ID NO: 28 - protein, 108 aa, Artificial Sequence
  Synthetic Construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 29 - protein, 117 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSS

SEQ ID NO: 30 - protein, 108 aa, Artificial Sequence
  Synthetic Construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 31 - protein, 117 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSS

SEQ ID NO: 32 - protein, 108 aa, Artificial Sequence; identical to SEQ ID NO: 28
  Synthetic Construct

SEQ ID NO: 33 - protein, 117 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 34 - protein, 108 aa, Artificial Sequence; identical to SEQ ID NO: 30
  Synthetic Construct

SEQ ID NO: 35 - protein, 116 aa, Artificial Sequence
  Synthetic Construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 36 - protein, 117 aa, Artificial Sequence
  Synthetic Construct
EVQLLESGGGLVQPGGSLRLSCAASGFTFSSYAMSWVRQAPGKGLEWVSAIIGSGGITYYADSVKGRFTISRDNSKNTLYLQMNSLRAEDTAVYYCAKGWFGGFNYWGQGTLVTVSS

SEQ ID NO: 37 - protein, 117 aa, Artificial Sequence; identical to SEQ ID NO: 36
  Synthetic Construct

SEQ ID NO: 38 - protein, 594 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 39 - protein, 447 aa, Artificial Sequence
  Synthetic Construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 214 aa, Artificial Sequence
  Synthetic Construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTTLSKADYEKHKVYACEVTHQGLSSPVTKSFNRGEC

SEQ ID NO: 41 - protein, 215 aa, Artificial Sequence
  Synthetic Construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 593 aa, Artificial Sequence
  Synthetic Construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 43 - protein, 446 aa, Artificial Sequence
  Synthetic Construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 594 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 45 - protein, 447 aa, Artificial Sequence
  Synthetic Construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWLGNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 10 aa, Homo sapiens
TIHDIILECV

SEQ ID NO: 47 - protein, 10 aa, Mus musculus
EVYDFAFRDL

SEQ ID NO: 48 - protein, 10 aa, Homo sapiens
YMLDLQPETT

SEQ ID NO: 49 - protein, 9 aa, Mus musculus
RAHYNIVTF

SEQ ID NO: 50 - protein, 36 aa, Homo sapiens
LPQLSTELQTTIHDIILECVYSKQQLLRREVYDFAF

SEQ ID NO: 51 - protein, 25 aa, Homo sapiens
QLCTELQTTIHDIILECVYCKQQLL

SEQ ID NO: 52 - protein, 25 aa, Mus musculus
KQQLLRREVYDFAFRDLCIVYRDGN

SEQ ID NO: 53 - protein, 35 aa, Mus musculus
VYSKQQLLRREVYDFAFRDLSIVYRDGNPYAVSDK

SEQ ID NO: 54 - protein, 35 aa, Homo sapiens
MHGDTPTLHEYMLDLQPETTDLYCYEQLNDSSEEE

SEQ ID NO: 55 - protein, 25 aa, Homo sapiens
QLCTELQTYMLDLQPETTYCKQQLL

SEQ ID NO: 56 - protein, 35 aa, Mus musculus
GQAEPDRAHYNIVTFCCKCDSTLRLCVQSTHVDIR

SEQ ID NO: 57 - protein, 35 aa, Mus musculus
GQAEPDRAHYNIVTFSSKSDSTLRLSVQSTHVDIR

SEQ ID NO: 58 - DNA, 20 bp, Artificial Sequence
  Synthetic Construct
tccatgagcttcctgacgtt

SEQ ID NO: 59 - protein, 20 aa, Artificial Sequence
  Synthetic Construct
MYRMQLLSCIALSLALVTNS

SEQ ID NO: 60 - protein, 10 aa, Homo sapiens
LPQLSTELQT

SEQ ID NO: 61 - protein, 8 aa, Homo sapiens
QLCTELQT

SEQ ID NO: 62 - protein, 7 aa, Mus Musculus
KQQLLRR

SEQ ID NO: 63 - protein, 10 aa, Mus Musculus
VYSKQQLLRR

SEQ ID NO: 64 - protein, 10 aa, Homo sapiens
MHGDTPTLHE

SEQ ID NO: 65 - protein, 6 aa, Mus Musculus
GQAEPD

SEQ ID NO: 66 - protein, 16 aa, Homo sapiens
YSKQQLLRREVYDFAF

SEQ ID NO: 67 - protein, 7 aa, Homo sapiens
YCKQQLL

SEQ ID NO: 68 - protein, 8 aa, Mus musculus
CIVYRDGN

SEQ ID NO: 69 - protein, 15 aa, Mus musculus
SIVYRDGNPYAVSDK

SEQ ID NO: 70 - protein, 15 aa, Homo sapiens
DLYCYEQLNDSSEEE

SEQ ID NO: 71 - protein, 20 aa, Mus musculus
CCKCDSTLRLCVQSTHVDIR

SEQ ID NO: 72 - protein, 20 aa, Mus musculus
SSKSDSTLRLSVQSTHVDIR

SEQ ID NO: 73 - DNA, 24 bp, Artificial Sequence
  Synthetic Construct
tcgtcgttttgtcgttttgtcgtt